(19) United States
(12) Patent Application Publication
(10) Pub. No.: US 2022/0378913 A1
(43) Pub. Date: Dec. 1, 2022

(54) METHODS FOR DIAGNOSIS AND TREATMENT

(71) Applicant: GMDx Co Pty Ltd, Melbourne, Victoria (AU)

(72) Inventors: Robyn LINDLEY, Melbourne, Victoria (AU); Nathan HALL, Melbourne, Victoria (AU); Jared MAMROT, Melbourne, Victoria (AU)

(21) Appl. No.: 17/771,680

(22) PCT Filed: Oct. 26, 2020
(86) PCT No.: PCT/AU2020/051149

§ 371 Date: Apr. 25, 2022

(30) Foreign Application Priority Data
Oct. 25, 2019 (AU) 2019904028

(51) Int. Cl. A61K 41/00; C12Q 1/6883; A61P 25/16; A61P 25/28

(52) U.S. Cl. CPC A61K 41/00; C12Q 1/6883; A61P 25/16; A61P 25/28

(57) ABSTRACT

Systems and methods for diagnosing and treating a neurodegenerative disorder in a subject can be used for the diagnosis of Mild Cognitive Impairment, Early Mild Cognitive Impairment, Late Mild Cognitive Impairment, Parkinson's Disease, Dementia or Alzheimer's Disease in a subject, and for the treatment of a subject diagnosed with such neurodegenerative diseases.

### RELATED APPLICATIONS

This application claims priority to Australian Provisional Application No. 2019904028 entitled “Methods for diagnosis and treatment” filed 25 Oct. 2019, the content of which is incorporated herein by reference in its entirety.

### FIELD OF THE INVENTION

This invention relates generally to systems and methods for diagnosing a neurodegenerative disorder in a subject. In particular embodiments, the methods of the disclosure can be used to for the diagnosis of Mild Cognitive Impairment (MCI), Early Mild Cognitive Impairment (EMCI), Late Mild Cognitive Impairment (LMCI), Parkinson's Disease (PD), Dementia or Alzheimer's Disease. In other embodiments, the methods involve treatment of a subject diagnosed with such diseases.

### BACKGROUND OF THE INVENTION

Neurodegenerative disorders cause significant morbity and mortality throughout the world. Worldwide, more than 44 million people are estimated to be living with Alzheimer's disease (AD) and related disorders—the most common class of neurodegenerative diseases—and this figure is expected to significantly increase in the coming decades. Indeed, it is estimated that only 25% of people with AD have been diagnosed, and the number of people with AD and dementia is expected to almost double over the next 20 years. AD and other dementias are the top cause for disabilities in later life and are the cause of more deaths than breast and prostate cancers combined. Moreover, people with AD are hospitalized three times more often than seniors without the disease.

Neurodegenerative diseases such as AD and Parkinson's disease (PD) are a global health, economic and social emergency with an unmet medical need. There is a need for methods for identifying subjects who have or are likely to develop these and other neurodegenerative diseases so as to facilitate early intervention and management.

### SUMMARY OF THE INVENTION

The present disclosure is predicated on the determination that the number, percentage or ratio of particular types of single nucleotide variants (SNVs) in the nucleic acid of a subject with a neurodegenerative disease or a subject likely to develop a neurodegenerative disease is different to that of a subject who does not have the neurodegenerative disease or a subject that is unlikely to develop a neurodegenerative disease. The SNVs include those that might be attributed to the activity of one or more endogenous deaminases, as well as those that may not necessarily be attributed to the activity of one or more endogenous deaminases.

As described herein, SNVs identified in a nucleic acid molecule can be used to determine a plurality of metrics, which can then in turn be used to help distinguish subjects that have or are likely to develop a neurodegenerative disease. Thus, a profile can be built based upon this plurality of metrics, whereupon subjects that have or are likely to develop a neurodegenerative disease typically have a different profile to subjects that do not have or are unlikely to have a neurodegenerative disease.

In one aspect, provided is a method for determining the likelihood that a subject has or will develop a neurodegenerative disease, comprising: analyzing the sequence of a nucleic acid molecule from a subject to detect SNVs within the nucleic acid molecule; determining a plurality of metrics based on the number and/or type of SNVs detected so as to obtain a subject profile of metrics; and, determining the likelihood of a subject having or developing a neurodegenerative disease on a comparison between the subject profile and a reference profile of metrics;

wherein: the neurodegenerative disease is mild cognitive impairment (MCI) or Alzheimer's disease (AD) and the plurality of metrics comprises those set forth in Table 1 or at least 90% of the metrics set forth in Table 1;

the neurodegenerative disease is early mild cognitive impairment (EMCI) and the plurality of metrics comprises those set forth in Table 2 or at least 90% of the metrics set forth in Table 2;

the neurodegenerative disease is AD and the plurality of metrics comprises those set forth in Table 3 or at least 90% of the metrics set forth in Table 3; or

the neurodegenerative disease is Parkinson's disease (PD) and the plurality of metrics comprises those set forth in any one of Tables 4-6 or at least 90% of the metrics set forth in any one of Tables 4-6.

In some examples, the reference profile is representative of a subject that has or will develop the neurodegenerative disease.

In particular embodiments, the comparison includes assigning a score to each metric that is outside a predetermined range interval, or above or below a predetermined cut-off, for the metric; combining each score to calculate a total score; and comparing the total score to a threshold score, wherein the subject is determined to be likely to have or to develop the neurodegenerative disease when the total score is equal to or more than, or is more than, the threshold score.

In some embodiments, the sequence is a whole genome or whole exome sequence.

In one example, the nucleic acid molecule was obtained from blood, or saliva.

In a further aspect, provided is a method for treating a neurodegerative disease in a subject, the method comprising: (i) performing the method according to any one of claims 1-5; (ii) determining that the subject is likely to have a neurodegenerative disease selected from among MCI, EMCI, Alzheimer's disease and Parkinson's disease; and (iii) exposing the subject to a therapy.

In some examples, the disease is MCI, EMCI or Alzheimer's disease and therapy comprises administration of a cognitive enhancer, an anti-inflammatory, an anti-neuropsychiatric, a cholinesterase inhibitor, an N-methyl-D-aspartate receptor antagonist, an anti-beta amyloid agent (Aβ) agent, and/or an anti-tau agent. In a particular embodiment, the therapy comprises administration of one or more of donepezil, galantamine, rivastigmine, memantine, Aducanumab, levetiracetam, ALZT-OP1, cromolyn+ibuprofen, blarcamesine, AVP-786, AXS-05, Azeliragon, BAN2401, troriluzole, BPDO-1603, Brexpiprazole, CAD106b, COR388, Escitalopram, Gantenerumab, Gantenerumab and solanezumab, Ginkgo biloba, Guanfacine, Icosapent ethyl (IPE), Losartan+amlodipine+atorvastatin, Masitinib, Metformin, Methylphenidate, Mirtazapine, Octohydro-aminoacridine Succinate, Solanezumab, Tricaprilin, TRx0237, or Zolpidem+zoplicone.

In other examples, the disease is Parkinson's disease and therapy comprises administration of levodopa, a dopamine agonist (e.g. bromocriptine, cabergoline, apomorphine, pramipexole, ropinirole, or rotigotine), a monoamine oxidase-B (MAO B) inhibitor (e.g. selegiline, rasagiline or safinamide), a catechol O-methyltransferase (COMT) inhibitor (e.g. entacapone or tolcapone), an anticholinergic (e.g. enztropine or trihexyphenidyl), amantadine, an adenosine A2A antagonist (e.g. istradefylline), Cu-ATSM, a cell therapy (e.g. mesenchymal stem cells, or neural stem cells), a kinase inhibitor (e.g. DNL 151, FB-101, saracatinib), a neurotropic factor (e.g. GDNF or CDNF), or a GLP-1 agonist (e.g. exenatide).

### BRIEF DESCRIPTION OF THE FIGURES

Various examples and embodiments of the present invention will now be described with reference to the accompanying drawings, in which:

FIG. 1 is a graphical representation of the cognitive impairment score given to normal control subjects (CN) or subjects with Alzheimer's disease (AD), dementia, early mild cognitive impairment (EMCI), mild cognitive impairment (MCI), or late mild cognitive impairment (LMCI) on the basis of the metrics shown in Table 1. (A) CI scores for each subject in the cohort. (B) CI Score for each group.

FIG. 2 provides analysis of the differentiation of CN and EMCI subjects on the basis of the metrics shown in Table 2. An EMCI score was given to each subject on the basis of analysis of the metrics in Table 2. (A) Box plot of EMCI scores, compared to control patient scores. (B) Relative proportions (as %) of subjects from each cohort that fall below 23.5, within the range 23.5-26.5, or above 26.5, where each bar in each group represents, from left to right, CN, EMCI, MCI, LMCI, Dementia, and AD.

FIG. 3 provides analysis of the differentiation of CN and AD subjects on the basis of the metrics shown in Table 3. An AD score was given to each subject on the basis of analysis of the metrics in Table 3. (A) Box plot of AD scores. (B) Relative proportions (as %) of subjects from each cohort that fall below 18.5, within the range 18.5-22.5, or above 22.5.

FIG. 4 provides analysis of the differentiation of CN and PD subjects on the basis of the metrics shown in Table 4. A PD score was given to each subject on the basis of analysis of the metrics in Table 4. (A) Box plot of PD scores. (B) Sensitivity and specificity using various PD threshold (or cut-off) scores (ROC curve).

FIG. 5 provides analysis of the differentiation of CN and PD subjects on the basis of the metrics shown in Table 5. A PD score was given to each subject on the basis of analysis of the metrics in Table 5. (A) Box plot of PD scores. (B) Sensitivity and specificity using various PD threshold (or cut-off) scores (ROC curve).

FIG. 6 provides analysis of the differentiation of CN and PD subjects on the basis of the metrics shown in Table 6. A PD score was given to each subject on the basis of analysis of the metrics in Table 6. (A) Box plot of PD scores. (B) Sensitivity and specificity using various PD threshold (or cut-off) scores (ROC curve).

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “a telomere” means one telomere or more than one telomere.

As used herein, “and/or” refers to and encompasses any and all possible combinations of one or more of the associated listed items, as well as the lack of combinations when interpreted in the alternative (or).

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The term “biological sample” as used herein refers to a sample that may be extracted, untreated, treated, diluted or concentrated from a subject or patient. Suitably, the biological sample is selected from any part of a patient's body, including, but not limited to bodily fluids such as saliva or blood, tissue, cells, hair, skin and nails.

As used herein, the term “codon context” with reference to an SNV refers to the nucleotide position within a codon at which the SNV occurs. For the purposes of the present disclosure, the nucleotide positions within an affected codon (MC; i.e., a codon containing the SNV) are annotated MC-1, MC-2 and MC-3, and refer to the first, second and third nucleotide positions, respectively, when the sequence of the codon is read 5′ to 3′. Accordingly, the phrase “determining the codon context of an SNV” or similar phrase means determining at which nucleotide position within the affected codon the SNV occurs, i.e., MC-1, MC-2 or MC-3.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “control subject” or “healthy subject”, as used in the context of the present disclosure refers to a subject known to not have, or to not be at risk of developing, a particular neurodegenerative disease, such as AD, PD, MCI, EMCI, LMCI, or dementia. It is understood that control subjects can be used to obtain data for use as a standard for multiple studies, i.e., it can be used over and over again for multiple different subjects. In other words, for example, when comparing a subject sample to a control sample, the data from the control sample could have been obtained in a different set of experiments, for example, it could be an average obtained from a number of subjects and not actually obtained at the time the data for the test subject was obtained.

The term “correlating” generally refers to determining a relationship between one type of data with another or with a state. In various embodiments, correlating deaminase activity or a profile with the likelihood that a subject has or will develop a neurodegenerative disorder comprises assessing metrics as described herein in a subject and comparing the levels of these metrics to metrics in persons known to be unlikely to have or to develop a neurodegenerative disorder.

By “gene” is meant a unit of inheritance that occupies a specific locus on a genome and comprises transcriptional and/or translational regulatory sequences and/or a coding region and/or non-translated sequences (i.e., introns, 5′ and 3′ untranslated sequences).

As used herein, the term “likelihood” or grammatical variations is used as a measure of whether the subject has or will develop a neurodegenerative disease. An increased likelihood for example may be relative or absolute and may be expressed qualitatively or quantitatively. For instance, an increased likelihood that a subject has or will develop a neurodegenerative disease may be expressed as determining whether the subject has a profile of metric that is essentially the same as or is different to a reference profile, and placing the test subject in an “increased likelihood” category or “decreased likelihood” category.

In some embodiments, the methods comprise comparing a score based on the number of metrics that are outside a predetermined range interval or above or below a cut-off to a “threshold score”. The threshold score is one that provides an acceptable ability to identify a subject as having or developing a neurodegenerative disease, and can be determined by those skilled in the art using any acceptable means. In some examples, receiver operating characteristic (ROC) curves are calculated by plotting the value of a variable versus its relative frequency in two populations in which a first population has a first phenotype or risk and a second population has a second phenotype or risk.

A distribution of the number of metrics that are outside a predetermined range interval or are above or below a cutoff in subjects have or will develop a neurodegenerative disease and in subjects who do not have or will not develop a neurodegenerative disease may overlap. Under such conditions, a test does not absolutely distinguish between the two groups with 100% accuracy. A threshold is selected, above which the test is considered to be “positive” and below which the test is considered to be “negative.” The area under the ROC curve (AUC) provides the C-statistic, which is a measure of the probability that the perceived measurement will allow correct identification of a condition (see, for example, Hanley et al, Radiology 143: 29-36 (1982)). The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest. ROC curves are useful for plotting the performance of a particular feature in distinguishing or discriminating between two populations. Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features (e.g., one or more other epigenetic markers), in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data, or to the Wilcoxon test of ranks.

As used herein, “level” with reference to a SNV or metric refers to the number, percentage, amount or ratio of SNV or metric.

As used herein, a “metric” refers to a number, percentage, ratio and/or type of a single nucleotide variant (SNV). The metrics of the present disclosure are associated with, reflective of or indicative of the number, percentage or ratio of particular SNVs, such as SNVs in the coding region of a nucleic acid molecule; SNVs in the non-coding region of a nucleic acid molecule; SNVs in both the coding and non-coding region of a nucleic acid molecule; SNVs where the coding context of the SNV has been assessed; SNVs that have been determined to be transitions or transversions; SNVs that have been determined to be synonymous or non-synonymous; SNVs resulting from or associated with strand bias; SNVs in which an adenine and thymine, and/or a guanine and cytidine have been targeted; SNVs present in specific motifs (e.g. deaminase or three-mer motifs); and SNVs whether present in motifs or not (i.e. motif-independent metric group). In some examples, the metrics are genetic indicators of deaminase activity.

As used herein, an “SNV type” refers to the specific nucleotide substitution that comprises the SNV, and is selected from among C to T, C to A, C to G, G to T, G to A, G to C, A to T, A to C, A to G, T to A, T to C and T to G SNVs. Thus, for example, a C to T SNV refers to an SNV in which the targeted nucleotide C is replaced with the substituting nucleotide T.

The “nucleic acid” as used herein designates DNA, cDNA, mRNA, RNA, rRNA or cRNA. The term typically refers to polynucleotides greater than 30 nucleotide residues in length.

As used herein, a “predetermined range interval” refers to a range of values, with an upper and lower limit, for a metric that represents a “normal” range of values for the metric. The predetermined range interval can be determined by assessing a metric in two or more healthy subjects. A range interval is then calculated to set the upper and lower limits of what would be considered normal values for that metric. In a particular example, the range interval is calculated by measuring the average plus or minus n standard deviations, whereby the lower limit of the range interval is the average minus n standard deviations and the upper limit of the range interval is the average plus n standard deviations. In still further examples, the upper and lower limits of the predetermined range interval are established using receiver operating characteristic (ROC) curves. The subjects used to determine the predetermined range interval can be of any age, sex or background, or may be of a particular age, sex, ethnic background or other subpopulation. Thus, in some embodiments, two or more range intervals can be calculated for the same metric, whereby each range interval is specific for a particular subpopulation, e.g. a particular sex, age group, ethnic background and/or other subpopulation. The predetermined range interval can be determined using any technique know to those skilled in the art, including manual methods of calculation, an algorithm, a neural network, a support vector machine, deep learning, logistic regression with linear models, machine learning, artificial intelligence and/or a Bayesian network.

As used herein, a “cut-off” with reference to a metric refers to an upper or lower limit of a value for a metric, above or below which represents a “normal” range of values for the metric. The cut-off can be determined by assessing a metric in two or more healthy subjects. A cut-off is then calculated to set an upper or lower limits of what would be considered normal values for that metric. In a particular example, the cut-off is calculated by measuring the average plus or minus n standard deviations, whereby a lower limit cut-off is the average minus n standard deviations and an upper limit cut-off is the average plus n standard deviations. In still further examples, the cut-offs are established using receiver operating characteristic (ROC) curves. The subjects used to determine the cut-off can be of any age, sex or background, or may be of a particular age, sex, ethnic background or other subpopulation. Thus, in some embodiments, two or more cut-offs can be calculated for the same metric, whereby each cut-off is specific for a particular subpopulation, e.g. a particular sex, age group, ethnic background and/or other subpopulation. The cut-off can be determined using any technique know to those skilled in the art, including manual methods of calculation, an algorithm, a neural network, a support vector machine, deep learning, logistic regression with linear models, machine learning, artificial intelligence and/or a Bayesian network.

The term “sensitivity”, as used herein, refers to the probability that a predictive method or kit of the present disclosure gives a positive result when the biological sample is positive, e.g., having the predicted diagnosis. Sensitivity is calculated as the number of true positive results divided by the sum of the true positives and false negatives. Sensitivity essentially is a measure of how well the present disclosure correctly identifies those who have the predicted diagnosis from those who do not have the predicted diagnosis. The statistical methods and models can be selected such that the sensitivity is at least about 60%, and can be, e.g., at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

As used herein, “single nucleotide variant” refers to a variation occurring in the sequence of a nucleic acid molecule (e.g. a subject nucleic acid molecule) compared to another nucleic acid molecule (e.g. a reference nucleic acid molecule or sequence), wherein the variation is a difference in the identity of a single nucleotide (e.g. A, T, C or G).

The terms “subject”, “individual” or “patient”, used interchangeably herein, refer to any animal subject, particularly a mammalian subject. By way of an illustrative example, suitable subjects are humans.

The terms “treat” and “treating” as used herein, unless otherwise indicated, refer to both therapeutic treatment and prophylactic or preventative measures, wherein the object is to inhibit, either partially or completely, ameliorate or slow down (lessen) one or more symptom associated with a disorder or condition, e.g. a neurodegenerative disorder. The term “treatment” as used herein, unless otherwise indicated, refers to the act of treating.

As used herein, the term “treatment regimen” refers to a therapeutic regimen (i.e., after the diagnosis of a neurodegerative disease). The term “treatment regimen” encompasses natural substances and pharmaceutical agents as well as any other treatment regimen.

TABLE A
Nucleotide Symbols

| | |
|---|---|
| A | Adenine |
| C | Cytosine |
| G | Guanine |
| T | Thymine |
| U | Uracil |
| R | Purine - A or G |
| Y | Pyrimidine - C or T |
| S | G or C |
| W | A or T |
| K | G or T |
| M | A or C |
| B | C or G or T |
| D | A or G or T |
| H | A or C or T |
| V | A or C or G |
| N | any base |
| - | gap |

### 2. Metrics

As described herein, SNVs identified in a nucleic acid molecule can be used to determine a plurality of metrics, which can then in turn be used to help distinguish subjects that are likely to have or to develop a neurodegenerative disease from subjects that are unlikely to have or to develop a neurodegenerative disease. As will be appreciated from the description below, the metrics are determined based on the number or percentage of SNVs in any one or more regions of the nucleic acid molecules, and can include an assessment of the targeted nucleotide (i.e. whether the targeted nucleotide is an A, T, C or G), the type of SNV (e.g. whether the targeted nucleotide is now an A, T, G or C), whether the SNV is a transition or transversion SNV and/or whether the SNV is synonymous or non-synonymous, the motif in which the targeted nucleotide resides, the codon context of the SNV, and/or the strand on which the SNV occurs. Any single SNV can therefore be used to generate one or more metrics, and multiple SNVs can be used to generate two more metrics, and typically at least 10, 20, 30, 40, 50, 60, 70, 80, 90, 100 or more metrics. A profile can be built based upon this plurality of metrics, whereupon subjects that are likely to have or to develop a neurodegenerative disease typically have a different profile to subjects that are unlikely to have or to develop a neurodegenerative disease.

As will be apparent from the disclosure herein, the metrics can be associated with or indicative of deaminase activity, i.e. the metrics reflect a number, percentage, ratio and/or type of SNV that may be indicative of the activity of one or more endogenous deaminases, e.g. ADAR, AID or an APOBEC deaminase. In such instances, the metrics may be referred to as genetic indicators of deaminase activity.

Any one or more of the metrics can be assessed for the methods of the present disclosure. Typically, multiple metrics are assessed, such as at least 4, 5, 6, 7, 8, 9, 10, 11, 12, 13, 14, 15, 20, 40, 60, 80, 100 or more.

2.1 Motifs

In instances where the metrics are determined using SNVs identified within a particular motif (i.e. metrics in the motif metric group), motifs may be analysed in pairs: the forward motif and the equivalent reverse complement motif. For example, a forward motif ACG represents a motif in which the underlined C is targeted (or modified or mutated), and the reverse motif is CGT, where the underlined G is targeted (or modified or mutated). As would be understood, identifying a reverse compliment motif is equivalent to identifying the forward motif on the reverse compliment DNA strand. For purposes herein, an underlined nucleotide in a motif is the nucleotide that is targeted (or modified or mutated). In other instances throughout this disclosure, the targeted (or modified or mutated) nucleotide in the motif is denoted by dashes on either side, e.g. ACG or A-C-G indicates that C is targeted (or modified or mutated), while AAA or -A-AA indicates that the 5′ A is targeted (or modified or mutated).

Motifs include those that are known or suggested deaminase motifs. Thus, the metrics may be associated with SNVs in one or more deaminase motifs. Such metrics can therefore also be referred to as genetic indicators of deaminase activity.

Table B sets forth exemplary deaminase motifs, which can be used to generate the metrics of the disclosure. The primary motif for AID is WKC/GYW and there are six secondary motifs (b-g). The primary motif for ADAR is WA/TW, and there are nine secondary motifs (b-j). The primary motif for APOBEC3G (A3G) is CC/GG, and there are eight secondary motifs (b-i). The primary motif for APOBEC3B (A3B) is TCW/WGA, and there are seven secondary motifs (b-i). The motif for APOBEC3F (A3F) is TC/GA and the motif for APOBEC1 (A1) is CA/TG. Thus, reference to a “primary motif” herein is reference to any one of WKC/GYW, WA/TW, CC/GG, and TCW/WGA (i.e. the first four motifs in Table B below). Any SNV that is not at a primary motif, is considered as an “other” SNV (i.e. “other” SNVs include any SNV that is not at one of the four primary motifs, including SNVs that are not at any motif and SNVs that are at secondary or other motifs).

TABLE B
Exemplary deaminase motifs

| Motif Name | Forward Motif | | | | | | | | Reverse Compliment Motif | | | | | | |
|---|---|---|---|---|---|---|---|---|---|---|---|---|---|---|---|
| AID | | W | R | C | | | | / | | | | G | Y | W | |
| ADAR | | | W | A | | | | / | | | | T | W | | |
| A3G | | | C | C | | | | / | | | | G | G | | |
| A3B | | | T | C | W | | | / | | | W | G | A | | |
| AIDb | | W | R | C | G | | | / | | | C | G | Y | W | |
| AIDc | | W | R | C | G | S | | / | | S | C | G | Y | W | |
| AIDd | | W | R | C | Y | | | / | | | R | G | Y | W | |
| AIDe | | W | R | C | G | W | | / | | W | C | G | Y | W | |
| AIDf | | W | R | C | R | | | / | | | Y | G | Y | W | |
| AIDg | | A | G | C | T | N | T | / | A | N | A | G | C | T | |
| ADARb | | | W | A | Y | | | / | | | R | T | W | | |
| ADARc | | S | W | A | Y | | | / | | | R | T | W | S | |
| ADARd | | C | W | A | Y | | | / | | | R | T | W | G | |
| ADARe | | C | W | A | A | | | / | | | T | T | W | G | |
| ADARf | | S | W | A | | | | / | | | | T | W | S | |
| ADARg | | | W | A | A | | | / | | | T | T | W | | |
| ADARh | | | W | A | S | | | / | | | S | T | W | | |
| ADARi | R | A | W | A | | | | / | | | | T | W | T | Y |
| ADARj | | | S | A | R | A | | / | | T | Y | T | S | | |
| A3Gb | | | | C | G | | | / | | | C | G | | | |
| A3Gc | | | C | C | G | W | | / | | W | C | G | G | | |
| A3Gd | | S | C | C | G | W | | / | | W | C | G | G | S | |
| A3Ge | | S | C | C | G | S | | / | | S | C | G | G | S | |
| A3Gf | | S | C | C | G | | | / | | | C | G | G | S | |
| A3Gg | | | C | C | G | S | | / | | S | C | G | G | | |
| A3Gh | | | S | C | G | S | | / | | S | C | G | S | | |
| A3Gi | | S | G | C | G | | | / | | | C | G | C | S | |
| A3Bb | | | T | C | A | | | / | | | T | G | A | | |
| A3Bc | | | T | C | W | A | | / | | T | W | G | A | | |
| A3Bd | | R | T | C | A | | | / | | | T | G | A | Y | |
| A3Be | | Y | T | C | A | | | / | | | T | G | A | R | |
| A3Bf | | S | T | C | G | | | / | | | C | G | A | S | |
| A3Bg | | | T | C | G | A | | / | | T | C | G | A | | |
| A3Bh | | W | T | C | G | | | / | | | C | G | A | W | |
| A3F | | | T | C | | | | / | | | | G | A | | |
| A1 | | | | C | A | | | / | | | T | G | | | |

In further examples, the motifs are not necessarily deaminase motifs. Included among such motifs are general three-mer motifs in which a SNV is detected in one of the positions in the three-mer: M1, M2 or M3. For the purposes herein, typically the targeted nucleotide is an A or C, which may represent a deamination event (although does not necessarily do so). For example, the motif M1 M2 M3 represents a motif in which the targeted (underlined) nucleotide at position M1 is A or C, and the nucleotides at positions M2 and M3 are each independently A, T, G or C. The motif M1 M2 M3 represents a motif in which the targeted (underlined) nucleotide at position M2 is A or C, and the nucleotides at non-targeted positions M1 and M3 are each independently A, T, G or C. The motif M1 M2 M3 represents a motif in which the targeted (underlined) nucleotide at position M3 is A or C, and the nucleotides at non-targeted positions M1 and M2 are each independently A, T, G or C. Thus, there are ninety-six (96) possible three-mer forward motifs of this type, with each motif being associated with the corresponding reverse compliment motif. In further embodiments, metrics can be determined using such three-mer motifs but with the nucleotides at the non-targeted positions being any one of A, T, C, G, R, Y, S, W, K, M or N, resulting in 726 possible motifs.

Non-limiting examples of three-mer motifs include those set forth in Table C below.

TABLE C
Exemplary three-mer motifs

| Motif | Forward | | | | | | Reverse | | | | |
|---|---|---|---|---|---|---|---|---|---|---|---|
| Name | Motif | | | | | | Compliment Motif | | | | |
| Gen2_ACA | | A | C | A | | / | | T | G | T | |
| Gen2_TCA | | T | C | A | | / | | T | G | A | |
| Gen2_CCA | | C | C | A | | / | | T | G | G | |
| Gen2_GCA | | G | C | A | | / | | T | G | C | |
| Gen2_ACT | | A | C | T | | / | | A | G | T | |
| Gen2_TCT | | T | C | T | | / | | A | G | A | |
| Gen2_CCT | | C | C | T | | / | | A | G | G | |
| Gen2_GCT | | G | C | T | | / | | A | G | C | |
| Gen2_ACC | | A | C | C | | / | | G | G | T | |
| Gen2_TCC | | T | C | C | | / | | G | G | A | |
| Gen2_CCC | | C | C | C | | / | | G | G | G | |
| Gen2_GCC | | G | C | C | | / | | G | G | C | |
| Gen2_ACG | | A | C | G | | / | | C | G | T | |
| Gen2_TCG | | T | C | G | | / | | C | G | A | |
| Gen2_CCG | | C | C | G | | / | | C | G | G | |
| Gen2_GCG | | G | C | G | | / | | C | G | C | |
| ADAR_Gen2_AAA | | A | A | A | | / | | T | T | T | |
| ADAR_Gen2_TAA | | T | A | A | | / | | T | T | A | |
| ADAR_Gen2_CAA | | C | A | A | | / | | T | T | G | |
| ADAR_Gen2_GAA | | G | A | A | | / | | T | T | C | |
| ADAR_Gen2_AAT | | A | A | T | | / | | A | T | T | |
| ADAR_Gen2_TAT | | T | A | T | | / | | A | T | A | |
| ADAR_Gen2_CAT | | C | A | T | | / | | A | T | G | |
| ADAR_Gen2_GAT | | G | A | T | | / | | A | T | C | |
| ADAR_Gen2_AAC | | A | A | C | | / | | G | T | T | |
| ADAR_Gen2_TAC | | T | A | C | | / | | G | T | A | |
| ADAR_Gen2_CAC | | C | A | C | | / | | G | T | G | |
| ADAR_Gen2_GAC | | G | A | C | | / | | G | T | C | |
| ADAR_Gen2_AAG | | A | A | G | | / | | C | T | T | |
| ADAR_Gen2_TAG | | T | A | G | | / | | C | T | A | |
| ADAR_Gen2_CAG | | C | A | G | | / | | C | T | G | |
| ADAR_Gen2_GAG | | G | A | G | | / | | C | T | C | |
| ADAR_Gen1_AAA | | | A | A | A | / | T | T | T | | |
| ADAR_Gen1_AAT | | | A | A | T | / | A | T | T | | |
| ADAR_Gen1_AAC | | | A | A | C | / | G | T | T | | |
| ADAR_Gen1_AAG | | | A | A | G | / | C | T | T | | |
| ADAR_Gen1_ATA | | | A | T | A | / | T | A | T | | |
| ADAR_Gen1_ATT | | | A | T | T | / | A | A | T | | |
| ADAR_Gen1_ATC | | | A | T | C | / | G | A | T | | |
| ADAR_Gen1_ATG | | | A | T | G | / | C | A | T | | |
| ADAR_Gen1_ACA | | | A | C | A | / | T | G | T | | |
| ADAR_Gen1_ACT | | | A | C | T | / | A | G | T | | |
| ADAR_Gen1_ACC | | | A | C | C | / | G | G | T | | |
| ADAR_Gen1_ACG | | | A | C | G | / | C | G | T | | |
| ADAR_Gen1_AGA | | | A | G | A | / | T | C | T | | |
| ADAR_Gen1_AGT | | | A | G | T | / | A | C | T | | |
| ADAR_Gen1_AGC | | | A | G | C | / | G | C | T | | |
| ADAR_Gen1_AGG | | | A | G | G | / | C | C | T | | |
| ADAR_Gen3_AAA | A | A | A | | | / | | | T | T | T |
| ADAR_Gen3_ATA | A | T | A | | | / | | | T | A | T |
| ADAR_Gen3_ACA | A | C | A | | | / | | | T | G | T |
| ADAR_Gen3_AGA | A | G | A | | | / | | | T | C | T |
| ADAR_Gen3_TAA | T | A | A | | | / | | | T | T | A |
| ADAR_Gen3_TTA | T | T | A | | | / | | | T | A | A |
| ADAR_Gen3_TCA | T | C | A | | | / | | | T | G | A |
| ADAR_Gen3_TGA | T | G | A | | | / | | | T | C | A |
| ADAR_Gen3_CAA | C | A | A | | | / | | | T | T | G |
| ADAR_Gen3_CTA | C | T | A | | | / | | | T | A | G |
| ADAR_Gen3_CCA | C | C | A | | | / | | | T | G | G |
| ADAR_Gen3_CGA | C | G | A | | | / | | | T | C | G |
| ADAR_Gen3_GAA | G | A | A | | | / | | | T | T | C |
| ADAR_Gen3_GTA | G | T | A | | | / | | | T | A | C |
| ADAR_Gen3_GCA | G | C | A | | | / | | | T | G | C |
| ADAR_Gen3_GGA | G | G | A | | | / | | | T | C | C |
| Gen1_CAA | | | C | A | A | / | T | T | G | | |
| Gen1_CTA | | | C | T | A | / | T | A | G | | |
| Gen1_CCA | | | C | C | A | / | T | G | G | | |
| Gen1_CGA | | | C | G | A | / | T | C | G | | |
| Gen1_CAT | | | C | A | T | / | A | T | G | | |
| Gen1_CTT | | | C | T | T | / | A | A | G | | |
| Gen1_CCT | | | C | C | T | / | A | G | G | | |
| Gen1_CGT | | | C | G | T | / | A | C | G | | |
| Gen1_CAC | | | C | A | C | / | G | T | G | | |
| Gen1_CTC | | | C | T | C | / | G | A | G | | |
| Gen1_CCC | | | C | C | C | / | G | G | G | | |
| Gen1_CGC | | | C | G | C | / | G | C | G | | |
| Gen1_CAG | | | C | A | G | / | C | T | G | | |
| Gen1_CTG | | | C | T | G | / | C | A | G | | |
| Gen1_CCG | | | C | C | G | / | C | G | G | | |
| Gen1_CGG | | | C | G | G | / | C | C | G | | |
| Gen3_AAC | A | A | C | | | / | | | G | T | T |
| Gen3_ATC | A | T | C | | | / | | | G | A | T |
| Gen3_ACC | A | C | C | | | / | | | G | G | T |
| Gen3_AGC | A | G | C | | | / | | | G | C | T |
| Gen3_TAC | T | A | C | | | / | | | G | T | A |
| Gen3_TTC | T | T | C | | | / | | | G | A | A |
| Gen3_TCC | T | C | C | | | / | | | G | G | A |
| Gen3_TGC | T | G | C | | | / | | | G | C | A |
| Gen3_CAC | C | A | C | | | / | | | G | T | G |
| Gen3_CTC | C | T | C | | | / | | | G | A | G |
| Gen3_CCC | C | C | C | | | / | | | G | G | G |
| Gen3_CGC | C | G | C | | | / | | | G | C | G |
| Gen3_GAC | G | A | C | | | / | | | G | T | C |
| Gen3_GTC | G | T | C | | | / | | | G | A | C |
| Gen3_GCC | G | C | C | | | / | | | G | G | C |
| Gen3_GGC | G | G | C | | | / | | | G | C | C |

The motif metrics may reflect (and thus be generated by assessing) the number or percentage of total SNVs in the nucleic acid molecules that are at a particular motif. In further embodiments, motif metrics can be generated by detecting, and can therefore indicate, the particular type of SNV at the targeted nucleotide, e.g. whether there is an A, C or T substituting a targeted G. Further, the metrics can indicate whether the targeted nucleotide is at any position within the codon (i.e. at MC-1, MC-2 or MC-3, as described below). Thus, in some examples, motif metrics can represent a number, percentage or ratio of any SNV at a targeted position in a motif (e.g. a deaminase motif), wherein the targeted nucleotide is at any position within the codon. The percentage of SNVs at the motif is therefore calculated by dividing the total number of SNVs at the motif (regardless of the type of the mutation or codon context of the mutation) by the total number of SNVs in nucleic acid molecule. In other examples, however, only SNVs that are particular types of SNV, such as transition SNVs (i.e. C>T, G>A, T>C and A>G), at a motif are considered in the assessment and metric reflects the percentage, number or ratio of such SNVs. In still further embodiments, both the codon context and the type of SNV is assessed, as described below.

2.2 Codon Context

Mutagens, including deaminases, can target nucleotides in a codon context manner (as described in, for example, WO 2014/066955 and Lindley et al. (2016) Cancer Med. 2016 September; 5(9): 2629-2640). Specifically, mutagenesis can occur at a targeted nucleotide, wherein the targeted nucleotide is present at a particular position within a codon. For the purposes of the present disclosure, the nucleotide positions within an affected codon (MC; i.e., a codon containing the SNV) are annotated MC-1, MC-2 and MC-3, and refer to the first, second and third nucleotide positions, respectively, of the codon when the sequence of the codon is read 5′ to 3′.

Metrics of the present disclosure can be based, at least in part, on a determination of the codon context of an SNV, i.e. whether the SNV is at the first, second or third position in the affected codon, i.e. the MC-1, MC-2 or MC-3 site. As noted above, many deaminases have a preference for targeting nucleotides at a particular position within the affected codon. As such, the number and/or percentage of SNVs that occur at a MC-1, MC-2 or MC-3 site can be a genetic indicator of deaminase activity. As would be appreciated, codon-context metrics are only assessed in the coding region of the nucleic acid molecule.

Metrics based on an assessment of the codon context of an SNV can be motif-independent (i.e. an assessment of the number and/or percentage of SNVs at a particular codon regardless of whether or not the targeted nucleotide is within a particular motif). Thus, these metrics include the number and/or percentage of total SNVs that occur at a MC-1 site; the number and/or percentage of total SNVs that occur at a MC-2 site; and or the number and/or percentage of total SNVs that occur at a MC-3 site.

In other embodiments, a simultaneous assessment of whether the SNV is at a motif, such as a deaminase motif, three-mer motif or five-mer motif (as described above) is also made. Thus, the metrics include codon-context, motif-dependent metrics that are based on the number and/or percentage of SNVs within in a particular motif and at a MC-1 site, MC-2 site and/or MC-3 site. Where the motifs are deaminase motifs, the metrics can be considered as genetic indicators of deaminase activity, and include the number and/or percentage of SNVs that are attributable to a particular motif at a MC-1 site, MC-2 site and/or MC-3 site, such as the number and/or percentage of SNVs that are attributable to AID (i.e. that are at an AID motif) and that occur at a MC-1 site, MC-2 site and/or MC-3 site; the number and/or percentage of SNVs that are attributable to ADAR (i.e. that are at an ADAR motif) and that occur at a MC-1 site, a MC-2 site and/or a MC-3 site; the number and/or percentage of SNVs that are attributable to an APOBEC deaminase (i.e. that are at an APOBEC motif, such as a APOBEC1, APOBEC3A, APOBEC3B, APOBEC3C, APOBEC3D, APOBEC3F, APOBEC3G or APOBEC3H motif) and that occur at a MC-1 site, MC-2 site and/or a MC-3 site.

The codon-context metrics also include those that take into account not only the codon context, but also the nucleotide that is targeted. Thus, the metrics include the number or percentage of SNVs resulting from an adenine which are at the MC1 position, MC2 position and/or MC3 position. For example, the number of SNVs resulting from an adenine may be determined, and the percentage of these that are at a MC-1 site, MC-2 site and/or MC-3 site is then determined to generate the metric. Similarly, the number or percentage of SNVs resulting from a thymine that occurred at the MC1 position, the MC2 position and/or the MC3 position; the number or percentage of SNVs resulting from a cytosine that occurred at the MC1 position, the MC2 position, and/or the MC3 position; the number or percentage of SNVs resulting from a guanine that occurred at the MC1 position, the MC2 position, and/or the MC3 position can be assessed to generate the metrics.

In further embodiments, both the type of SNV (e.g. C>A, C>T, C>G, G>C, G>T, G>A, A>T, A>G, A>C, T>A, T>C or T>G) and the codon context of the SNV is assessed, so as to determine the number or percentage of a particular type of SNV at a MC-1, MC-2 or MC-3 site. Again, in some embodiments, this is performed without a simultaneous assessment of whether the SNV is at a motif associated with a particular deaminase. Thus, metrics include, for example, the number or percentage of C>T SNVs at the MC1 site (typically indicative of AID, APOBEC3B or APOBEC3G activity); the number or percentage of C>T SNVs at the MC2 site (typically indicative of AID, APOBEC3B or APOBEC3G activity); the number or percentage of C>T SNVs at the MC3 site (typically indicative of AID, APOBEC3B or APOBEC3G activity); the number or percentage of G>A SNVs at the MC1 site (typically indicative of AID, APOBEC3B or APOBEC3G activity); the number or percentage of G>A SNVs at the MC2 site (typically indicative of AID, APOBEC3B or APOBEC3G activity); the number or percentage of G>A SNVs at the MC3 site (typically indicative of AID, APOBEC3B or APOBEC3G activity); the number or percentage of T>C SNVs at the MC1 site (typically indicative of ADAR activity); the number or percentage of T>C SNVs at the MC2 site (typically indicative of ADAR activity); the number or percentage of T>C SNVs at the MC3 site (typically indicative of ADAR activity); the number or percentage of A>G SNVs at the MC1 site (typically indicative of ADAR activity); the number or percentage of A>G SNVs at the MC2 site (typically indicative of ADAR activity); and the number or percentage of A>G SNVs at the MC3 site (typically indicative of ADAR activity).

In other embodiments, an assessment of whether the SNV is at a motif (e.g. a deaminase or three-mer), what type of SNV is identified, and also the codon context of the SNV is made to generate the codon context metric.

2.3 Transitions/Transversions

Transitions (Ti) are defined as any variant of a purine to a purine, or a pyrimidine to a pyrimidine (i.e. C>A, G>T, A>C and T>G, and transversions (Tv) are defined as any variant of a pyrimidine to a purine or purine to a pyrimidine (i.e. C>T, C>G, G>A, G>C, A>G, A>T, T>C and T>A). Metrics determined from or associated with SNVs that are transitions or transversions can thus be determined, and include, for example, the number or percentage of SNVs that are transitions or transversions, or the ratio of transitions to transversions or transversions to transitions). In some embodiments, the motif, codon context and/or specific SNV type is also assessed.

2.4 Strand Specificity

Metrics of the present disclosure can also include those based on SNVs identified on just one strand of DNA, i.e. the non-transcribed (or sense or coding) strand or the transcribed (or antisense or template) strand (or “C” or “G” strand, respectively, when SNVs of/from C or G are assessed; or “A” or “T” strand, respectively, when SNVs of/from A or T are assessed. These strand specific metrics typically include an assessment of the number or percentage of SNVs from (or of) a particular targeted nucleotide (e.g. A, T, C or G) on a given strand. Given that particular deaminases can have a preference for targeting a particular nucleotide in a nucleic acid molecule, such metrics can be considered genetic indicators of deaminase activity. For example, adenines are often the target of ADAR, while cytosines are often the target of AID or APOBEC deaminases. Thus, metrics can represent the number or percentage of SNVs resulting from an adenine nucleotide (e.g. detecting the total number of SNVs of A>C, A>T and A>G and expressing this total as a percentage of the total number of SNVs detected); the number or percentage of SNVs resulting from a thymine nucleotide (e.g. detecting the total number of SNVs of T>C, T>A and T>G and expressing this total as a percentage of the total number of SNVs detected); the number or percentage of SNVs resulting from a cytosine nucleotide (e.g. detecting the total number of SNVs of C>A, C>T and C>G and expressing this total as a percentage of the total number of SNVs detected); and/or the number or percentage of SNVs resulting from a guanine nucleotide (e.g. detecting the total number of SNVs of G>C, G>T and G>A and expressing this total as a percentage of the total number of SNVs detected). These can also be an indication of strand bias, as they can show an imbalance in the total number of SNVs of A, T, G or C nucleotides. In a further example, the nucleotide to which the targeted nucleotide becomes is also assessed. For example, the metric may represent the number or percentage of all SNVs that target A that are A>C SNVs.

2.5 AT and GC SNVs

Metrics can also include an assessment of combined SNVs targeting adenine and thymine (AT) and/or combined SNVs targeting guanine and cytosine (GC). The number and/or percentage of SNVs at AT or GC can be assessed. In further instances, a ratio is calculated, such as a ratio of the number or percentage of SNVs that include an adenine or a thymine nucleotide to the number or percentage of SNVs that include a cytosine or a guanine nucleotide (AT:GC ratio) is determined. In further instances, the codon context of the AT or GC SNVs can be taken into consideration to generate the metrics.

2.6 Exemplary Metrics

2.6.1 Coding Region Metrics

Metrics can be determined using SNVs identified in just the coding region (also referred to as the coding sequence or CDS) of a nucleic acid molecule. Exemplary coding region metrics include the mostly motif-associated metrics provided in Table D (with the exception of “CDS variants” which represents the total number of SNVs in the coding region) and the motif-independent metrics provided in Table E. These tables provide the metric name, a brief description of what the metric represents, and how the metric was calculated/determined. Reference to “motif” in the table refers to any one of the motifs described above in section 3.1, including any one of the deaminase or three-mer motifs. Reference to “hits” means “variants”. Some metrics provided in Table D are utilized in the alternative. For example, where a motif comprises a C or G at the targeted nucleotide, the metric that assesses SNVs at these G or C nucleotides is used, and where a motif comprises an A or T at the targeted nucleotide, the alternative metric that assesses SNVs at these A or T nucleotides is used (i.e. the metrics in italics). Thus, where the definition in Table D refers to “motif”, it is the motif that is noted in the metric name (e.g. the metric name in Tables 2-6) and in the associated “motif” column, and “motif SNVs” means the SNVs at that particular motif. For example, “cds:ADAR_W-A-A>G at MC3%” is the percentage of A>G SNVs at the W-A-motif that are at MC3, i.e. of all of A>G SNVs at the W-A-motif, the percentage that are at MC3. Reference to “motif” in the definition column of any of the tables presented herein therefore means the motif referred to in the metric name. For example, the definition “% of motif variants that are at MC3” for the “cds:3Gen2_C-C-C MC3%” metric means the percentage of CCC (or C-C-C) or the reverse complement GGG (G-G-G) variants (or variants at the C-C-C/G-G-G motif) that are at MC3. Reference to “cds” in the metric name indicates that it is the SNVs in the CDS that are assessed for this metric, as expected for a metric that involves an assessment of codon context. In another example, “cds:Gen3_TGC C non-syn %” is the percentage of SNVs at the TGC/GCA (TG-C-/-G-CA) motif in the cds that correspond to (or are) non-synonymous changes. In a further example, cds:A3G_C-C-G>T % refers to the percentage of “G motif SNVs” (i.e. SNVs at “G” on the reverse strand at the -G-G motif) that are G>T mutations. Any SNV that is not at a primary motif, is considered as an “other” SNV (i.e. “other” SNVs include any SNV that is not at one of the four primary motifs, including SNVs that are not at any motif and SNVs that are at secondary or other motifs). Thus, for example, cds:Other MC3% is the percentage of “other” SNVs in the cds (i.e. SNVs not at a primary motif in the CDS) that are at MC3.

TABLE D
Motif-associated coding region metrics.

| | Metric Name | Description of metric | Calculation of metric |
|---|---|---|---|
| 1 | CDS Variants | Total number of CDS variants (i.e. | #CDS |
| | | total number of SNVs within the coding | |
| | | region of the genome) | |
| 2 | Motif Hits | Number of motif variants (i.e. number | #motif |
| | | of variants at a given motif) | |
| 3 | Motif % | Percentage of motif variants (i.e. | #motif/#CDS |
| | | number of variants at a given motif/ | |
| | | #CDS variants, as a %) | |
| 4 | Motif Ti % | Percentage of motif variants that are | #motif_Ti/#CDS |
| | | transitions (i.e. number of motif | |
| | | variants which are transitions/#CDS | |
| | | variants, as a %) | |
| 5 | Motif MC1 % | % motif variants which are at MC1 | #motif_MC1/#motif |
| 6 | Motif MC2 % | % motif variants which are at MC2 | #motif_MC2/#motif |
| 7 | Motif MC3 % | % motif variants which are at MC3 | #motif_MC3/#motif |
| 8 | Motif C > T at MC1 % | % motif C > T variants which are at | #motif_C > T_MC1/ |
| | | MC1 (of all C > T) | #motif_C > T_all |
| | Motif A > G at MC1 % | % motif A > G variants which are at | #motif_A > G_MC1/ |
| | | MC1 (of all A > G) | #motif_A > G_all |
| 9 | Motif C > T at MC1 | % motif C > T variants which are at | #motif_C > T_MC1/#motif |
| | motif % | MC1 (of all motif variants) | |
| | Motif A > G at MC1 | % motif A > G variants which are at | #motif_A > G_MC1/#motif |
| | motif % | MC1 (of all motif variants) | |
| 10 | Motif C > T at MC1 | % motif C > T variants which are at | #motif_C > T_MC1/#cds |
| | cds % | MC1 (of all cds) | |
| | Motif A > G at MC1 | % motif A > G variants which are at | #motif_A > G_MC1/#cds |
| | cds % | MC1 (of all cds) | |
| 11 | Motif C > T at MC2 % | % motif C > T variants which are at | #motif_C > T_MC2/ |
| | | MC2 (of all C > T) | #motif_C > T_all |
| | Motif A > G at MC2 % | % motif A > G variants which are at | #motif_A > G_MC2/ |
| | | MC2 (of all A > G) | #motif_A > G_all |
| 12 | Motif C > T at MC2 | % motif C > T variants which are at | #motif_C > T_MC2/#motif |
| | motif % | MC2 (of all motif variants) | |
| | Motif A > G at MC2 | % motif A > G variants which are at | #motif_A > G_MC2/#motif |
| | motif % | MC2 (of all motif variants) | |
| 13 | Motif C > T at MC2 | % motif C > T variants which are at | #motif_C > T_MC2/#cds |
| | cds % | MC2 (of all cds) | |
| | Motif A > G at MC2 | % motif A > G variants which are at | #motif_A > G_MC2/#cds |
| | cds % | MC2 (of all cds) | |
| 14 | Motif C > T at MC3 % | % motif C > T variants which are at | #motif_C > T_MC3/ |
| | | MC3 (of all C > T) | #motif_C > T_all |
| | Motif A > G at MC3 % | % motif A > G variants which are at | #motif_A > G_MC3/ |
| | | MC3 (of all A > G) | #motif_A > G_all |
| 15 | Motif C > T at MC3 | % motif C > T variants which are at | #motif_C > T_MC3/#motif |
| | motif % | MC3 (of all motif variants) | |
| | Motif A > G at MC3 | % motif A > G variants which are at | #motif_A > G_MC3/#motif |
| | motif % | MC3 (of all motif variants) | |
| 16 | Motif C > T at MC3 | % motif C > T variants which are at | #motif_C > T_MC3/#cds |
| | cds % | MC3 (of all cds) | |
| | Motif A > G at MC3 | % motif A > G variants which are at | #motif_A > G_MC3/#cds |
| | cds % | MC3 (of all cds) | |
| 17 | Motif G > A at MC1 % | % motif G > A variants which are at | #motif_G > A_MC1/ |
| | | MC1 (of all G > A) | #motif_G > A_all |
| 18 | Motif T > C at MC1 % | % motif T > C variants which are at | #motif_T > C_MC1/ |
| | | MC1 (of all T > C) | #motif_T > C_all |
| 19 | Motif G > A at MC1 | % motif G > A variants which are at | #motif_G > A_MC1/#motif |
| | motif % | MC1 (of all motif variants) | |
| 20 | Motif T > C at MC1 | % motif T > C variants which are at | #motif_T > C_MC1/#motif |
| | motif % | MC1 (of all motif variants) | |
| 21 | Motif G > A at MC1 | % motif G > A variants which are at | #motif_G > A_MC1/#cds |
| | cds % | MC1 (of all cds) | |
| 22 | Motif T > C at MC1 | % motif T > C variants which are at | #motif_T > C_MC1/#cds |
| | cds % | MC1 (of all cds) | |
| 23 | Motif G > A at MC2 % | % motif G > A variants which are at | #motif_G > A_MC2/ |
| | | MC2 (of all G > A) | #motif_G > A_all |
| | Motif T > C at MC2 % | % motif T > C variants which are at | #motif_T > C_MC2/ |
| | | MC2 (of all T > C) | #motif_T > C_all |
| 24 | Motif G > A at MC2 | % motif G > A variants which are at | #motif_G > A_MC2/#motif |
| | motif % | MC2 (of all motif variants) | |
| | Motif T > C at MC2 | % motif T > C variants which are at | #motif_T > C_MC2/#motif |
| | motif % | MC2 (of all motif variants) | |
| 25 | Motif G > A at MC2 | % motif G > A variants which are at | #motif_G > A_MC2/#cds |
| | cds % | MC2 (of all cds) | |
| | Motif T > C at MC2 | % motif T > C variants which are at | #motif_T > C_MC2/#cds |
| | cds % | MC2 (of all cds) | |
| 26 | Motif G > A at MC3 % | % motif G > A variants which are at | #motif_G > A_MC3/ |
| | | MC3 (of all G > A) | #motif_G > A_all |
| | Motif T > C at MC3 % | % motif T > C variants which are at | #motif_T > C_MC3/ |
| | | MC3 (of all T > C) | #motif_T > C_all |
| 27 | Motif G > A at MC3 | % motif G > A variants which are at | #motif_G > A_MC3/#motif |
| | motif % | MC3 (of all motif variants) | |
| | Motif T > C at MC3 | % motif T > C variants which are at | #motif_T > C_MC3/#motif |
| | motif % | MC3 (of all motif variants) | |
| 28 | Motif G > A at MC3 | % motif G > A variants which are at | #motif_G > A_MC3/#cds |
| | cds % | MC3 (of all cds) | |
| | Motif T > C at MC3 | % motif T > C variants which are at | #motif_T > C_MC3/#cds |
| | cds % | MC3 (of all cds) | |
| 29 | Motif C > T % | % motif variants that are C > T/of all C | #motif_C > T/#motif_C |
| | | variants | |
| | Motif A > G % | % motif variants that are A > G/of all | #motif_A > G/#motif_A |
| | | A variants | |
| 30 | Motif C > T motif % | % motif variants that are C > T/of all | #motif_C > T/#motif |
| | | motif variants | |
| | Motif A > G motif % | % motif variants that are A > G/of all | #motif_A > G/#motif |
| | | motif variants | |
| 31 | Motif C > T cds % | % motif variants that are C > T/of all | #motif_C > T/#cds |
| | | CDS variants | |
| | Motif A > G cds % | % motif variants that are A > G/of all | #motif_A > G/#cds |
| | | CDS variants | |
| 32 | Motif C > A % | % motif variants that are C > A/of all C | #motif_C > A/#motif_C |
| | | variants | |
| | Motif A > C % | % motif variants that are A > C/of all A | #motif_A > C/#motif_A |
| | | variants | |
| 33 | Motif C > A motif % | % motif variants that are C > A/of all | #motif_C > A/#motif |
| | | motif variants | |
| | Motif A > C motif % | % motif variants that are A > C/of all | #motif_A > C/#motif |
| | | motif variants | |
| 34 | Motif C > A cds % | % motif variants that are C > A/of all | #motif_C > A/#cds |
| | | CDS variants | |
| | Motif A > C cds % | % motif variants that are A > C/of all | #motif_A > C/#cds |
| | | CDS variants | |
| 35 | Motif C > G % | % motif variants that are C > G/of all | #motif_C > G/#motif_C |
| | | C variants | |
| | Motif A > T % | % motif variants that are A > T/of all A | #motif_A > T/#motif_A |
| | | variants | |
| 36 | Motif C > G motif % | % motif variants that are C > G/of all | #motif_C > G/#motif |
| | | motif variants | |
| | Motif A > T motif % | % motif variants that are A > T/of all | #motif_A > T/#motif |
| | | motif variants | |
| 37 | Motif C > G cds % | % motif variants that are C > G/of all | #motif_C > G/#cds |
| | | CDS variants | |
| | Motif A > T cds % | % motif variants that are A > T/of all | #motif_A > T/#cds |
| | | CDS variants | |
| 38 | Motif G > A % | % motif variants that are G > A/of all | #motif_G > A/#motif_G |
| | | G variants | |
| | Motif T > C % | % motif variants that are T > C/of all T | #motif_T > C/#motif_T |
| | | variants | |
| 39 | Motif G > A motif % | % motif variants that are G > A/of all | #motif_G > A/#motif |
| | | motif variants | |
| | Motif T > C motif % | % motif variants that are T > C/of all | #motif_T > C/#motif |
| | | motif variants | |
| 40 | Motif G > A cds % | % motif variants that are G > A/of all | #motif_G > A/#cds |
| | | CDS variants | |
| | Motif T > C cds % | % motif variants that are T > C/of all | #motif_T > C/#cds |
| | | CDS variants | |
| 41 | Motif G > T % | % motif variants that are G > T/of all | #motif_G > T/#motif_G |
| | | G variants | |
| | Motif T > G % | % motif variants that are T > G/of all T | #motif_T > G/#motif_T |
| | | variants | |
| 42 | Motif G > T motif % | % motif variants that are G > T/of all | #motif_G > T/#motif |
| | | motif variants | |
| | Motif T > G motif % | % motif variants that are T > G/of all | #motif_T > G/#motif |
| | | motif variants | |
| 43 | Motif G > T cds % | % motif variants that are G > T/of all | #motif_G > T/#cds |
| | | CDS variants | |
| | Motif T > G cds % | % motif variants that are T > G/of all | #motif_T > G/#cds |
| | | CDS variants | |
| 44 | Motif G > C % | % motif variants that are G > C/of all | #motif_G > C/#motif_G |
| | | G variants | |
| | Motif T > A % | % motif variants that are T > A/of all T | #motif_T > A/#motif_T |
| | | variants | |
| 45 | Motif G > C motif % | % motif variants that are G > C/of all | #motif_G > C/#motif |
| | | motif variants | |
| | Motif T > A motif % | % motif variants that are T > A/of all | #motif_T > A/#motif |
| | | motif variants | |
| 46 | Motif G > C cds % | % motif variants that are G > C/of all | #motif_G > C/#cds |
| | | CDS variants | |
| | Motif T > A cds % | % motif variants that are T > A/of all | #motif_T > A/#cds |
| | | CDS variants | |
| 47 | Motif Ti/Tv % | % motif variants that are transitions | #motif_Ti/#motif |
| 48 | Motif C:G % | % motif variants that are C - strand | #motif_C/#motif |
| | | bias | |
| | Motif A:T % | % motif variants that are A - strand | #motif_A/#motif |
| | | bias | |
| 49 | Motif Ti C:G % | % motif variants - transition only - | #motif_C > T/#motif_Ti |
| | | that are C - strand bias | |
| | Motif Ti A:T % | % motif variants - transition only - | #motif_A > G/#motif_Ti |
| | | that are A - strand bias | |
| 50 | Motif non-syn % | % motifs variants which are non- | #motif_ns/#motif |
| | | synonymous protein change | |
| 51 | Motif C non-syn % | % motifs variants - C strand only - | #motif_C_ns/#motif |
| | | which are non-synonymous protein | |
| | | change | |
| | Motif A non-syn % | % motifs variants - A strand only - | #motif_A_ns/#motif |
| | | which are non-synonymous protein | |
| | | change | |
| 52 | Motif G non-syn % | % motifs variants - G strand only - | #motif_G_ns/#motif |
| | | which are non-synonymous protein | |
| | | change | |
| | Motif T non-syn % | % motifs variants - T strand only - | #motif_T_ns/#motif |
| | | which are non-synonymous protein | |
| | | change | |
| 53 | Motif MC1 non-syn | % non-syn of motif variants at MC1 | #motif_MC1_ns/#motif_MC1 |
| | % | | |
| 54 | Motif MC2 non-syn | % non-syn of motif variants at MC2 | #motif_MC2_ns/#motif_MC2 |
| | % | | |
| 55 | Motif MC3 non-syn | % non-syn of motif variants at MC2 | #motif_MC3_ns/#motif_MC3 |
| | % | | |
| 56 | Motif C > A at MC1 % | % motif C > A variants which are at | #motif_C > A_MC1/ |
| | | MC1 (of all C > A) | #motif_C > A_all |
| | Motif A > C at MC1 % | % motif A > C variants which are at | #motif_A > C_MC1/ |
| | | MC1 (of all C > A) | #motif_A > C_all |
| 57 | Motif C > A at MC1 | % motif C > A variants which are at | #motif_C > A_MC1/#motif |
| | motif % | MC1 (of all motif variants) | |
| | Motif A > C at MC1 | % motif A > C variants which are at | #motif_A > C_MC1/#motif |
| | motif % | MC1 (of all motif variants) | |
| 58 | Motif C > A at MC1 | % motif C > A variants which are at | #motif_C > A_MC1/#cds |
| | cds % | MC1 (of all cds) | |
| | Motif A > C at MC1 | % motif A > C variants which are at | #motif_A > C_MC1/#cds |
| | cds % | MC1 (of all cds) | |
| 59 | Motif C > A at MC2 % | % motif C > A variants which are at | #motif_C > A_MC2/ |
| | | MC2 | #motif_C > A_all |
| | Motif A > C at MC2 % | % motif A > C variants which are at | #motif_A > C_MC2/ |
| | | MC2 (of all A > C) | #motif_A > C_all |
| 60 | Motif C > A at MC2 | % motif C > A variants which are at | #motif_C > A_MC2/#motif |
| | motif % | MC2 (of all motif variants) | |
| | Motif A > C at MC2 | % motif A > C variants which are at | #motif_A > C_MC2/#motif |
| | motif % | MC2 (of all motif variants) | |
| 61 | Motif C > A at MC2 | % motif C > A variants which are at | #motif_C > A_MC2/#cds |
| | cds % | MC2 (of all cds) | |
| | Motif A > C at MC2 | % motif A > C variants which are at | #motif_A > C_MC2/#cds |
| | cds % | MC2 (of all cds) | |
| 62 | Motif C > A at MC3 % | % motif C > A variants which are at | #motif_C > A_MC3/ |
| | | MC3 | #motif_C > A_all |
| | Motif A > C at MC3 % | % motif A > C variants which are at | #motif_A > C_MC3/ |
| | | MC3 (of all A > C) | #motif_A > C_all |
| 63 | Motif C > A at MC3 | % motif C > A variants which are at | #motif_C > A_MC3/#motif |
| | motif % | MC3 (of all motif variants) | |
| | Motif A > C at MC3 | % motif A > C variants which are at | #motif_A > C_MC3/#motif |
| | motif % | MC3 (of all motif variants) | |
| 64 | Motif C > A at MC3 | % motif C > A variants which are at | #motif_C > A_MC3/#cds |
| | cds % | MC3 (of all cds) | |
| | Motif A > C at MC3 | % motif A > C variants which are at | #motif_A > C_MC3/#cds |
| | cds % | MC3 (of all cds) | |
| 65 | Motif G > T at MC1 % | % motif G > T variants which are at | #motif_G > T_MC1/ |
| | | MC1 (of all G > T) | #motif_G > T_all |
| | Motif T > G at MC1 % | % motif T > G variants which are at | #motif_T > G_MC1/ |
| | | MC1 (of all T > G) | #motif_T > G_all |
| 66 | Motif G > T at MC1 | % motif G > T variants which are at | #motif_G > T_MC1/#motif |
| | motif % | MC1 (of all motif variants) | |
| | Motif T > G at MC1 | % motif T > G variants which are at | #motif_T > G_MC1/#motif |
| | motif % | MC1 (of all motif variants) | |
| 67 | Motif G > T at MC1 | % motif G > T variants which are at | #motif_G > T_MC1/#cds |
| | cds % | MC1 (of all cds) | |
| | Motif T > G at MC1 | % motif T > G variants which are at | #motif_T > G_MC1/#cds |
| | cds % | MC1 (of all cds) | |
| 68 | Motif G > T at MC2 % | % motif G > T variants which are at | #motif_G > T_MC2/ |
| | | MC2 (of all G > T) | #motif_G > T_all |
| | Motif T > G at MC2 % | % motif T > G variants which are at | #motif_T > G_MC2/ |
| | | MC2 (of all T > G) | #motif_T > G_all |
| 69 | Motif G > T at MC2 | % motif G > T variants which are at | #motif_G > T_MC2/#motif |
| | motif % | MC2 (of all motif variants) | |
| | Motif T > G at MC2 | % motif T > G variants which are at | #motif_T > G_MC2/#motif |
| | motif % | MC2 (of all motif variants) | |
| 70 | Motif G > T at MC2 | % motif G > T variants which are at | #motif_G > T_MC2/#cds |
| | cds % | MC2 (of all cds) | |
| | Motif T > G at MC2 | % motif T > G variants which are at | #motif_T > G_MC2/#cds |
| | cds % | MC2 (of all cds) | |
| 71 | Motif G > T at MC3 % | % motif G > T variants which are at | #motif_G > T_MC3/ |
| | | MC3 (of all G > T) | #motif_G > T_all |
| | Motif T > G at MC3 % | % motif T > G variants which are at | #motif_T > G_MC3/ |
| | | MC3 (of all T > G) | #motif_T > G_all |
| 72 | Motif G > T at MC3 | % motif G > T variants which are at | #motif_G > T_MC3/#motif |
| | motif % | MC3 (of all motif variants) | |
| | Motif T > G at MC3 | % motif T > G variants which are at | #motif_T > G_MC3/#motif |
| | motif % | MC3 (of all motif variants) | |
| 73 | Motif G > T at MC3 | % motif G > T variants which are at | #motif_G > T_MC3/#cds |
| | cds % | MC3 (of all cds) | |
| | Motif T > G at MC3 | % motif T > G variants which are at | #motif_T > G_MC3/#cds |
| | cds % | MC3 (of all cds) | |
| 74 | Motif C > G at MC1 % | % motif C > G variants which are at | #motif_C > G_MC1/ |
| | | MC1 (of all C > G) | #motif_C > G_all |
| | Motif A > T at MC1 % | % motif A > T variants which are at | #motif_A > T_MC1/ |
| | | MC1 (of all A > T) | #motif_A > T_all |
| 75 | Motif C > G at MC1 | % motif C > G variants which are at | #motif_C > G_MC1/#motif |
| | motif % | MC1 (of all motif variants) | |
| | Motif A > T at MC1 | % motif A > T variants which are at | #motif_A > T_MC1/#motif |
| | motif % | MC1 (of all motif variants) | |
| 76 | Motif C > G at MC1 | % motif C > G variants which are at | #motif_C > G_MC1/#cds |
| | cds % | MC1 (of all cds) | |
| | Motif A > T at MC1 | % motif A > T variants which are at | #motif_A > T_MC1/#cds |
| | cds % | MC1 (of all cds) | |
| 77 | Motif C > G at MC2 % | % motif C > G variants which are at | #motif_C > G_MC2/ |
| | | MC2 (of all C > G) | #motif_C > G_all |
| | Motif A > T at MC2 % | % motif A > T variants which are at | #motif_A > T_MC2/ |
| | | MC2 (of all A > T) | #motif_A > T_all |
| 78 | Motif C > G at MC2 | % motif C > G variants which are at | #motif_C > G_MC2/#motif |
| | motif % | MC2 (of all motif variants) | |
| | Motif A > T at MC2 | % motif A > T variants which are at | #motif_A > T_MC2/#motif |
| | motif % | MC2 (of all motif variants) | |
| 79 | Motif C > G at MC2 | % motif C > G variants which are at | #motif_C > G_MC2/#cds |
| | cds % | MC2 (of all cds) | |
| | Motif A > T at MC2 | % motif A > T variants which are at | #motif_A > T_MC2/#cds |
| | cds % | MC2 (of all cds) | |
| 80 | Motif C > G at MC3 % | % motif C > G variants which are at | #motif_C > G_MC3/ |
| | | MC3 (of all C > G) | #motif_C > G_all |
| | Motif A > T at MC3 % | % motif A > T variants which are at | #motif_A > T_MC3/ |
| | | MC3 (of all A > T) | #motif_A > T_all |
| 81 | Motif C > G at MC3 | % motif C > G variants which are at | #motif_C > G_MC3/#motif |
| | motif % | MC3 (of all motif variants) | |
| | Motif A > T at MC3 | % motif A > T variants which are at | #motif_A > T_MC3/#motif |
| | motif % | MC3 (of all motif variants) | |
| 82 | Motif C > G at MC3 | % motif C > G variants which are at | #motif_C > G_MC3/#cds |
| | cds % | MC3 (of all cds) | |
| | Motif A > T at MC3 | % motif A > T variants which are at | #motif_A > T_MC3/#cds |
| | cds % | MC3 (of all cds) | |
| 83 | Motif G > C at MC1 % | % motif G > C variants which are at | #motif_G > C_MC1/ |
| | | MC1 (of all G > C) | #motif_G > C_all |
| | Motif T > A at MC1 % | % motif T > A variants which are at | #motif_T > A_MC1/ |
| | | MC1 (of all T > A) | #motif_T > A_all |
| 84 | Motif G > C at MC1 | % motif G > C variants which are at | #motif_G > C_MC1/#motif |
| | motif % | MC1 (of all motif variants) | |
| | Motif T > A at MC1 | % motif T > A variants which are at | #motif_T > A_MC1/#motif |
| | motif % | MC1 (of all motif variants) | |
| 85 | Motif G > C at MC1 | % motif G > C variants which are at | #motif_G > C_MC1/#cds |
| | cds % | MC1 (of all cds) | |
| | Motif T > A at MC1 | % motif T > A variants which are at | #motif_T > A_MC1/#cds |
| | cds % | MC1 (of all cds) | |
| 86 | Motif G > C at MC2 % | % motif G > C variants which are at | #motif_G > C_MC2/ |
| | | MC2 (of all G > C) | #motif_G > C_all |
| | Motif T > A at MC2 % | % motif T > A variants which are at | #motif_T > A_MC2/ |
| | | MC2 (of all T > A) | #motif_T > A_all |
| 87 | Motif G > C at MC2 | % motif G > C variants which are at | #motif_G > C_MC2/#motif |
| | motif % | MC2 (of all motif variants) | |
| | Motif T > A at MC2 | % motif T > A variants which are at | #motif_T > A_MC2/#motif |
| | motif % | MC2 (of all motif variants) | |
| 88 | Motif G > C at MC2 | % motif G > C variants which are at | #motif_G > C_MC2/#cds |
| | cds % | MC2 (of all cds) | |
| | Motif T > A at MC2 | % motif T > A variants which are at | #motif_T > A_MC2/#cds |
| | cds % | MC2 (of all cds) | |
| 89 | Motif G > C at MC3 % | % motif G > C variants which are at | #motif_G > C_MC3/ |
| | | MC3 (of all G > C) | #motif_G > C_all |
| | Motif T > A at MC3 % | % motif T > A variants which are at | #motif_T > A_MC3/ |
| | | MC3 (of all T > A) | #motif_T > A_all |
| 90 | Motif G > C at MC3 | % motif G > C variants which are at | #motif_G > C_MC3/#motif |
| | motif % | MC3 (of all motif variants) | |
| | Motif T > A at MC3 | % motif T > A variants which are at | #motif_T > A_MC3/#motif |
| | motif % | MC3 (of all motif variants) | |
| 91 | Motif G > C at MC3 | % motif G > C variants which are at | #motif_G > C_MC3/#cds |
| | cds % | MC3 (of all cds) | |
| | Motif T > A at MC3 | % motif T > A variants which are at | #motif_T > A_MC3/#cds |
| | cds % | MC3 (of all cds) | |

TABLE E
Motif-independent coding region metrics

| | Metric Name | Description of metric | Calculation of metric |
|---|---|---|---|
| 1 | cds:All A total | Total number of A CDS | #A |
| | | variants (i.e. number of | |
| | | variants in the CDS that are A) | |
| 2 | cds:All T total | Total number of T CDS variants | #T |
| 3 | cds:All C total | Total number of C CDS variants | #C |
| 4 | cds:All G total | Total number of G CDS variants | #G |
| 5 | cds:All A % | number of A variants/#CDS | #A/#CDS |
| | | variants % | |
| 6 | cds:All T % | number of T variants/#CDS | #T/#CDS |
| | | variants % | |
| 7 | cds:All C % | number of C variants/#CDS | #C/#CDS |
| | | variants % | |
| 8 | cds:All G % | number of G variants/#CDS | #G/#CDS |
| | | variants % | |
| 9 | cds:All MC1 % | % CDS variants which are at | #MC1/#CDS |
| | | MC1 | |
| 10 | cds:All MC2 % | % CDS variants which are at | #MC2/#CDS |
| | | MC2 | |
| 11 | cds:All MC3 % | % CDS variants which are at | #MC3/#CDS |
| | | MC3 | |
| 12 | cds:All A MC1 % | % A variants which are at MC1 | #A_MC1/#CDS |
| 13 | cds:All A MC2 % | % A variants which are at MC2 | #A_MC2/#CDS |
| 14 | cds:All A MC3 % | % A variants which are at MC3 | #A_MC3/#CDS |
| 15 | cds:All T MC1 % | % T variants which are at MC1 | #T_MC1/#CDS |
| 16 | cds:All T MC2 % | % T variants which are at MC2 | #T_MC2/#CDS |
| 17 | cds:All T MC3 % | % T variants which are at MC3 | #T_MC3/#CDS |
| 18 | cds:All C MC1 % | % C variants which are at MC1 | #C_MC1/#CDS |
| 19 | cds:All C MC2 % | % C variants which are at MC2 | #C_MC2/#CDS |
| 20 | cds:All C MC3 % | % C variants which are at MC3 | #C_MC3/#CDS |
| 21 | cds:All G MC1 % | % G variants which are at MC1 | #G_MC1/#CDS |
| 22 | cds:All G MC2 % | % G variants which are at MC2 | #G_MC2/#CDS |
| 23 | cds:All G MC3 % | % G variants which are at MC3 | #G_MC3/#CDS |
| 24 | cds:All MC1 A % | % MC1 variants which are A | #A_MC1/#MC1 |
| 25 | cds:All MC1 T % | % MC1 variants which are T | #T_MC1/#MC1 |
| 26 | cds:All MC1 C % | % MC1 variants which are C | #C_MC1/#MC1 |
| 27 | cds:All MC1 G % | % MC1 variants which are G | #G_MC1/#MC1 |
| 28 | cds:All MC2 A % | % MC2 variants which are A | #A_MC2/#MC2 |
| 29 | cds:All MC2 T % | % MC2 variants which are T | #T_MC2/#MC2 |
| 30 | cds:All MC2 C % | % MC2 variants which are C | #C_MC2/#MC2 |
| 31 | cds:All MC2 G % | % MC2 variants which are G | #G_MC2/#MC2 |
| 32 | cds:All MC3 A % | % MC3 variants which are A | #A_MC3/#MC3 |
| 33 | cds:All MC3 T % | % MC3 variants which are T | #T_MC3/#MC3 |
| 34 | cds:All MC3 C % | % MC3 variants which are C | #C_MC3/#MC3 |
| 35 | cds:All MC3 G % | % MC3 variants which are G | #G_MC3/#MC3 |
| 36 | cds:All AT Ti/Tv | % A and T variants that are | (#A_Ti + #T_Ti )/(#A + #T) |
| | % | transitions | |
| 37 | cds:All CG Ti/Tv | % C and G variants that are | (#C_Ti + #G_Ti )/(#C + #G) |
| | % | transitions | |
| 38 | cds:All MC1 Ti/Tv | % MC1 variants that are | #MC1_Ti/#MC1 |
| | % | transitions | |
| 39 | cds:All MC2 Ti/Tv | % MC2 variants that are | #MC2_Ti/#MC2 |
| | % | transitions | |
| 40 | cds:All MC3 Ti/Tv | % MC3 variants that are | #MC3_Ti/#MC3 |
| | % | transitions | |
| 41 | cds:All A MC1 | % A MC1 variants that are | #A_MC1_Ti/#A_MC1 |
| | Ti/Tv % | transitions | |
| 42 | cds:All A MC2 | % A MC2 variants that are | #A_MC2_Ti/#A_MC2 |
| | Ti/Tv % | transitions | |
| 43 | cds:All A MC3 | % A MC3 variants that are | #A_MC3_Ti/#A_MC3 |
| | Ti/Tv % | transitions | |
| 44 | cds:All T MC1 | % T MC1 variants that are | #T_MC1_Ti/#T_MC1 |
| | Ti/Tv % | transitions | |
| 45 | cds:All T MC2 | % T MC2 variants that are | #T_MC2_Ti/#T_MC2 |
| | Ti/Tv % | transitions | |
| 46 | cds:All T MC3 | % T MC3 variants that are | #T_MC3_Ti/#T_MC3 |
| | Ti/Tv % | transitions | |
| 47 | cds:All C MC1 | % C MC1 variants that are | #C_MC1_Ti/#C_MC1 |
| | Ti/Tv % | transitions | |
| 48 | cds:All C MC2 | % C MC2 variants that are | #C_MC2_Ti/#C_MC2 |
| | Ti/Tv % | transitions | |
| 49 | cds:All C MC3 | % C MC3 variants that are | #C_MC3_Ti/#C_MC3 |
| | Ti/Tv % | transitions | |
| 50 | cds:All G MC1 | % G MC1 variants that are | #G_MC1_Ti/#G_MC1 |
| | Ti/Tv % | transitions | |
| 51 | cds:All G MC2 | % G MC2 variants that are | #G_MC2_Ti/#G_MC2 |
| | Ti/Tv % | transitions | |
| 52 | cds:All G MC3 | % G MC3 variants that are | #G_MC3_Ti/#G_MC3 |
| | Ti/Tv % | transitions | |
| 53 | cds:All C:G % | % variants that are C - | #C/(#C + #G) |
| | | compared to G - strand bias % | |
| 54 | cds:All A:T % | % variants that are A - | #A/(#A + #T) |
| | | compared to T - strand bias % | |
| 55 | cds:All AT:GC % | % A or T variants -compared | (#A + #T)/#CDS |
| | | to all variants | |
| 56 | cds:All MC1 C:G % | % MC1 variants that are C - | #C_MC1/(#C_MC1 + #G_MC1) |
| | | compared to G - strand bias % | |
| 57 | cds:All MC2 C:G % | % MC2 variants that are C - | #C_MC2/(#C_MC2 + #G_MC2) |
| | | compared to G - strand bias % | |
| 58 | cds:All MC3 C:G % | % MC3 variants that are C - | #C_MC3/(#C_MC3 + #G_MC3) |
| | | compared to G - strand bias % | |
| 59 | cds:All MC1 A:T % | % MC1 variants that are A - | #A_MC1/(#A_MC1 + #T_MC1) |
| | | compared to T - strand bias % | |
| 60 | cds:All MC2 A:T % | % MC2 variants that are A - | #A_MC2/(#A_MC2 + #T_MC2) |
| | | compared to T - strand bias % | |
| 61 | cds:All MC3 A:T % | % MC3 variants that are A - | #A_MC3/(#A_MC3 + #T_MC3) |
| | | compared to T - strand bias % | |
| 62 | cds:All MC1 AT:GC | % MC1 A or T variants - | (#A_MC1 + #T_MC1)/#CDS_MC1 |
| | % | compared to all variants | |
| 63 | cds:All MC2 AT:GC | % MC2 A or T variants - | (#A_MC2 + #T_MC2)/#CDS_MC2 |
| | % | compared to all variants | |
| 64 | cds:All MC3 AT:GC | % MC3 A or T variants - | (#A_MC2 + #T_MC3)/#CDS_MC3 |
| | % | compared to all variants | |
| 65 | cds:All A > G % | % variants that are A > G/of all | #A > G/#A |
| | | A variants | |
| 66 | cds:All A > C % | % variants that are A > C/of all | #A > C/#A |
| | | A variants | |
| 67 | cds:All A > T % | % variants that are A > T/of all | #A > T/#A |
| | | A variants | |
| 68 | cds:All T > C % | % variants that are T > C/of all | #T > C/#T |
| | | T variants | |
| 69 | cds:All T > G % | % variants that are T > G/of all | #T > G/#T |
| | | T variants | |
| 70 | cds:All T > A % | % variants that are T > A/of all | #T > A/#T |
| | | T variants | |
| 71 | cds:All C > T % | % variants that are C > T/of all | #C > T/#C |
| | | C variants | |
| 72 | cds:All C > A % | % variants that are C > A/of all | #C > A/#C |
| | | C variants | |
| 73 | cds:All C > G % | % variants that are C > G/of all | #C > G/#C |
| | | C variants | |
| 74 | cds:All G > A % | % variants that are G > A/of all | #G > A/#G |
| | | G variants | |
| 75 | cds:All G > T % | % variants that are G > T/of all | #G > T/#G |
| | | G variants | |
| 76 | cds:All G > C % | % variants that are G > C/of all | #G > C/#G |
| | | G variants | |
| 77 | cds:All non-syn % | % variants which are non- | #CDS_ns/#CDS |
| | | synonymous | |
| 78 | cds:All A non-syn | % A variants which are non- | #A_ns/#A |
| | % | synonymous | |
| 79 | cds:All T non-syn | % T variants which are non- | #T_ns/#T |
| | % | synonymous | |
| 80 | cds:All C non-syn | % C variants which are non- | #C_ns/#C |
| | % | synonymous | |
| 81 | cds:All G non-syn | % G variants which are non- | #G_ns/#G |
| | % | synonymous | |
| 82 | cds:All MC1 non- | % MC1 variants which are | #MC1_ns/#MC1 |
| | syn % | non-synonymous | |
| 83 | cds:All MC2 non- | % MC2 variants which are | #MC2_ns/#MC2 |
| | syn % | non-synonymous | |
| 84 | cds:All MC3 non- | % MC3 variants which are | #MC3_ns/#MC3 |
| | syn % | non-synonymous | |
| 85 | cds:Other MC2 G | % MC2 Other which are G | #G_MC2_Other/#MC2_Other |
| | % | | |
| 86 | cds:Other G MC2 | % G Other which are at MC2 | #G_MC2_Other/#Other |
| | % | | |
| 87 | cds:Other AT | % A and T Other variants that | (#A_Ti_Other + #T_Ti_Other)/ |
| | Ti/Tv % | are transitions | (#A_Other + #T_Other) |
| 88 | cds:Other C MC2 | % C MC2 Other variants that | #C_MC2_Ti_Other/#C_MC2_Other |
| | Ti/Tv % | are transitions | |
| 89 | cds:Other A MC3 | % A Other which are at MC3 | #A_MC3_Other/#Other |
| | % | | |
| 90 | cds:Other C:G % | % Other variants that are C - | #C_Other/(#C_Other + |
| | | compared to G - strand bias % | #G_Other) |
| 91 | cds:Other C % | number of Other C | #C_Other/#Other |
| | | variants/#Other variants % | |
| 92 | cds:Other T > G % | % Other variants that are | #T > G_Other/#T_Other |
| | | T > G/of OtherT variants | |

In addition to the metrics shown Table E, an additional corresponding set of motif-independent coding region metrics is provided that represent the metrics shown in rows 1-84 of Table E but which are not associated with one of the four primary deaminase motifs (i.e. the AID motif WRC/GYW; the ADAR motif WA/TW, the APOBEC3G motif CC/GG; and the APOBEC3B motif TCW/WGA). Thus, where the metrics in Table D include “all” of the recited metrics in the coding region, including those that fall within one of the four primary deaminase motifs, within one of the secondary deaminase motifs, within a three-mer, or not within any motif, the corresponding “other” metrics include only those metrics shown in rows 1-84 that fall within one of the four primary deaminase motifs. For example, the metric in row 1 of Table E (cds:All A total) is total number of A CDS variants. The corresponding “other” metric” (cds:Other A total) is the total number of CDS A variants that are not associated with (or are not within) one of the four primary deaminase motifs.

2.6.2 Genomic Metrics

Other exemplary metrics include those that are determined across all regions of the genomic nucleic acid sequence are assessed, i.e. regardless of whether the sequence is of a non-coding or coding region. As would be appreciated, these metrics can thus be determined and/or used when the sequence of only a part of the nucleic acid is assessed (e.g. by whole exome sequencing), or whether the sequence of the entire nucleic acid is assessed (e.g. by whole genome sequencing). Exemplary metrics in the genomic metric group include those set forth in Table F. Metrics in rows 11-20 essentially correspond to the metrics in rows 1-10 but which are not associated with one of the four primary deaminase motifs (i.e. the AID motif WKC/GYW; the ADAR motif WA/TW, the APOBEC3G motif CC/GG; and the APOBEC3B motif TCW/WGA). Thus, where the metrics in rows 1-10 of Table F include “all” of the recited metrics in the genomic region, including those that fall within one of the four primary deaminase motifs, within one of the secondary deaminase motifs, within a three-mer or five-mer motif, or not within any motif, the corresponding “other” metrics include only those metrics shown in rows 1-10 that fall within one of the four primary deaminase motifs.

TABLE F
Exemplary genomic metrics

| | Metric Name | Description of metric | Calculation of metric |
|---|---|---|---|
| 1 | g: variant total | Number of all (genomic (g)) | #g (i.e. #SNVs) |
| | (also referred to | variants (i.e. total number of SNVs) | |
| | as “variants in | | |
| | VCF”) | | |
| 2 | g: AT total | # total genomic A and T variants | #g_A + #g_T |
| 3 | g: CG total | # total genomic C and G variants | #g_C + #g_G |
| 4 | g: AT:GC % | % genomic A and T variants | (#g_A + #g_T)/#g |
| 5 | g: A > G + | % A > G and T > C variants of all AT | (#g_A > G + #g_T > C)/ |
| | T > C % | variants | (#g_A + #g_T) |
| 6 | g: A > C + | % A > C and T > G variants of all AT | (#g_A > C + #g_T > G)/ |
| | T > G % | variants | (#g_A + #g_T) |
| 7 | g: A > T + | % A > T and T > A variants of all AT | (#g_A > T + #g_T > A)/ |
| | T > A % | variants | (#g_A + #g_T) |
| 8 | g: C > T + | % C > T and G > A variants of all CG | (#g_C > T + #g_G > A)/ |
| | G > A % | variants | (#g_C + #g_G) |
| 9 | g: C > A + | % C > A and G > T variants of all CG | (#g_C > A + #g_G > T)/ |
| | G > T % | variants | (#g_C + #g_G) |
| 10 | g: C > G + | % C > G and G > C variants of all CG | (#g C > G + #g_G > C)/ |
| | G > C % | variants | (#g_C + #g_G) |
| 11 | g: Other variant | Number of all (genomic) variants | #gO |
| | total | that are not associated with a | |
| | | primary deaminase motif | |
| 12 | g: Other AT total | # total genomic A and T variants | #gO_A + #gO_T |
| | | that are not associated with a | |
| | | primary deaminase motif | |
| 13 | g: Other CG total | # total genomic C and G variants | #gO_C + #gO_G |
| | | that are not associated with a | |
| | | primary deaminase motif | |
| 14 | g: Other AT:GC | % genomic A and T that are not | (#gO_A + #gO_T)/#gO |
| | % | associated with a primary | |
| | | deaminase motif | |
| 15 | g: Other A > G + | % A > G and T > C variants of all AT | (#gO_A > G + #gO_T > C)/ |
| | T > C % | variants that are not associated with | (#gO_A + #gO_T) |
| | | a primary deaminase motif | |
| 16 | g: Other A > C + | % A > C and T > G variants of all AT | (#gO_A > C + #gO_T > G)/ |
| | T > G % | variants that are not associated with | (#gO_A + #gO_T) |
| | | a primary deaminase motif | |
| 17 | g: Other A > T + | % A > T and T > A variants of all AT | (#gO_A > T + #gO_T > A)/ |
| | T > A % | variants that are not associated with | (#gO_A + #gO_T) |
| | | a primary deaminase motif | |
| 18 | g: Other C > T + | % C > T and G > A variants of all CG | (#gO_C > T + #gO_G > A)/ |
| | G > A % | variants that are not associated with | (#gO_C + #gO_G) |
| | | a primary deaminase motif | |
| 19 | g: Other C > A + | % C > A and G > T variants of all CG | (#gO_C > A + #gO_G > T)/ |
| | G > T % | variants that are not associated with | (#gO_C + #gO_G) |
| | | a primary deaminase motif | |
| 20 | g: Other C > G + | % C > G and G > C variants of all CG | (#gO_C > G + #gO_G > C)/ |
| | G > C % | variants that are not associated with | (#gO_C + #gO_G) |
| | | a primary deaminase motif | |
| 21 | g: Motif Hits | Number of “motif” variants in | #g_motif |
| | | genome | |
| 22 | g: Motif % | number of “motif” variants/#g | #g_motif/#g |
| | | variants % | |
| 23 | g: Motif Ti % | number of motif variants which are | #g_motif_Ti/#g |
| | | transitions/#g variants % | |
| 24 | g: Motif C > T + | % motif variants that are C > T or | (#g_motif_C > T + |
| | G > A % | G > A/motif variants | #g_motif_G > A )/#g_motif |
| | g: Motif A > G + | % motif variants that are A > G or | (#g_motif_A > G + |
| | T > C % | T > C/motif variants | #g_motif_T > C )/#g_motif |
| 25 | g: Motif C > A + | % motif variants that are C > A or | (#g_motif_C > A + |
| | G > T % | G > T/motif variants | #g_motif_G > T )/#g_motif |
| | g: Motif A > C + | % motif variants that are A > C or | (#g_motif_A > C + |
| | T > G % | T > G/motif variants | #g_motif_T > G )/#g_motif |
| 26 | g: Motif C > G + | % motif variants that are C > G or | (#g_motif_C > G + |
| | G > C % | G > C/motif variants | #g_motif_G > C )/#g_motif |
| | g: Motif A > T + | % motif variants that are A > T or | (#g_motif_A > T + |
| | T > A % | T > A/motif variants | #g_motif_T > A )/#g_motif |

2.6.3 Assessing a Nucleic Acid Molecule for SNVs Metrics

Any method known in the art for obtaining and assessing the sequence of a nucleic acid molecule can be used in accordance with the methods and systems of the present disclosure. The nucleic acid molecule analyzed using the systems and methods of the present disclosure can be any nucleic acid molecule, although is generally DNA (including cDNA). Typically, the nucleic acid is mammalian nucleic acid, such as human nucleic acid. The nucleic acid can be obtained from any biological sample. For example, the biological sample may comprise a bodily fluid, tissue or cells. In particular examples, the biological sample is a bodily fluid, such as saliva or blood. In some examples, the biological sample is a biopsy. A biological sample comprising tissue or cells may from any part of the body and may comprise any type of cells or tissue.

The nucleic acid molecule can contain a part or all of one gene, or a part or all of two or more genes. Most typically, the nucleic acid molecule comprises the whole genome or whole exome, and it is the sequence of the whole genome or whole exome that is analyzed in the methods of the disclosure. In instances where the whole genome or whole exome is used for analysis, SNVs that are in coding regions or any region (referred to as genome) may be assessed. The examples included herein only analyse the coding region of a gene, also known as the CDS, which is that portion of a gene's DNA or RNA that codes for protein.

When performing the methods of the present disclosure, the sequence of the nucleic acid molecule may have been predetermined. For example, the sequence may be stored in a database or other storage medium, and it is this sequence that is analyzed according to the methods of the disclosure. In other instances, the sequence of the nucleic acid molecule must be first determined prior to employment of the methods of the disclosure. In particular examples, the nucleic acid molecule must also be first isolated from the biological sample.

The biological sample may be any sample suitable for analysis of the nucleic acid of a subject. In particular examples, the biological sample from which the nucleic acid is obtained is a saliva sample or a blood sample.

Methods for obtaining nucleic acid and/or sequencing the nucleic acid are well known in the art, and any such method can be utilized for the methods described herein. In some instances, the methods include amplification of the isolated nucleic acid prior to sequencing, and suitable nucleic acid amplification techniques are well known to a person of ordinary skill in the art. Nucleic acid sequencing techniques are well known in the art and can be applied to single or multiple genes, or whole exomes, transcriptomes or genomes. These techniques include, for example, capillary sequencing methods that rely upon ‘Sanger sequencing’ (Sanger et al. (1977) Proc Natl Acad Sci USA 74: 5463-5467) (i.e., methods that involve chain-termination sequencing), as well as “next generation sequencing” techniques that facilitate the sequencing of thousands to millions of molecules at once. Such methods include, but are not limited to, pyrosequencing, which makes use of luciferase to read out signals as individual nucleotides are added to DNA templates; “sequencing by synthesis” technology (Illumina), which uses reversible dye-terminator techniques that add a single nucleotide to the DNA template in each cycle; and SOLiD™ sequencing (Sequencing by Oligonucleotide Ligation and Detection; Life Technologies), which sequences by preferential ligation of fixed-length oligonucleotides. These next generation sequencing techniques are particularly useful for sequencing whole exomes and genomes. Other exemplary sequencing platforms include third generation (or long-read) sequencing platforms, such as single-molecule nanopore sequencing using the MiniION™ or GridION™ sequencers (developed by Oxford Nanopore and involving passing a DNA molecule through a nanoscale pore structure and then measuring changes in electrical field surrounding the pore), or single molecule real time sequencing (SMRT) utilizing a zero-mode waveguide (ZMW), such as developed by Pacific Biosciences.

Once the sequence of the nucleic acid molecule is obtained, SNVs are then identified. SNVs may be identified by comparing the sequence to a reference sequence. The reference sequence may be the sequence of a nucleic acid molecule from a database, such as reference genome. In particular examples, the reference sequence is a reference genome, such as GRCh38 (hg38), GRCh37 (hg19), NCBI Build 36.1 (hg18), NCBI Build 35 (hg17) and NCBI Build 34 (hg16). In some embodiments, the SNVs are reviewed to remove known single nucleotide polymorphisms (SNPs) from further analysis, such as those identified in the various SNP databases that are publically available. In further embodiments, only those SNVs that are within a coding region of an ENSEMBL gene are selected for further analysis. In addition to identifying the SNVs, the codon containing the SNV and the position of the SNV within the codon (MC-1, MC-2 or MC-3) may be identified. Nucleotides in the flanking 5′ and 3′ codons may also be identified so as to identify the motifs. In some instances of the methods of the present disclosure, the sequence of the non-transcribed strand (equivalent to the cDNA sequence) of the nucleic acid molecules is analyzed. In other instances, the sequence of the transcribed strand is analyzed. In further instances, the sequences of both strands are analyzed.

Having identified one or more SNVs in a nucleic acid molecule, one or metrics can be determined by making the appropriate calculations, as set forth above.

### 3. Kits and Systems for Detecting SNVs and Determining Metrics

All the essential materials and reagents required for detecting SNVs may be assembled together in a kit. For example, when the methods of the present disclosure include first isolating and/or sequencing the nucleic acid to be analyzed, kits comprising reagents to facilitate that isolation and/or sequencing are envisioned. Such reagents can include, for example, primers for amplification of DNA, polymerase, dNTPs (including labelled dNTPs), positive and negative controls, and buffers and solutions. Such kits will also generally comprise, in suitable means, distinct containers for each individual reagent. The kit can also feature various devices, and/or printed instructions for using the kit.

In some embodiments, the methods described generally herein are performed, at least in part, by a processing system, such as a suitably programmed computer system. For example, a processing system can be used to analyze the nucleic acid sequence, identify SNVs, and/or determine metrics. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to identify SNV types, the codon context of an SNV and/or motifs within one or more nucleic acid sequences so as to generate the metrics described herein. In some examples, commands inputted to the processing system by a user assist the processing system in making these determinations. The processing system can also be used to generate a profile or metrics from a sample or subject, and to compare that profile to a reference profile so as to determine a likelihood of a subject having or developing a neurodegenerative disease, as described below.

In one example, a processing system includes at least one microprocessor, a memory, an input/output device, such as a keyboard and/or display, and an external interface, interconnected via a bus. The external interface can be utilised for connecting the processing system to peripheral devices, such as a communications network, database, or storage devices. The microprocessor can execute instructions in the form of applications software stored in the memory to allow the methods of the present disclosure to be performed, as well as to perform any other required processes, such as communicating with the computer systems. The applications software may include one or more software modules, and may be executed in a suitable execution environment, such as an operating system environment, or the like.

### 4. Diagnostic and Therapeutic Applications

Using the methods and systems described herein to detect SNVs in the nucleic acid molecule of a subject, generate one or more metrics, the likelihood that a subject has or will develop a neurodegenerative disease can be determined. Thus, the methods described herein can also be used to facilitate the prescribing of a management program or treatment regimen for a subject. For example, if it is determined that the subject is likely to have or to develop a neurodegenerative disease, then treatment of the subject with an appropriate therapy can be initiated.

As demonstrated in the examples below, subjects who have a neurodegenerative disease have a different profile of metrics compared to those that do not have a neurodegenerative disease. A profile of metrics for a subject, i.e. a sample profile, can therefore be generated and compared to a reference profile of metrics so as to determine whether the subject is likely or unlikely to have or to develop a neurodegenerative disease. Profiles of the present disclosure reflect an evaluation of at least any 1, 2, 3, 4, 5, 6, 7, 8, 9, 10, 20, 30, 40 or more metrics as described above. Reference profiles may correlate with, or be representative of, a healthy phenotype, i.e. a subject that does not have or is unlikely to develop a neurodegenerative disease). When a comparison between the sample profile and the reference profile is made, differences in the profiles can indicate that the subject has or is likely to develop the neurodegenerative disease. In other examples, the reference profile is representative of a subject that has or is likely to develop the neurodegenerative disease. In such examples, a determination that the test subject has or is likely to develop the neurodegenerative disease can be made when the sample profile and the reference profile are essentially the same.

Reference profiles are determined based on data obtained in the evaluation of reference metrics in individuals that have a known phenotype, disease state or risk of developing a disease. Thus, for example, the reference profiles can be based on the data obtained in the evaluation of metrics in individuals that are healthy, i.e. do not have the neurodegenerative disease and/or are unlikely to develop the neurodegenerative disease. In such instances, the reference profile correlates to, or is representative of, a subject that is unlikely to have or to develop the neurodegenerative disease. In other examples, the reference profile is based on the data obtained in the evaluation of metrics in individuals that have or developed a neurodegenerative disease. In such instances, the reference profile correlates to, or is representative of, a subject that is likely to have or to develop the neurodegenerative disease. The individuals used to generate the reference profile may be age, gender and/or ethnicity matched or not.

In some embodiments, reference profiles are generated based on predetermined range intervals or cut-offs for each metric assessed. For example, a reference score is attributed to each metric that is outside a predetermined range interval or is above or below a predetermined cut-off, and the total reference score is then calculated by combining all of the scores. This total reference score is then used to generate a predetermined threshold score, above or below which represents a particular known phenotype, disease state or risk of developing a disease, e.g. below the threshold represents a subject that is unlikely to have or to develop the neurodegenerative disease and above the threshold represents a subject that is likely to have or to develop the neurodegenerative disease. The threshold score therefore represents a score that differentiates those unlikely to have or to develop the neurodegenerative disease from those likely to have or to develop the neurodegenerative disease, and can be readily established by those skilled in the art based on values and scores obtained using control subjects (e.g. positive control subjects known to have have the neurodegenerative disease, and/or negative control subjects known to not have the neurodegenerative disease). The score for each metric may be the same or may be different (e.g. may be “weighted” such that one metric that is outside a predetermined range interval or above or below a cut-off might be given a score that is more or less than another metric). In a particular example, each metric that is outside a predetermined range interval or is above or below a cut-off is given a score of 1.

The predetermined range interval, or cut-off, for a metric can be determined by assessing a metric in two or more subjects that are known to have or be likely to develop the neurodegenerative disease, and/or two or more negative control subjects known to not have or to be unlikely to develop the neurodegenerative disease. In particular examples, the predetermined range interval, or cut-off, is determined by assessing a metric in two or more negative control subjects known to not have or to be unlikely to develop the neurodegenerative disease. A range interval for the metric is then calculated to set the upper and lower limits of what would be considered target values for that metric. A cut-off for the metric can be similarly calculated to set the upper or lower limit of what would be considered target values for that metric. In some examples examples, the range interval is calculated by measuring the average value of the metric plus or minus n standard deviations, whereby the lower limit of the range interval is the average minus n standard deviations and the upper limit of the range interval is the average plus n standard deviations. Cut-off can be similarly calculated. In such examples, n can be 1 or more than or less than 1, e.g. 0.1, 0.2, 0.3, 0.4, 0.5, 0.6, 0.7, 0.8, 0.9, 1, 1.5, 2, 2.5, etc. In still further examples, the upper and lower limits of the predetermined range interval or cut-off are established using receiver operating characteristic (ROC) curves. The subjects used to determine the predetermined range interval or cut-off can be of any age, sex or background, or may be of a particular age, sex, ethnic background or other subpopulation. Thus, in some embodiments, two or more predetermined normal range intervals or cut-offs can be calculated for the same metric, whereby each range interval or cut-off is specific for a particular subpopulation, e.g. a particular sex, age group, ethnic background and/or other subpopulation. The predetermined range interval or cut-off can be determined using any technique know to those skilled in the art, including manual methods of calculation, an algorithm, a neural network, a support vector machine, deep learning, logistic regression with linear models, machine learning, artificial intelligence and/or a Bayesian network.

4.1 Diagnosis of a Neurodegenerative Disease

The methods of the present disclosure can be used to determine the likelihood of a subject having or developing a neurodegenerative disease, such as Mild Cognitive Impairment (MCI), Early Mild Cognitive Impairment (EMCI), Late Mild Cognitive Impairment (LMCI), Alzheimer's disease (AD), Dementia and Parkinson's disease (PD).

In particular embodiments, the likelihood of a subject having or developing MCI or AD is determined by assessing the plurality of metrics set forth in Table 1, or at least 90% of the metrics set forth in Table 1, e.g. at least 90%, 91%, 92%, 93%, 94%, 95%, 96%, 97%, 98% or 99% of the metrics set forth in Table 1. For example, at least 83, 84, 85, 86, 87, 88, 89, 90, 91, 92 or 93 of the metrics set froth in Table 1 can be used to determine the likelihood of a subject having or developing MCI or AD.

In a further embodiment, the likelihood of a subject having or developing EMCI is determined by assessing the plurality of metrics set forth in Table 2, or at least 90% of the metrics set forth in Table 2, e.g. at least 90%, 91%, 92%, 93%, 94%, 95%, 96%, 97%, 98% or 99% of the metrics set forth in Table 2. For example, at least 58, 59, 60, 61, 62, 63 or 64 of the metrics set forth in Table 2 can be used to determine the likelihood of a subject having or developing EMCI.

In another embodiment, the likelihood of a subject having or developing AD is determined by assessing the plurality of metrics set forth in Table 3, or at least 90% of the metrics set forth in Table 3, e.g. at least 90%, 91%, 92%, 93%, 94%, 95%, 96%, 97%, 98% or 99% of the metrics set forth in Table 3. For example, at least 59, 60, 61, 62, 63, 64, 65 or 66 of the metrics set forth in Table 3 can be used to determine the likelihood of a subject having or developing AD.

In still further embodiments, the likelihood of a subject having or developing PD is determined by assessing the plurality of metrics set forth in any one of Tables 4-6, or at least 90% of the metrics set forth in any one of Tables 4-6, e.g. at least 90%, 91%, 92%, 93%, 94%, 95%, 96%, 97%, 98% or 99% of the metrics set forth in Table 4, Table 5 or Table 6. For example, at least 399, 400, 405, 410, 415, 420, 425, 435 or 440 of the metrics set forth in Table 4 can be used to determine the likelihood of a subject having or developing PD; at least 180, 182, 184, 186, 188, 190, 192, 194, 196, 198 or 200 of the metrics set forth in Table 5 can be used to determine the likelihood of a subject having or developing PD; or at least 65, 66, 67, 68, 69, 70 or 71 of the metrics set forth in Table 6 can be used to determine the likelihood of a subject having or developing PD.

4.2 Treatment

The methods of the present invention also extend to therapeutic protocols. In instances where it is determined that a subject is likely to have a neurodegenerative disease, treatment or management protocols may be initiated. Treatment may incude, for example, administration of a therapeutic agent, such as for example, a cognitive enhancer, an anti-inflammatory, an anti-neuropsychiatric. In some examples, further diagnostic tests may be performed to confirm the diagnosis prior to therapy.

In one example, the neurodegenerative disease is Alzheimer's disease, MCI or EMCI, and treatment comprises administration of a cognitive enhancer, an anti-inflammatory, an anti-neuropsychiatric, a cholinesterase inhibitor, an N-methyl-D-aspartate receptor antagonist, an anti-beta amyloid agent (Aβ) agent, and/or an anti-tau agent. In some examples, treatment of Alzheimer's disease, MCI or EMCI comprises administration of any one or more of donepezil, galantamine, rivastigmine, memantine, Aducanumab, levetiracetam, ALZT-OP1, cromolyn+ibuprofen, blarcamesine, AVP-786, AXS-05, Azeliragon, BAN2401, troriluzole, BPDO-1603, Brexpiprazole, CAD106b, COR388, Escitalopram, Gantenerumab, Gantenerumab and solanezumab, Ginkgo biloba, Guanfacine, Icosapent ethyl (IPE), Losartan+amlodipine+atorvastatin, Masitinib, Metformin, Methylphenidate, Mirtazapine, Octohydro-aminoacridine Succinate, Solanezumab, Tricaprilin, TRx0237, or Zolpidem+zoplicone.

In another example, the neurodegenerative disease is Parkinson's disease, and treatment comprises administration of levodopa, a dopamine agonist (e.g. bromocriptine, cabergoline, apomorphine, pramipexole, ropinirole, or rotigotine), a monoamine oxidase-B (MAO B) inhibitor (e.g. selegiline, rasagiline or safinamide), a catechol O-methyltransferase (COMT) inhibitor (e.g. entacapone or tolcapone), an anticholinergic (e.g. enztropine or trihexyphenidyl), amantadine, an adenosine A_{2A} antagonist (e.g. istradefylline), Cu-ATSM, a cell therapy (e.g. mesenchymal stem cells, or neural stem cells), a kinase inhibitor (e.g. DNL 151, FB-101, saracatinib), a neurotropic factor (e.g. GDNF or CDNF), or a GLP-1 agonist (e.g. exenatide).

In some instances, where a metric is indicative of the activity of a deaminase, therapy or preventative measures may include administration to the subject of an inhibitor of that deaminase. Inhibitors can include, for example, siRNAs, miRNAs, protein antagonists (e.g., dominant negative mutants of the mutagenic agent), small molecule inhibitors, antibodies and fragments thereof. For example, commercially available siRNAs and antibodies specific for APOBEC cytidine deaminases and AID are widely available and known to those skilled in the art. Other examples of APOBEC3G inhibitors include the small molecules described by Li et al. (ACS. Chem. Biol., (2012) 7(3): 506-517), many of which contain catechol moieties, which are known to be sulfhydryl reactive following oxidation to the orthoquinone. APOBEC1 inhibitors also include, but are not limited to, dominant negative mutant APOBEC1 polypeptides, such as the mul (H61K/C93S/C96S) mutant (Oka et al., (1997) J. Biol. Chem. 272: 1456-1460).

Typically, therapeutic agents will be administered in pharmaceutical compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of the neurodegenerative disease. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner, and those of skill in the art may readily determine suitable dosages of the therapeutic agents and suitable treatment regimens without undue experimentation.

In order that the invention may be readily understood and put into practical effect, particular preferred embodiments will now be described by way of the following non-limiting examples.

### EXAMPLES

### Example 1

### Methods for Determining Metrics

Whole genome sequences from subjects were analyzed to identify single nucleotide variants (SNVs). Briefly, sequences were formatted in a .vcf file using the hg37 genome coordinates as a reference.

Each variant in the .vcf file was analyzed and selected for further consideration if it was a simple single nucleotide substitution and was not an insertion or deletion. The following steps were then performed:
a) the codon context within the structure of the affected codon (MC) was determined, i.e. the position of the SNV within the encoding triplet was determined, wherein the first position (read from 5′ to 3′) is referred to as MC1 (or MC-1 site), the second position is referred to as MC2 (or MC-2 site) and the third position is referred to as MC3 (or MC-3 site);
b) a nine-base window was extracted from the surrounding genome sequence such that the sequence of three complete codons was obtained. The direction of the gene was used for determining 5′ and 3′ directions, and for determining the correct strand of the nine bases. The nine-base window was always reported according to the direction of the gene such that bases in the window around variants in genes on the reverse strand of the genome are reverse complimented in relation to the genome, but in the forward direction in relation to the gene. By convention, this context is always reported in the same strand of the gene. Positive strand genes will have codon context bases from the positive strand of the reference genome, and negative strand genes will have codon context bases from the negative strand of the reference genome;
c) motif searching was performed using motifs described in Table B and C to determine whether the variation was within such a motif.

Metrics set forth in Tables D-F were then calculated.

### Example 2

### Metrics for Differentiating Subjects with Cognitive Impairment

Various combinations of metrics were used to assess patients with cognitive impairment.

Sequence data was supplied by the Alzheimer's Disease Neuroimaging Initiative (ADNI). ADNI is a global research project that actively supports studies that can slow or stop the progression of AD. In this multi-site longitudinal study, researchers at 63 sites in the US and Canada tracked the progression of AD in the human brain with clinical, imaging, genetic and biospecimen biomarkers through the process of normal aging, early mild cognitive impairment (EMCI), and late mild cognitive impairment (LMCI) to dementia or AD. Due to racial differences, some examples present data for all individuals, and other examples present data for “white” individuals only.

Based on clinical, cognitive assessment, radiological and molecular pathology results, the samples analyzed were categorized into the following groups:
MCI—Mild Cognitive Impairment (n=363 “white”; n=24 “non-white”)
EMCI—Early Mild Cognitive Impairment (n=29 “white”; n=4 “non-white”)
LMCI—Late Mild Cognitive Impairment (n=21 “white”; n=1 “non-white”)
Alzheimer's disease (AD) (n=31 “white”; n=0 “non-white”)
Dementia (n=52 “white”; n=2 “non-white”)
CN—Control Normals (n=260 “white”; n=21 “non-white”)
   Staging of MCI (early or late) was determined using the Wechsler Memory Scale Logical Memory II.
   Comparison of Diseased Subjects with Control Subjects

All subjects were included in this example, regardless of race. Metrics used to differentiate patients with cognitive impairment from control (i.e. non-diseased) subjects (CN) are shown in Table 1. The average value for each metric in the genome of each control subject, and the standard deviation, was calculated. The range interval (RI), which is the average ±one standard deviation, for each metric was determined from the CN subject group.

Metrics were then calculated for all CN, MCI, LMCI, Dementia and AD subjects. Whether the value for each metric was higher (HIGH) or lower (LOW) than the RI (i.e. whether it was lower than the average of the CN subjects minus one standard deviation or whether it was higher than the average of the CN subjects plus one standard deviation) was then determined. The total number of metrics that were higher than the RI and the total number of metrics that were lower than the RI were used to calculate a CI score. The CI score was calculated as HIGHs minus LOWs plus a constant (i.e. patient CI score is the number of metrics with values higher than the RI minus the number of metrics with values lower than the RI plus 50; the constant is added to make all scores non-negative).

Table 1, below, shows the results of this assessment, and demonstrates that the profile of representative subjects with cognitive impairment and AD is different to control (CN) subjects.

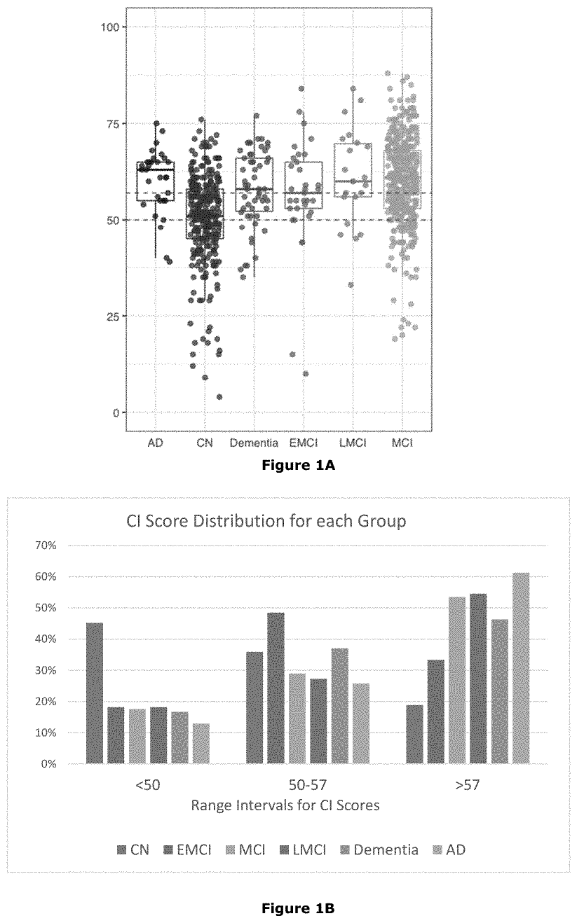

CI scores calculated using the metrics shown in Table 1 for each individual with MCI, EMCI, LMCI, AD, dementia, as well as each CN subject, are shown in FIG. 1A. Statistics including Sensitivity and Specificity of the test using a cognitive impairment score of <50 or >57 are as follows:

| | With Disease | Disease not Present |
|---|---|---|
| Positive | 115 | 84 |
| Negative | 74 | 311 |
| Total | 189 | 395 |
| | Sensitivity= | 61% |
| | Specificity= | 79% |

The bar graph shown in FIG. 1B shows the relative proportions (as %) of subjects from each cohort that have a CI score that falls below 50, is within the range 50-57, or is above 57.

Comparison of EMCI Subjects with Control Subjects

Metrics shown in Table 2 were calculated from the genome sequences of control (i.e. non-diseased) subjects (CN). All “non-white” subjects were excluded from this example. The average value for each metric in the genome of control (CN) subjects, and the standard deviation, was then calculated and a cut-off was determined. The cut-off was calculated to be greater than the average or the average plus 0.5×, 1× or 2× the standard deviation; or less than the average or the average minus 0.5×, 1× or 2× the standard deviation, as shown in Table 2. As can be be seen from Table 2, some metrics were used to determine more that one cut-off, i.e. a cut-off below a first value for that metric and and a cutoff above a second value for that matric (see e.g. the metric of “variants in VCF” where there is a cut-off of >3502542 and a cutoff of <3382123).

The values for the chosen metrics were then calculated for control (CN) subjects and EMCI subjects. Representative profiles and CI scores are presented for two control subject and three subjects with EMCI. The values of each of these metrics was compared to the relevant cut-off to determine whether they were above or below the cut-off. If they were outside the cut-off, they were assigned a score of 1. The total number of metrics that were higher than the cutoff and the total number of metrics that were lower than the cutoff were added to create a total, or an EMCI score. The EMCI score is shown at the bottom of Table 2 for each subject.

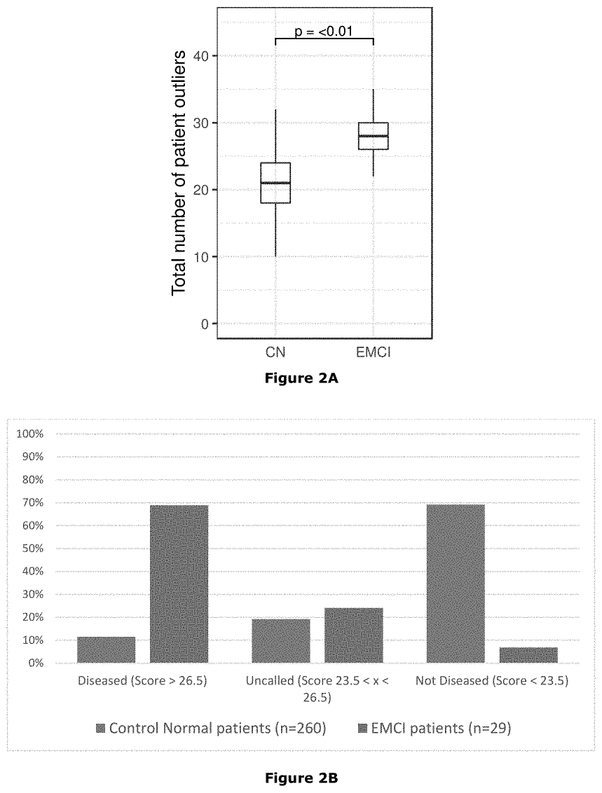

As can be seen from Table 2, the profiles of CN and EMCI subjects generated using the metrics set forth in Table 2 are different. This is also shown in FIG. 2, where EMCI scores for each of the CN and EMCI subjects in the study cohort are provided in a box plot. This analysis suggests that an EMCI score could be used to differentiate between subjects that are unlikely to have EMCI and subjects that are likely to have EMCI. The sensitivity and specificity of the EMCI score using <23.5 or >26.5 as a cut-off is as follows:

| | With Disease | Disease not Present |
|---|---|---|
| Score >26.5 | 20 | 30 |
| Score 23.5 < x < 26.5 | 7 | 50 |
| Score >23.5 | 2 | 180 |
| Total | 29 | 260 |

| | |
|---|---|
| Sensitivity= | 91% |
| Specificity= | 86% |
| Positive Predictive Value (PPV)= | 40% |
| Negative Predictive Value (NPV)= | 99% |

The bar graph shown in FIG. 2B shows the relative proportions (as %) of subjects from the Controls cohort and the EMCI cohort that fall below 23.5, within the range 23.5-26.5 (i.e. 23.5<x<26.5), or above 26.5.

Comparison of AD Subjects with Control Subjects

Metrics shown in Table 3 were derived from the genome sequences of control (CN, white only) subjects. The average value for each metric in the genome of each control (CN) subject, and the standard deviation, was then calculated and a cut-off was determined. The cut-off was calculated to be greater than the average or the average plus n x the standard deviation; or less than the average or the average minus n x standard deviation, as shown in Table 3.

The values for the chosen metrics were then calculated for control (CN) subjects and AD subjects. Representative data is presented for two control (CN_84 and CN_72) subjects and two subjects with AD (AD_78 and AD_73). The values of each of these metrics was compared to the relevant cut-off to determine whether they were above or below the cut-off (i.e. within or outside the range interval). The number of outliers per subject was added to produce an AD score. This is shown at the bottom of Table 3 for each representative subject.

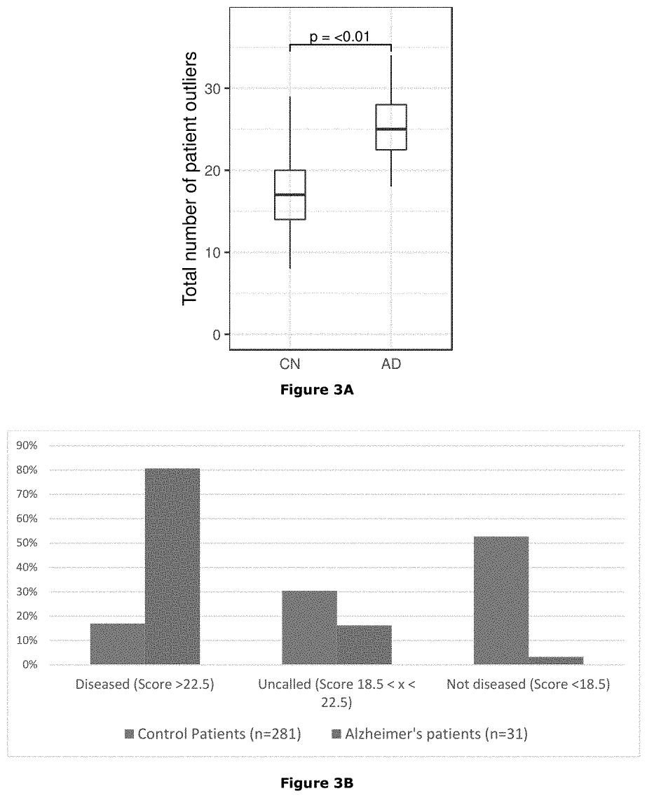

As can be seen from Table 3, the profiles of CN and AD subjects generated using the metrics set forth in Table 3 are different. This is also shown in FIG. 3, where AD scores for each of the CN and AD patients in the study cohort are plotted as an average with standard deviation. Further analysis suggests that an AD score could be used to differentiate between subjects that are unlikely to have AD and subjects that are likely to have AD. The sensitivity and specificity of the AD score using >22.5 or <18.5 as a cut-off is as follows:

| | With Disease | Disease not Present |
|---|---|---|
| Score >22.5 | 25 | 44 |
| Score 18.5 < x < 22.5 | 6 | 130 |
| Score <18.5 | 0 | 86 |
| Total | 31 | 260 |

| | |
|---|---|
| Sensitivity= | 100% |
| Specificity= | 66% |
| Positive Predictive Value (PPV)= | 36% |
| Negative Predictive Value (NPV)= | 100% |

The bar graph shown in FIG. 3C shows the relative proportions (as %) of subjects from each cohort that fall below 18.5, within the range 18.5-22.5, or above 22.5.

### Example 3

### Metrics for Differentiating Subjects with Parkinson's Disease

Data for this study was obtained from the whole genomes of subjects participating in the Parkinson's Progression Markers Initiative (PPMI) funded by The Michael J. Fox Foundation for Parkinson's Research Foundation (MJFF).

Whole genomes for the following groups of subjects were included in this analysis:
Control Normals (CN) (n=196)—Control subjects without PD who are 30 years or older and who do not have a first-degree blood relative with PD.
Parkinson's disease (PD) (n=479)—Subjects with a diagnosis of PD for two years or less who are not taking PD medications.

Of these subjects, a subset consisting of the whole genomes of the first 150 CN subjects, and the first 350 PD subjects were used to develop and evaluate a PD test. The whole genomes of the remaining subjects were used to validate the initial test design.

The initial PD test design was conducted using cut-offs to identify outliers for 3 different sets of metrics:
SET A—A large set of 443 metrics that include many types of measures associated with SNVs for codon-contexted SNVs of A, G, C and T (see Table 4).
SET B—A subset of SET A consisting of 201 metrics from SET A that includes only those deaminase metrics associated with A-to-I editing events and known to play a key role in regulating CNS function (see Table 5).
SET C—A limited subset of SET A consisting of 72 mixed metrics, selected by choosing those metrics for which there was found to be >40% difference between the average score per CN subject metric and AD subject metrics (SD multiplier 1.0 for all metrics) (see Table 6).

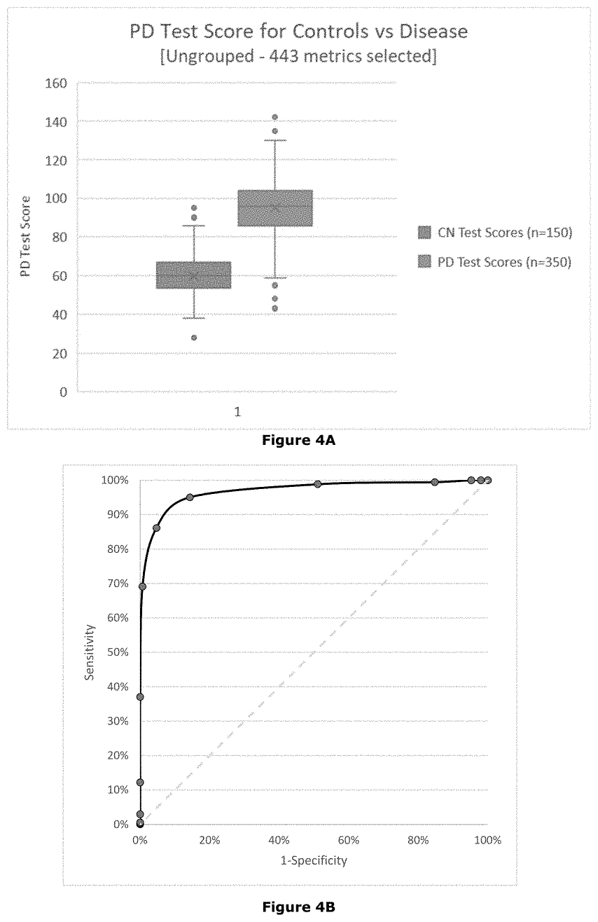

As shown in FIGS. 4-6, each of the sets of metrics could be used to develop profiles and tests that could distinguish between subject that are unlikely to have PD and subjects that are likely to have PD.

FIG. 4 shows the analysis of the differentiation of CN and PD subjects on the basis of the metrics shown in Table 4. A PD score was given to each subject on the basis of this, with FIG. 4A showing a box plot of PD scores. The sensitivity and specificity using various PD threshold (or cut-off) scores is shown in FIG. 4B as an ROC curve and is as follows:

| | | | | | | | | | | | | | | |
|---|---|---|---|---|---|---|---|---|---|---|---|---|---|---|
| Sensitivity | 0% | 0.3% | 0.6% | 3.1% | 12.0% | 34.9% | 66.9% | 85.1% | 94.9% | 98.3% | 99.4% | 100.0% | 100.0% | 100.0% |
| Specificity | 100% | 100% | 100% | 100.0% | 100.0% | 100.0% | 99.3% | 95.3% | 86.0% | 51.3% | 18.7% | 7.3% | 2.7% | 0% |
| Test Cutoff Score | 150 | 140 | 130 | 120 | 110 | 100 | 90 | 80 | 70 | 60 | 50 | 40 | 30 | 20 |

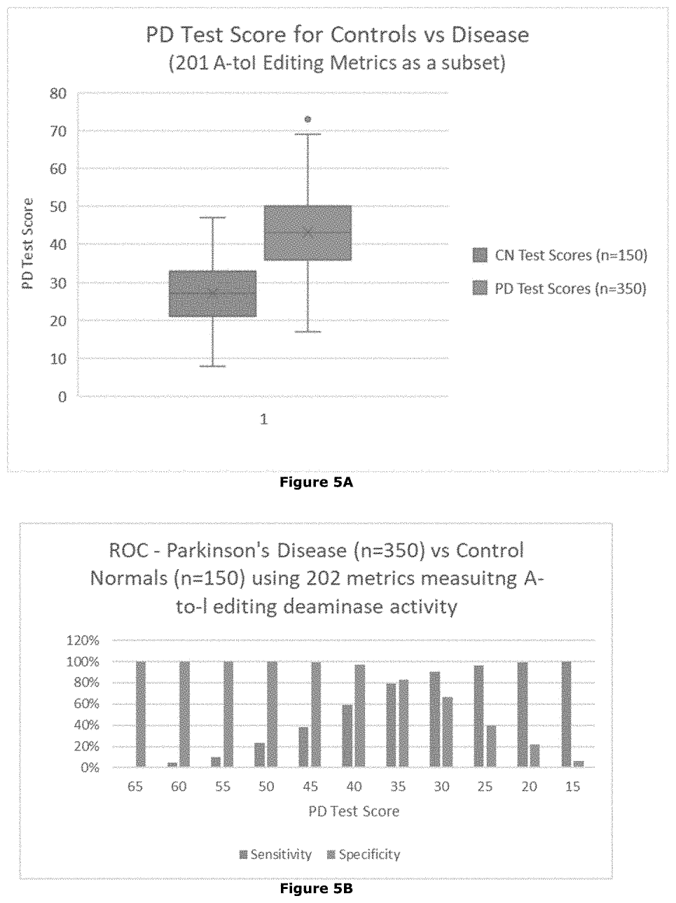

FIG. 5 shows the analysis of the differentiation of CN and PD subjects on the basis of the metrics shown in Table 5. A PD score was given to each subject on the basis of this, with FIG. 5A showing a box plot of PD scores. The sensitivity and specificity using various PD threshold (or cut-off) scores is shown in FIG. 4B as an ROC curve and is as follows:

| | | | | | | | | | | | |
|---|---|---|---|---|---|---|---|---|---|---|---|
| Sensitivity | 1% | 5.1% | 9.7% | 23.1% | 38.6% | 59.4% | 79.1% | 90.6% | 96.0% | 99.1% | 100.0% |
| Specificity | 100% | 100% | 100% | 100.0% | 99.3% | 96.7% | 82.7% | 66.7% | 40.0% | 22.0% | 6.0% |
| Test Cutoff Score | 65 | 60 | 55 | 50 | 45 | 40 | 35 | 30 | 25 | 20 | 15 |

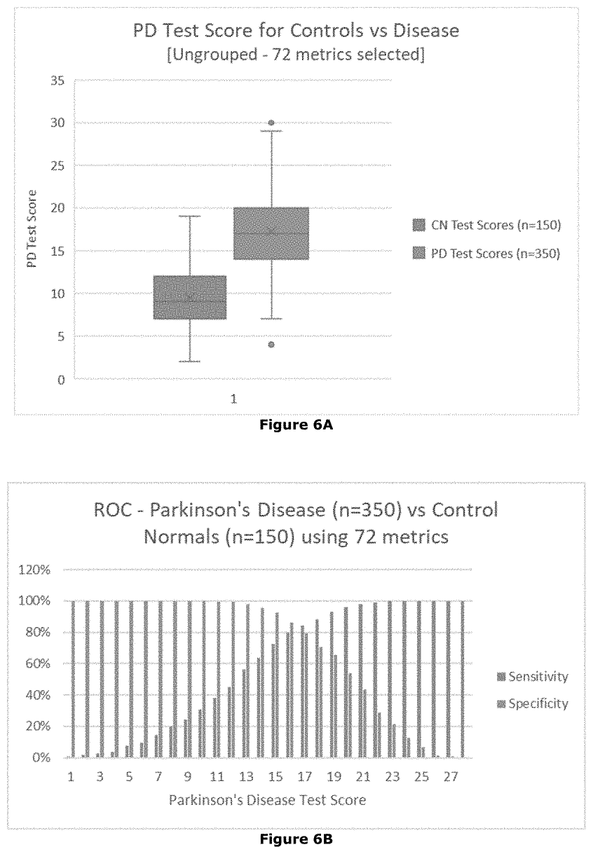

FIG. 6 shows the analysis of the differentiation of CN and PD subjects on the basis of the metrics shown in Table 6. A PD score was given to each subject on the basis of this, with FIG. 6A showing a box plot of PD scores. The sensitivity and specificity using various PD threshold (or cut-off) scores is shown in FIG. 6B and as follows:

| | | | | | | | | | | | | | | |
|---|---|---|---|---|---|---|---|---|---|---|---|---|---|---|
| Sensitivity (%) | 1 | 2 | 3 | 4 | 7 | 9 | 14 | 20 | 24 | 31 | 38 | 45 | 56 | 64 |
| Specificity (%) | 100 | 100 | 100 | 100 | 100 | 100 | 100 | 100 | 100 | 100 | 99 | 99 | 98 | 95 |
| Test cutoff score | 28 | 27 | 26 | 25 | 24 | 23 | 22 | 21 | 20 | 19 | 18 | 17 | 16 | 15 |

| | | | | | | | | | | | | | | |
|---|---|---|---|---|---|---|---|---|---|---|---|---|---|---|
| Sensitivity (%) | 73 | 80 | 84.3 | 88.3 | 92.9 | 95.7 | 97.7 | 99.1 | 99.7 | 99.7 | 99.7 | 100 | 100 | 100 |
| Specificity (%) | 93 | 86 | 79 | 70.7 | 65.3 | 54 | 43.3 | 28.7 | 21.3 | 12.7 | 6.7 | 1.3 | 0.7 | 0 |
| Test cutoff score | 14 | 13 | 12 | 11 | 10 | 9 | 8 | 7 | 6 | 5 | 4 | 3 | 2 | 1 |

TABLE 1

| | | | | | | Example profiles and CI Scores for representative subjects; | | | | | |
|---|---|---|---|---|---|---|---|---|---|---|---|
| | | | | | | “HIGH” = higher than the RI, “LOW” = lower than the RI | | | | | |
| | | CN | CN | Average − | Average + | 003_S_4555 | 023_S_4241 | 002_S_1268 | 072_S_4057 | 094_S_4162 | 003_S_4136 |
| Metric name | Motif | Average | SD | 1SD | 1SD | CN | EMCI | MCI | LMCI | Dementia | AD |
| cds: A3G MC2 % | C-C- | 19.424 | 0.462 | 18.962 | 19.885 | LOW | | | | | |
| cds: A3G C > T at MC2 % | C-C- | 17.425 | 0.649 | 16.776 | 18.074 | | | | HIGH | HIGH | HIGH |
| cds: A3G non-syn % | C-C- | 41.572 | 0.619 | 40.953 | 42.192 | LOW | | | | | |
| cds: A3G C > T at MC2 motif % | C-C- | 6.294 | 0.256 | 6.038 | 6.550 | LOW | | | HIGH | HIGH | HIGH |
| cds: A3G C > G at MC1 motif % | C-C- | 2.638 | 0.173 | 2.464 | 2.811 | HIGH | | HIGH | HIGH | LOW | LOW |
| cds: A3G C > T at MC2 cds % | C-C- | 1.075 | 0.044 | 1.031 | 1.119 | LOW | | | HIGH | HIGH | HIGH |
| cds: A3G C > G at MC1 cds % | C-C- | 0.451 | 0.030 | 0.421 | 0.480 | HIGH | | HIGH | HIGH | LOW | LOW |
| cds: Gen2_CCT C > G at MC1 cds % | C-C-T | 0.137 | 0.015 | 0.121 | 0.152 | | LOW | | HIGH | | LOW |
| cds: Gen2_GCC G > C at MC2 % | G-C-C | 46.360 | 3.609 | 42.751 | 49.969 | | HIGH | HIGH | | | |
| cds: Gen2_GCC G > C at MC2 motif % | G-C-C | 5.524 | 0.558 | 4.966 | 6.081 | | | | HIGH | HIGH | |
| g: Gen2_TCG C > A + G > T g % | T-C-G | 0.197 | 0.001 | 0.196 | 0.199 | | | HIGH | | HIGH | |
| cds: Gen2_CCG C > G at MC1 motif % | C-C-G | 1.095 | 0.142 | 0.952 | 1.237 | | HIGH | | HIGH | | LOW |
| cds: Gen2_CCG C > G at MC1 cds % | C-C-G | 0.092 | 0.012 | 0.080 | 0.105 | | HIGH | | HIGH | | LOW |
| cds: ADAR_Gen2_AAA Ti A:T % | A-A-A | 63.497 | 1.608 | 61.889 | 65.105 | | | LOW | LOW | | HIGH |
| cds: ADAR_Gen2_TAA T > G at MC3 % | T-A-A | 67.266 | 6.318 | 60.948 | 73.583 | | HIGH | HIGH | | | HIGH |
| cds: ADAR_Gen2_AAC A > G at MC1 % | A-A-C | 21.093 | 1.679 | 19.415 | 22.772 | HIGH | | | | | HIGH |
| cds: ADAR_Gen2_AAC A > G at MC1 motif % | A-A-C | 9.694 | 0.801 | 8.894 | 10.495 | HIGH | | | LOW | | |
| cds: ADAR_Gen2_AAC A > G at MC1 cds % | A-A-C | 0.221 | 0.019 | 0.202 | 0.240 | HIGH | | | LOW | | |
| cds: ADAR_Gen2_GAG T > C % | G-A-G | 65.023 | 1.436 | 63.587 | 66.459 | | | | | HIGH | HIGH |
| cds: ADAR_Gen2_GAG T Ti/Tv % | G-A-G | 65.023 | 1.436 | 63.587 | 66.459 | | | | | HIGH | HIGH |
| cds: ADAR_Gen2_GAG A non-syn % | G-A-G | 54.331 | 1.760 | 52.571 | 56.091 | | | | | | |
| cds: ADAR_Gen2_GAG T > C cds % | G-A-G | 0.850 | 0.031 | 0.819 | 0.882 | | | HIGH | | | |
| cds: AIDd G > C at MC2 % | WR-C-Y | 40.259 | 2.615 | 37.644 | 42.873 | LOW | | HIGH | | HIGH | |
| cds: ADARb A > G at MC1 % | W-A-Y | 29.025 | 0.947 | 28.078 | 29.971 | | | | HIGH | | HIGH |
| cds: ADARb A > G at MC1 motif % | W-A-Y | 13.303 | 0.449 | 12.854 | 13.752 | | | LOW | HIGH | | HIGH |
| cds: ADARg T > A at MC3 % | W-A-A | 32.219 | 5.947 | 26.271 | 38.166 | | | HIGH | | | LOW |
| g: A3Gb C > A + G > T g % | -C-G | 1.219 | 0.005 | 1.214 | 1.224 | | | HIGH | | | |
| g: A3Gb C > A + G > T % | -C-G | 7.946 | 0.042 | 7.904 | 7.988 | | | | | | |
| g: A3Ge C > A + G > T g % | SC-C-GS | 0.095 | 0.001 | 0.094 | 0.096 | | | | HIGH | | |
| g: A3Ge C > A + G > T % | SC-C-GS | 7.620 | 0.088 | 7.533 | 7.708 | | | | | | |
| cds: A3Gf non-syn % | SC-C-G | 42.356 | 1.098 | 41.258 | 43.454 | LOW | HIGH | | | | LOW |
| g: A3Gf C > A + G > T % | SC-C-G | 8.396 | 0.075 | 8.321 | 8.471 | | | | | | |
| cds: A3Gg C > G at MC1 % | C-C-GS | 24.274 | 3.111 | 21.164 | 27.385 | LOW | HIGH | | | | |
| cds: A3Gg C > G at MC1 motif % | C-C-GS | 1.435 | 0.208 | 1.227 | 1.643 | | HIGH | | | | |
| cds: A3Gg C > G at MC1 cds % | C-C-GS | 0.069 | 0.010 | 0.059 | 0.079 | | HIGH | | | | |
| g: A3Gg C > A + G > T % | C-C-GS | 7.638 | 0.073 | 7.564 | 7.711 | | | | | | |
| cds: A3Gh C > G at MC1 motif % | S-C-GS | 1.132 | 0.149 | 0.982 | 1.281 | | HIGH | | | | |
| cds: A3Gh C > G at MC1 cds % | S-C-GS | 0.095 | 0.013 | 0.083 | 0.108 | | HIGH | | | | |
| g: A3Gh C > G + G > C % | S-C-GS | 7.583 | 0.057 | 7.525 | 7.640 | | | | | | |
| cds: A3Gi G > C at MC2 motif % | SG-C-G | 0.784 | 0.215 | 0.569 | 0.998 | HIGH | HIGH | HIGH | | HIGH | |
| cds: A3Gi C > G at MC1 cds % | SG-C-G | 0.011 | 0.005 | 0.006 | 0.015 | | HIGH | HIGH | HIGH | HIGH | HIGH |
| cds: A3Gi G > C at MC2 cds % | SG-C-G | 0.021 | 0.006 | 0.015 | 0.027 | HIGH | HIGH | HIGH | HIGH | HIGH | HIGH |
| g: A3Gi C > G + G > C % | SG-C-G | 7.618 | 0.084 | 7.534 | 7.702 | LOW | | | | | HIGH |
| cds: A3Be G > A at MC1 % | YT-C-A | 32.012 | 4.115 | 27.898 | 36.127 | | | HIGH | | | |
| cds: A3Be G > A at MC1 motif % | YT-C-A | 8.349 | 1.184 | 7.165 | 9.534 | | | | | | |
| cds: A3Be G > A at MC1 cds % | YT-C-A | 0.087 | 0.013 | 0.074 | 0.101 | | | | | | |
| cds: A1 C > A at MC2 % | -C-A | 21.098 | 1.813 | 19.285 | 22.910 | | | | | | |
| cds: A1 C > A at MC2 motif % | -C-A | 1.899 | 0.193 | 1.706 | 2.092 | | | | | | |
| g: ADAR_Gen1_ATC % | -A-TC | 2.861 | 0.006 | 2.855 | 2.867 | LOW | | LOW | HIGH | | HIGH |
| g: ADAR_Gen1_ATC A > G + T > C g % | -A-TC | 2.068 | 0.005 | 2.063 | 2.073 | LOW | | LOW | | | HIGH |
| cds: ADAR_Gen1_ACC A > G at MC1 % | -A-CC | 36.664 | 1.457 | 35.207 | 38.120 | | | | | | HIGH |
| cds: ADAR_Gen1_ACC A > G at MC1 motif % | -A-CC | 14.831 | 0.637 | 14.194 | 15.468 | | | | | | HIGH |
| cds: ADAR_Gen1_ACC A > G at MC1 cds % | -A-CC | 0.494 | 0.023 | 0.471 | 0.517 | | HIGH | | | | HIGH |
| cds: ADAR_Gen1_AGTA > T % | -A-GT | 12.336 | 1.134 | 11.202 | 13.470 | | HIGH | | | | |
| cds: ADAR_Gen1_AGG Ti % | -A-GG | 2.755 | 0.056 | 2.700 | 2.811 | | LOW | HIGH | | HIGH | |
| g: ADAR_Gen1_AGG % | -A-GG | 2.787 | 0.007 | 2.779 | 2.794 | LOW | | | | | HIGH |
| cds: ADAR_Gen3_AAA MC3 % | AA-A- | 53.136 | 1.365 | 51.772 | 54.501 | HIGH | HIGH | | | LOW | |
| cds: ADAR_Gen3_CAA A > G at MC1 % | CA-A- | 20.242 | 1.179 | 19.063 | 21.422 | | | | | | HIGH |
| cds: ADAR_Gen3_CAA A > G at MC1 motif % | CA-A- | 9.552 | 0.592 | 8.959 | 10.144 | | | | | | HIGH |
| g: ADAR_Gen3_GGA A > G + T > C g % | GG-A- | 1.473 | 0.005 | 1.468 | 1.478 | | LOW | HIGH | | HIGH | |
| cds: Gen1_CAA MC2 % | -C-AA | 16.274 | 1.098 | 15.176 | 17.372 | | | | | | HIGH |
| cds: Gen1_CTA MC2 % | -C-TA | 31.435 | 1.890 | 29.544 | 33.325 | HIGH | | HIGH | LOW | | |
| cds: Gen1_CTA G > C at MC2 motif % | -C-TA | 4.618 | 0.880 | 3.737 | 5.498 | | | HIGH | HIGH | LOW | HIGH |
| cds: Gen1_CAT C > A at MC2 % | -C-AT | 20.246 | 3.477 | 16.769 | 23.723 | HIGH | | HIGH | HIGH | | |
| g: Gen1_CTT C > T + G > A g % | -C-TT | 2.457 | 0.007 | 2.451 | 2.464 | | | | | | HIGH |
| cds: Gen1_CGC C > G at MC1 % | -C-GC | 24.498 | 3.749 | 20.749 | 28.246 | | HIGH | | | LOW | HIGH |
| cds: Gen1_CGC C > G at MC1 motif % | -C-GC | 0.918 | 0.156 | 0.762 | 1.074 | | | | | LOW | HIGH |
| cds: Gen1_CGC C > G at MC1 cds % | -C-GC | 0.056 | 0.010 | 0.046 | 0.066 | | | | | LOW | HIGH |
| cds: Gen1_CCG G > T at MC1 % | -C-CG | 20.006 | 5.767 | 14.239 | 25.773 | LOW | | HIGH | HIGH | | HIGH |
| cds: Gen1_CGG G > C motif % | -C-GG | 5.497 | 0.299 | 5.198 | 5.797 | | | LOW | HIGH | HIGH | |
| cds: Gen1_CGG G > C cds % | -C-GG | 0.436 | 0.024 | 0.412 | 0.460 | | | LOW | HIGH | HIGH | |
| g: Gen1_CGG C > A + G > T g % | -C-GG | 0.311 | 0.002 | 0.309 | 0.313 | | | | | HIGH | |
| g: Gen1_CGG C > A + G > T % | -C-GG | 6.989 | 0.046 | 6.943 | 7.036 | | | | | | HIGH |
| g: Gen1_CGG C > G + G > C g % | -C-GG | 0.407 | 0.003 | 0.405 | 0.410 | | | | | | |
| g: Gen1_CGG C > G + G > C % | -C-GG | 9.167 | 0.070 | 9.097 | 9.238 | | | | | | |
| cds: Gen3_TCC C > G % | TC-C- | 15.074 | 1.117 | 13.958 | 16.191 | | | | | | HIGH |
| cds: Gen3_TCC C > G motif % | TC-C- | 6.444 | 0.520 | 5.924 | 6.964 | | LOW | | | | HIGH |
| g: Gen3_TCC C > A + G > T % | TC-C- | 14.988 | 0.085 | 14.904 | 15.073 | | | HIGH | | | HIGH |
| cds: Gen3_TGC C > T at MC3 % | TG-C- | 46.338 | 2.224 | 44.113 | 48.562 | | HIGH | | | HIGH | |
| cds: Gen3_CCC C > G at MC1 % | CC-C- | 29.692 | 2.613 | 27.079 | 32.305 | | | HIGH | | | LOW |
| cds: Gen3_CCC C > G at MC1 cds % | CC-C- | 0.178 | 0.019 | 0.159 | 0.197 | HIGH | HIGH | HIGH | HIGH | | LOW |
| cds: Gen3_CGC G > C % | CG-C- | 16.745 | 1.667 | 15.078 | 18.412 | | | | | HIGH | |
| cds: Gen3_CGC C > G at MC1 % | CG-C- | 24.710 | 5.934 | 18.776 | 30.644 | | | | HIGH | HIGH | |
| cds: Gen3_CGC G > C at MC2 % | CG-C- | 24.305 | 4.368 | 19.937 | 28.673 | | | | | HIGH | |
| cds: Gen3_CGC G > C motif % | CG-C- | 8.584 | 0.882 | 7.702 | 9.467 | | | | | | |
| cds: Gen3_CGC G > C at MC2 motif % | CG-C- | 2.089 | 0.438 | 1.651 | 2.526 | | | | HIGH | HIGH | |
| cds: Gen3_CGC G > C at MC2 cds % | CG-C- | 0.038 | 0.008 | 0.030 | 0.045 | | | | HIGH | HIGH | |
| cds: Gen3_GAC G > C at MC2 motif % | GA-C- | 1.235 | 0.175 | 1.060 | 1.409 | LOW | LOW | | | | |
| cds: Gen3_GAC G > C at MC2 cds % | GA-C- | 0.052 | 0.007 | 0.045 | 0.059 | LOW | LOW | | | | |
| g: Gen3_GGC C > G + G > C % | GG-C- | 14.449 | 0.081 | 14.368 | 14.530 | LOW | HIGH | | | | |

| | | | | | | |
|---|---|---|---|---|---|---|
| HIGHs | 11 | 20 | 22 | 24 | 22 | 32 |
| LOWS | 15 | 6 | 6 | 4 | 7 | 9 |
| CI Score | 46 | 64 | 66 | 70 | 65 | 73 |

TABLE 2

| | | Mean | Mean | | |
|---|---|---|---|---|---|
| Metric | Motif | CN | EMCI | Cutoff | 0610_CN |
| cds: AID Hits | WR-C- | 3080.29 | 3070.97 | <3059.851364 | 3047 |
| cds: Gen2_TCA C > A at MC2 % | T-C-A | 3.11 | 2.72 | <0.609375824 | 2.857 |
| cds: Gen2_TCT G > T at MC1 % | T-C-T | 23.80 | 23.01 | <23.37145999 | 22.727 |
| cds: Gen2_TCT G > T at MC1 motif % | T-C-T | 1.15 | 1.09 | <1.137150176 | 0.971 |
| cds: Gen2_TCC G > T at MC2 % | T-C-C | 24.47 | 22.13 | <24.71428 | 26.316 |
| cds: Gen2_TCG G > T at MC2 % | T-C-G | 15.51 | 13.58 | <14.93731994 | 25 |
| cds: ADAR_Gen2_TAA T > G at MC1 % | T-A-A | 27.15 | 26.39 | <27.33995 | 35.714 |
| cds: AIDe G > T at MC2 motif % | WR-C-GW | 0.37 | 0.29 | <0.153920871 | 0.524 |
| cds: ADARe A > C at MC1 % | CW-A-A | 16.66 | 15.09 | <10.13023448 | 26.316 |
| cds: ADARj T > G at MC2 % | S-A-RA | 9.93 | 9.62 | <8.534817717 | 9.434 |
| cds: A3Gd G > C at MC2 motif % | SC-C-GW | 0.54 | 0.50 | <0.438505325 | 0.679 |
| cds: A3Ge C > A at MC2 % | SC-C-GS | 13.85 | 13.48 | <13.703125 | 14.286 |
| cds: A3Ge C > A at MC2 motif % | SC-C-GS | 0.64 | 0.62 | <0.612951865 | 0.604 |
| cds: A3Bb C > A at MC2 % | T-C-A | 3.11 | 2.72 | <0.609375824 | 2.857 |
| cds: A3Bc G > T at MC1 motif % | T-C-WA | 0.41 | 0.34 | <0.130986459 | 0 |
| cds: A3Bc G > T at MC2 motif % | T-C-WA | 0.27 | 0.21 | <0.073334014 | 0 |
| cds: A3Bd G > A at MC2 motif % | RT-C-A | 0.96 | 0.96 | <0.94942 | 1.227 |
| cds: A3Bd G > A at MC2 cds % | RT-C-A | 0.01 | 0.01 | <0.007215 | 0.009 |
| cds: A3Bf G > T at MC2 % | ST-C-G | 25.93 | 21.80 | <21.06387405 | 37.5 |
| cds: A3Bf G > T at MC2 motif % | ST-C-G | 0.56 | 0.47 | <0.449355721 | 0.674 |
| cds: A3Bh C > A at MC2 % | WT-C-G | 3.18 | 2.63 | <2.838437725 | 9.091 |
| cds: ADAR_Gen1_AAC A > C at MC1 % | -A-AC | 19.05 | 19.02 | <13.97975707 | 16.667 |
| cds: ADAR_Gen1_AAG A > T at MC1 % | -A-AG | 6.37 | 5.16 | <2.739661358 | 11.111 |
| cds: ADAR_Gen1_ACG A > T at MC3 % | -A-CG | 33.18 | 31.39 | <31.49427856 | 40 |
| cds: ADAR_Gen1_AGA T > G at MC2 % | -A-GA | 6.94 | 6.33 | <6.918925 | 7.843 |
| cds: ADAR_Gen1_AGT T > G at MC1 % | -A-GT | 24.82 | 23.02 | <22.86945535 | 26.471 |
| cds: ADAR_Gen1_AGT T > G at MC1 motif % | -A-GT | 1.55 | 1.45 | <1.267918884 | 1.576 |
| cds: ADAR_Gen3_TAA A > C at MC3 % | TA-A- | 3.26 | 2.05 | <1.500788584 | 6.25 |
| cds: ADAR_Gen3_TAA A > T at MC1 % | TA-A- | 27.49 | 25.37 | <27.989285 | 20 |
| cds: ADAR_Gen3_TAA A > C at MC3 motif % | TA-A- | 0.27 | 0.18 | <0.126644354 | 0.524 |
| cds: ADAR_Gen3_TGA A > T at MC3 % | TG-A- | 3.81 | 3.40 | <1.836952575 | 5.882 |
| cds: ADAR_Gen3_TGA A > G at MC3 motif % | TG-A- | 0.51 | 0.50 | <0.039390002 | 0.763 |
| cds: ADAR_Gen3_TGA A > T at MC3 motif % | TG-A- | 0.16 | 0.15 | <0.148483034 | 0.254 |
| cds: ADAR_Gen3_CTA T > G at MC1 % | CT-A- | 1.62 | 0.40 | <1.410163589 | 5.263 |
| cds: ADAR_Gen3_CTA T > G at MC1 motif % | CT-A- | 0.05 | 0.01 | <0.048058096 | 0.208 |
| cds: Gen1_CTA G > C at MC1 % | -C-TA | 33.72 | 32.65 | <19.79331613 | 35.294 |
| cds: Gen1_CAT C > A at MC1 motif % | -C-AT | 1.86 | 1.74 | <1.658936099 | 1.914 |
| cds: Gen3_TAC C > G at MC3 motif % | TA-C- | 0.39 | 0.36 | <0.190921122 | 0.503 |
| cds: Gen3_TAC G > T at MC3 cds % | TA-C- | 0.02 | 0.02 | <0.015858845 | 0.018 |
| cds: Gen3_CGC C > G at MC2 % | CG-C- | 21.76 | 18.25 | <21.62058 | 24 |
| cds: Gen3_CGC C > G at MC2 motif % | CG-C- | 1.27 | 1.07 | <0.85230717 | 1.511 |
| cds: AID MC2 % | WR-C- | 23.06 | 23.14 | >24.01910479 | 23.24 |
| cds: AID G > T at MC1 % | WR-C- | 29.33 | 30.36 | >33.47382304 | 28.235 |
| cds: AID G non-syn % | WR-C- | 58.39 | 58.58 | >60.10701418 | 57.661 |
| cds: Gen2_ACA C > A at MC2 % | A-C-A | 21.66 | 21.87 | >30.6654003 | 20 |
| cds: Gen2_CCA C > A at MC2 % | C-C-A | 17.61 | 17.65 | >23.70730545 | 21.311 |
| cds: ADAR_Gen2_AAA Ti A:T % | A-A-A | 63.53 | 64.27 | >66.66412048 | 63.701 |
| cds: ADAR_Gen2_TAA T > G at MC3 motif % | T-A-A | 4.17 | 4.16 | >5.715045367 | 3.053 |
| cds: ADAR_Gen2_TAT Ti A:T % | T-A-T | 53.12 | 53.30 | >55.89277042 | 53.846 |
| cds: ADAR_Gen2_AAC A > G at MC1 % | A-A-C | 21.17 | 21.73 | >22.78781374 | 21.888 |
| cds: AIDg C > A at MC2 cds % | AG-C-TNT | 0.00 | 0.00 | >0.00024 | 0 |
| cds: A3Ge C > T at MC2 motif % | SC-C-GS | 10.34 | 10.70 | >12.02254336 | 10.574 |
| cds: A3Gi G > C at MC2 % | SG-C-G | 14.08 | 14.88 | >21.26907184 | 16.216 |
| cds: A3Bc C > T at MC2 % | T-C-WA | 22.78 | 23.60 | >30.80608018 | 17.073 |
| cds: A3Bc G > C cds % | T-C-WA | 0.07 | 0.07 | >0.06971 | 0.071 |
| cds: A3Bd Ti C:G % | RT-C-A | 51.52 | 52.69 | >58.22735937 | 45.455 |
| cds: A3Bg G > T at MC3 motif % | T-C-GA | 0.24 | 0.42 | >0.551833367 | 0 |
| cds: A3Bg G > T at MC3 cds % | T-C-GA | 0.00 | 0.00 | >0.004351324 | 0 |
| cds: ADAR_Gen1_AAG Ti A:T % | -A-AG | 52.21 | 52.69 | >53.51901206 | 50.919 |
| cds: ADAR_Gen1_ACG A > T % | -A-CG | 6.13 | 6.62 | >7.301827184 | 3.571 |
| cds: ADAR_Gen1_ACG A > T at MC2 motif % | -A-CG | 1.35 | 1.50 | >2.211404266 | 0.687 |
| cds: ADAR_Gen3_ATA Ti A:T % | AT-A- | 40.15 | 40.54 | >40.4023047 | 40.611 |
| cds: ADAR_Gen3_CAA A > G at MC1 % | CA-A- | 20.29 | 20.76 | >22.63003594 | 20.149 |
| cds: ADAR_Gen3_GTA T > A at MC1 motif % | GT-A- | 1.09 | 1.14 | >1.448900265 | 0.826 |
| cds: Gen1_CAT C > T at MC1 cds % | -C-AT | 0.13 | 0.13 | >0.154483188 | 0.129 |
| cds: Gen1_CGC C > G at MC1 % | -C-GC | 24.49 | 24.54 | >31.79870841 | 30.435 |
| cds: Gen1_CCG G > T at MC1 % | -C-CG | 20.09 | 21.41 | >31.12662974 | 21.739 |
| cds: Gen1_CCG G > T at MC1 motif % | -C-CG | 1.76 | 1.91 | >2.875888918 | 1.792 |
| cds: Gen3_TCC C > G % | TC-C- | 15.16 | 15.79 | >17.24642699 | 13.986 |
| cds: Gen3_CGC G > C % | CG-C- | 16.77 | 16.99 | >20.10457193 | 14.286 |
| cds: Gen3_CGC C > A at MC2 % | CG-C- | 24.48 | 24.96 | >30.13387147 | 26.087 |
| cds: Gen3_CGC C > G at MC1 % | CG-C- | 24.97 | 25.43 | >36.83480846 | 20 |
| cds: Gen3_CGC C > A at MC2 motif % | CG-C- | 1.58 | 1.60 | >2.534077779 | 1.511 |
| cds: Gen3_CGC C > G at MC1 motif % | CG-C- | 1.44 | 1.46 | >2.182833279 | 1.259 |
| cds: Gen3_CGC C > A at MC2 cds % | CG-C- | 0.03 | 0.03 | >0.045609449 | 0.027 |
| cds: Gen3_CGC C > G at MC1 cds % | CG-C- | 0.03 | 0.03 | >0.039173192 | 0.022 |
| variants in VCF | NA | 3442333 | 3445358 | <3382123.992 | 3408356 |
| cds: CDS Variants | NA | 22634 | 22652 | <22146.55666 | 22522 |
| cds: ADAR_Gen1_AAG A > C at MC1 cds % | -A-AG | 0.10 | 0.10 | <0.072485463 | 0.102 |
| cds: ADAR_Gen1_ATC A > G at MC1 cds % | -A-TC | 0.53 | 0.53 | <0.470053735 | 0.511 |
| cds: ADAR_Gen1_ATG A > T at MC1 cds % | -A-TG | 0.08 | 0.09 | <0.05765421 | 0.084 |
| cds: Gen1_CAG C > T at MC1 cds % | -C-AG | 0.09 | 0.09 | <0.059069508 | 0.08 |
| cds: Gen1_CCC C > T at MC1 cds % | -C-CC | 0.29 | 0.29 | <0.238077873 | 0.302 |
| cds: Gen1_CGC C > A at MC1 cds % | -C-GC | 0.05 | 0.05 | <0.028673508 | 0.058 |
| cds: Gen1_CGC C > T at MC1 cds % | -C-GC | 0.43 | 0.43 | <0.364148268 | 0.404 |
| cds: Gen1_CGC C > G at MC1 cds % | -C-GC | 0.06 | 0.06 | <0.036900549 | 0.062 |
| cds: Gen1_CGG C > T at MC1 cds % | -C-GG | 0.52 | 0.52 | <0.451486114 | 0.595 |
| cds: Gen1_CTC C > G at MC1 cds % | -C-TC | 0.11 | 0.11 | <0.077924319 | 0.084 |
| cds: Gen1_CTT C > T at MC1 cds % | -C-TT | 0.11 | 0.11 | <0.076607155 | 0.124 |
| cds: Gen3_GTC G > A at MC1 cds % | GT-C- | 0.27 | 0.26 | <0.216293012 | 0.306 |
| cds: Gen3_CTC G > A at MC1 cds % | CT-C- | 0.38 | 0.39 | <0.32217631 | 0.4 |
| cds: Gen3_ATC G > A at MC1 cds % | AT-C- | 0.24 | 0.24 | <0.192871809 | 0.258 |
| cds: Gen3_CCC G > C at MC1 cds % | CC-C- | 0.11 | 0.11 | <0.080011213 | 0.098 |
| cds: Gen3_CCC G > A at MC1 cds % | CC-C- | 0.30 | 0.30 | <0.250428325 | 0.258 |
| cds: Gen3_GAC G > T at MC1 cds % | GA-C- | 0.04 | 0.04 | <0.016028166 | 0.027 |
| cds: Gen3_CAC G > T at MC1 cds % | CA-C- | 0.10 | 0.11 | <0.075177963 | 0.102 |
| cds: Gen3_CAC G > A at MC1 cds % | CA-C- | 0.74 | 0.73 | <0.666327506 | 0.737 |
| cds: Gen3_AAC G > A at MC1 cds % | AA-C- | 0.39 | 0.39 | <0.335821091 | 0.351 |
| cds: ADAR_Gen3_GCA T > C at MC1 cds % | GC-A- | 0.28 | 0.28 | <0.247743322 | 0.289 |
| cds: ADAR_Gen3_AAA T > A at MC1 cds % | AA-A- | 0.04 | 0.04 | <0.025602893 | 0.049 |
| cds: ADAR_Gen2_AAA A > T at MC2 cds % | A-A-A | 0.02 | 0.02 | <0.007675401 | 0.013 |
| cds: ADAR_Gen2_AAC A > T at MC2 cds % | A-A-C | 0.03 | 0.03 | <0.019257836 | 0.022 |
| cds: Gen2_ACA C > T at MC2 cds % | A-C-A | 0.23 | 0.22 | <0.185713373 | 0.195 |
| cds: Gen2_ACG C > G at MC2 cds % | A-C-G | 0.05 | 0.05 | <0.031246337 | 0.044 |
| cds: Gen2_TCT G > C at MC2 cds % | T-C-T | 0.06 | 0.06 | <0.042219322 | 0.071 |
| cds: Gen2_TCT G > T at MC2 cds % | T-C-T | 0.02 | 0.02 | <0.006970146 | 0.018 |
| cds: Gen2_ACT G > A at MC2 cds % | A-C-T | 0.33 | 0.33 | <0.290455886 | 0.355 |
| cds: ADAR_Gen2_CAT A > G at MC2 cds % | C-A-T | 0.38 | 0.37 | <0.333345954 | 0.36 |
| cds: Gen2_TCG G > A at MC2 cds % | T-C-G | 0.40 | 0.40 | <0.343487378 | 0.444 |
| cds: Gen2_GCG G > T at MC2 cds % | G-C-G | 0.06 | 0.06 | <0.03884469 | 0.08 |
| cds: Gen2_CCG G > A at MC2 cds % | C-C-G | 0.81 | 0.81 | <0.723556705 | 0.795 |
| cds: Gen2_ACG G > C at MC2 cds % | A-C-G | 0.05 | 0.05 | <0.035765884 | 0.049 |
| cds: ADAR_Gen2_CAG T > C at MC2 cds % | C-A-G | 0.48 | 0.49 | <0.435830748 | 0.453 |
| cds: ADAR_Gen2_AAG T > C at MC2 cds % | A-A-G | 0.15 | 0.15 | <0.126168085 | 0.169 |
| cds: ADAR_Gen2_GAC A > C at MC2 cds % | G-A-C | 0.07 | 0.07 | <0.048150124 | 0.067 |
| cds: ADAR_Gen2_GAC A > T at MC2 cds % | G-A-C | 0.03 | 0.03 | <0.010939551 | 0.018 |
| cds: ADAR_Gen2_GAC A > G at MC2 cds % | G-A-C | 0.18 | 0.18 | <0.152006077 | 0.204 |
| cds: ADAR_Gen2_GAG A > C at MC2 cds % | G-A-G | 0.07 | 0.08 | <0.050409603 | 0.084 |
| cds: Gen2_GCA C > A at MC2 cds % | G-C-A | 0.09 | 0.09 | <0.068042379 | 0.107 |
| cds: Gen2_GCC C > A at MC2 cds % | G-C-C | 0.08 | 0.08 | <0.054211463 | 0.089 |
| cds: Gen2_GCG C > A at MC2 cds % | G-C-G | 0.06 | 0.06 | <0.038015023 | 0.053 |
| cds: Gen2_GCT C > T at MC2 cds % | G-C-T | 0.15 | 0.15 | <0.119253343 | 0.173 |
| cds: Gen2_GCC G > T at MC2 cds % | G-C-C | 0.07 | 0.07 | <0.046700735 | 0.071 |
| cds: Gen2_CCC G > A at MC2 cds % | C-C-C | 0.21 | 0.21 | <0.167018169 | 0.2 |
| cds: ADAR_Gen2_CAC T > A at MC2 cds % | C-A-C | 0.04 | 0.04 | <0.023798003 | 0.04 |
| cds: ADAR_Gen2_CAC T > C at MC2 cds % | C-A-C | 0.51 | 0.52 | <0.461907775 | 0.511 |
| cds: ADAR_Gen2_TAT A > G at MC2 cds % | T-A-T | 0.17 | 0.18 | <0.133539846 | 0.195 |
| cds: Gen2_TCT C > T at MC2 cds % | T-C-T | 0.08 | 0.08 | <0.056814621 | 0.062 |
| cds: Gen2_CCA G > A at MC2 cds % | C-C-A | 0.05 | 0.05 | <0.027005578 | 0.044 |
| cds: ADAR_Gen2_GAA T > A at MC2 cds % | G-A-A | 0.05 | 0.05 | <0.026891486 | 0.062 |
| cds: ADAR_Gen3_AAA A > T at MC3 cds % | AA-A- | 0.05 | 0.05 | <0.031805586 | 0.049 |
| cds: Gen3_ATC C > G at MC3 cds % | AT-C- | 0.08 | 0.08 | <0.059003633 | 0.075 |
| cds: Gen1_CAT G > A at MC3 cds % | -C-AT | 0.20 | 0.20 | <0.161563009 | 0.191 |
| cds: Gen3_CAC C > A at MC3 cds % | CA-C- | 0.06 | 0.06 | <0.043459322 | 0.075 |
| cds: Gen1_CTG G > C at MC3 cds % | -C-TG | 0.14 | 0.14 | <0.107493229 | 0.133 |
| cds: ADAR_Gen1_ATG T > G at MC3 cds % | -A-TG | 0.07 | 0.08 | <0.0556974 | 0.058 |
| cds: Gen3_GAC C > G at MC3 cds % | GA-C- | 0.15 | 0.15 | <0.118560855 | 0.147 |
| cds: Gen1_CTG G > C at MC3 cds % | -C-TG | 0.14 | 0.14 | <0.107493229 | 0.133 |
| cds: ADAR_Gen1_ATA T > G at MC3 cds % | -A-TA | 0.02 | 0.02 | <0.010498433 | 0.022 |
| cds: Gen3_TTC C > A at MC3 cds % | TT-C- | 0.04 | 0.04 | <0.022749906 | 0.049 |
| variants in VCF | NA | 3442333 | 3445358 | >3502542 | 3408356 |
| cds: CDS Variants | NA | 22634 | 22652 | >23121 | 22522 |
| cds: ADAR_Gen1_AAG A > C at MC1 cds % | -A-AG | 0.10 | 0.10 | >0.125560691 | 0.102 |
| cds: ADAR_Gen1_ATC A > G at MC1 cds % | -A-TC | 0.53 | 0.53 | >0.58083088 | 0.511 |
| cds: ADAR_Gen1_ATG A > T at MC1 cds % | -A-TG | 0.08 | 0.09 | >0.108184252 | 0.084 |
| cds: Gen1_CAG C > T at MC1 cds % | -C-AG | 0.09 | 0.09 | >0.112268953 | 0.08 |
| cds: Gen1_CCC C > T at MC1 cds % | -C-CC | 0.29 | 0.29 | >0.345368281 | 0.302 |
| cds: Gen1_CGC C > A at MC1 cds % | -C-GC | 0.05 | 0.05 | >0.068226492 | 0.058 |
| cds: Gen1_CGC C > T at MC1 cds % | -C-GC | 0.43 | 0.43 | >0.489328655 | 0.404 |
| cds: Gen1_CGC C > G at MC1 cds % | -C-GC | 0.06 | 0.06 | >0.074899451 | 0.062 |
| cds: Gen1_CGG C > T at MC1 cds % | -C-GG | 0.52 | 0.52 | >0.590152348 | 0.595 |
| cds: Gen1_CTC C > G at MC1 cds % | -C-TC | 0.11 | 0.11 | >0.134698758 | 0.084 |
| cds: Gen1_CTT C > T at MC1 cds % | -C-TT | 0.11 | 0.11 | >0.140292845 | 0.124 |
| cds: Gen3_GTC G > A at MC1 cds % | GT-C- | 0.27 | 0.26 | >0.321537757 | 0.306 |
| cds: Gen3_CTC G > A at MC1 cds % | CT-C- | 0.38 | 0.39 | >0.441585228 | 0.4 |
| cds: Gen3_ATC G > A at MC1 cds % | AT-C- | 0.24 | 0.24 | >0.282735883 | 0.258 |
| cds: Gen3_CCC G > C at MC1 cds % | CC-C- | 0.11 | 0.11 | >0.13399648 | 0.098 |
| cds: Gen3_CCC G > A at MC1 cds % | CC-C- | 0.30 | 0.30 | >0.347540906 | 0.258 |
| cds: Gen3_GAC G > T at MC1 cds % | GA-C- | 0.04 | 0.04 | >0.054287218 | 0.027 |
| cds: Gen3_CAC G > T at MC1 cds % | CA-C- | 0.10 | 0.11 | >0.13269896 | 0.102 |
| cds: Gen3_CAC G > A at MC1 cds % | CA-C- | 0.74 | 0.73 | >0.820380186 | 0.737 |
| cds: Gen3_AAC G > A at MC1 cds % | AA-C- | 0.39 | 0.39 | >0.436755832 | 0.351 |
| cds: ADAR_Gen3_GCA T > C at MC1 cds % | GC-A- | 0.28 | 0.28 | >0.314825909 | 0.289 |
| cds: ADAR_Gen3_AAA T > A at MC1 cds % | AA-A- | 0.04 | 0.04 | >0.056458645 | 0.049 |
| cds: ADAR_Gen2_AAA A > T at MC2 cds % | A-A-A | 0.02 | 0.02 | >0.036455369 | 0.013 |
| cds: ADAR_Gen2_AAC A > T at MC2 cds % | A-A-C | 0.03 | 0.03 | >0.047272933 | 0.022 |
| cds: Gen2_ACA C > T at MC2 cds % | A-C-A | 0.23 | 0.22 | >0.264655858 | 0.195 |
| cds: Gen2_ACG C > G at MC2 cds % | A-C-G | 0.05 | 0.05 | >0.062422894 | 0.044 |
| cds: Gen2_TCT G > C at MC2 cds % | T-C-T | 0.06 | 0.06 | >0.082396063 | 0.071 |
| cds: Gen2_TCT G > T at MC2 cds % | T-C-T | 0.02 | 0.02 | >0.037791393 | 0.018 |
| cds: Gen2_ACT G > A at MC2 cds % | A-C-T | 0.33 | 0.33 | >0.366774883 | 0.355 |
| cds: ADAR_Gen2_CAT A > G at MC2 cds % | C-A-T | 0.38 | 0.37 | >0.41946943 | 0.36 |
| cds: Gen2_TCG G > A at MC2 cds % | T-C-G | 0.40 | 0.40 | >0.455028007 | 0.444 |
| cds: Gen2_GCG G > T at MC2 cds % | G-C-G | 0.06 | 0.06 | >0.088324541 | 0.08 |
| cds: Gen2_CCG G > A at MC2 cds % | C-C-G | 0.81 | 0.81 | >0.898274064 | 0.795 |
| cds: Gen2_ACG G > C at MC2 cds % | A-C-G | 0.05 | 0.05 | >0.068018731 | 0.049 |
| cds: ADAR_Gen2_CAG T > C at MC2 cds % | C-A-G | 0.48 | 0.49 | >0.521776945 | 0.453 |
| cds: ADAR_Gen2_AAG T > C at MC2 cds % | A-A-G | 0.15 | 0.15 | >0.176270377 | 0.169 |
| cds: ADAR_Gen2_GAC A > C at MC2 cds % | G-A-C | 0.07 | 0.07 | >0.083972953 | 0.067 |
| cds: ADAR_Gen2_GAC A > T at MC2 cds % | G-A-C | 0.03 | 0.03 | >0.04089891 | 0.018 |
| cds: ADAR_Gen2_GAC A > G at MC2 cds % | G-A-C | 0.18 | 0.18 | >0.209917 | 0.204 |
| cds: ADAR_Gen2_GAG A > C at MC2 cds % | G-A-G | 0.07 | 0.08 | >0.099151935 | 0.084 |
| cds: Gen2_GCA C > A at MC2 cds % | G-C-A | 0.09 | 0.09 | >0.112634544 | 0.107 |
| cds: Gen2_GCC C > A at MC2 cds % | G-C-C | 0.08 | 0.08 | >0.103388537 | 0.089 |
| cds: Gen2_GCG C > A at MC2 cds % | G-C-G | 0.06 | 0.06 | >0.087377285 | 0.053 |
| cds: Gen2_GCT C > T at MC2 cds % | G-C-T | 0.15 | 0.15 | >0.182815887 | 0.173 |
| cds: Gen2_GCC G > T at MC2 cds % | G-C-C | 0.07 | 0.07 | >0.09411465 | 0.071 |
| cds: Gen2_CCC G > A at MC2 cds % | C-C-C | 0.21 | 0.21 | >0.243720292 | 0.2 |
| cds: ADAR_Gen2_CAC T > A at MC2 cds % | C-A-C | 0.04 | 0.04 | >0.05190969 | 0.04 |
| cds: ADAR_Gen2_CAC T > C at MC2 cds % | C-A-C | 0.51 | 0.52 | >0.561999917 | 0.511 |
| cds: ADAR_Gen2_TAT A > G at MC2 cds % | T-A-T | 0.17 | 0.18 | >0.210060154 | 0.195 |
| cds: Gen2_TCT C > T at MC2 cds % | T-C-T | 0.08 | 0.08 | >0.108393071 | 0.062 |
| cds: Gen2_CCA G > A at MC2 cds % | C-C-A | 0.05 | 0.05 | >0.062994422 | 0.044 |
| cds: ADAR_Gen2_GAA T > A at MC2 cds % | G-A-A | 0.05 | 0.05 | >0.065800822 | 0.062 |
| cds: ADAR_Gen3_AAA A > T at MC3 cds % | AA-A- | 0.05 | 0.05 | >0.063609799 | 0.049 |
| cds: Gen3_ATC C > G at MC3 cds % | AT-C- | 0.08 | 0.08 | >0.101357906 | 0.075 |
| cds: Gen1_CAT G > A at MC3 cds % | -C-AT | 0.20 | 0.20 | >0.241006222 | 0.191 |
| cds: Gen3_CAC C > A at MC3 cds % | CA-C- | 0.06 | 0.06 | >0.085694524 | 0.075 |
| cds: Gen1_CTG G > C at MC3 cds % | -C-TG | 0.14 | 0.14 | >0.16716831 | 0.133 |
| cds: ADAR_Gen1_ATG T > G at MC3 cds % | -A-TG | 0.07 | 0.08 | >0.088571831 | 0.058 |
| cds: Gen3_GAC C > G at MC3 cds % | GA-C- | 0.15 | 0.15 | >0.180839145 | 0.147 |
| cds: Gen1_CTG G > C at MC3 cds % | -C-TG | 0.14 | 0.14 | >0.16716831 | 0.133 |
| cds: ADAR_Gen1_ATA T > G at MC3 cds % | -A-TA | 0.02 | 0.02 | >0.029170798 | 0.022 |
| cds: Gen3_TTC C > A at MC3 cds % | TT-C- | 0.04 | 0.04 | >0.054388555 | 0.049 |
| Total Scores: | | | | | |

| Metric | S* | 4612_CN | S* | 2403_EMCI | S* | 2263_EMCI | S* |
|---|---|---|---|---|---|---|---|
| cds: AID Hits | 1 | 3170 | 0 | 3057 | 1 | 3042 | 1 |
| cds: Gen2_TCA C > A at MC2 % | 0 | 7.317 | 0 | 2.381 | 0 | 2.857 | 0 |
| cds: Gen2_TCT G > T at MC1 % | 1 | 18.519 | 1 | 20 | 1 | 29.412 | 0 |
| cds: Gen2_TCT G > T at MC1 motif % | 1 | 0.943 | 1 | 1.176 | 0 | 1.022 | 1 |
| cds: Gen2_TCC G > T at MC2 % | 0 | 25 | 0 | 15.789 | 1 | 21.875 | 1 |
| cds: Gen2_TCG G > T at MC2 % | 0 | 13.043 | 1 | 10 | 1 | 11.111 | 1 |
| cds: ADAR_Gen2_TAA T > G at MC1 % | 0 | 28.571 | 0 | 25 | 1 | 21.053 | 1 |
| cds: AIDe G > T at MC2 motif % | 0 | 0.482 | 0 | 0.175 | 0 | 0 | 1 |
| cds: ADARe A > C at MC1 % | 0 | 20 | 0 | 20 | 0 | 9.091 | 1 |
| cds: ADARj T > G at MC2 % | 0 | 6.977 | 1 | 11.905 | 0 | 6.977 | 1 |
| cds: A3Gd G > C at MC2 motif % | 0 | 0.211 | 1 | 0.455 | 0 | 0.466 | 0 |
| cds: A3Ge C > A at MC2 % | 0 | 17.857 | 0 | 11.765 | 1 | 12.121 | 1 |
| cds: A3Ge C > A at MC2 motif % | 1 | 0.742 | 0 | 0.593 | 1 | 0.613 | 0 |
| cds: A3Bb C > A at MC2 % | 0 | 7.317 | 0 | 2.381 | 0 | 2.857 | 0 |
| cds: A3Bc G > T at MC1 motif % | 1 | 0.752 | 0 | 0 | 1 | 0 | 1 |
| cds: A3Bc G > T at MC2 motif % | 1 | 0 | 1 | 0.781 | 0 | 0 | 1 |
| cds: A3Bd G > A at MC2 motif % | 0 | 1.63 | 0 | 1.754 | 0 | 1.205 | 0 |
| cds: A3Bd G > A at MC2 cds % | 0 | 0.013 | 0 | 0.013 | 0 | 0.009 | 0 |
| cds: A3Bf G > T at MC2 % | 0 | 15.385 | 1 | 16.667 | 1 | 20 | 1 |
| cds: A3Bf G > T at MC2 motif % | 0 | 0.43 | 1 | 0.442 | 1 | 0.48 | 0 |
| cds: A3Bh C > A at MC2 % | 0 | 0 | 1 | 8.333 | 0 | 7.692 | 0 |
| cds: ADAR_Gen1_AAC A > C at MC1 % | 0 | 15.789 | 0 | 17.647 | 0 | 20.69 | 0 |
| cds: ADAR_Gen1_AAG A > T at MC1 % | 0 | 0 | 1 | 0 | 1 | 0 | 1 |
| cds: ADAR_Gen1_ACG A > T at MC3 % | 0 | 28.571 | 1 | 30 | 1 | 30 | 1 |
| cds: ADAR_Gen1_AGA T > G at MC2 % | 0 | 5.556 | 1 | 9.091 | 0 | 4.348 | 1 |
| cds: ADAR_Gen1_AGT T > G at MC1 % | 0 | 26.471 | 0 | 14.706 | 1 | 16.667 | 1 |
| cds: ADAR_Gen1_AGT T > G at MC1 motif % | 0 | 1.471 | 0 | 0.833 | 1 | 1.058 | 1 |
| cds: ADAR_Gen3_TAA A > C at MC3 % | 0 | 6.667 | 0 | 0 | 1 | 0 | 1 |
| cds: ADAR_Gen3_TAA A > T at MC1 % | 1 | 22.222 | 1 | 16.667 | 1 | 22.222 | 1 |
| cds: ADAR_Gen3_TAA A > C at MC3 motif % | 0 | 0.5 | 0 | 0 | 1 | 0 | 1 |
| cds: ADAR_Gen3_TGA A > T at MC3 % | 0 | 11.765 | 0 | 0 | 1 | 0 | 1 |
| cds: ADAR_Gen3_TGA A > G at MC3 motif % | 0 | 0.512 | 0 | 0.262 | 0 | 0.506 | 0 |
| cds: ADAR_Gen3_TGA A > T at MC3 motif % | 0 | 0.512 | 0 | 0 | 1 | 0 | 1 |
| cds: ADAR_Gen3_CTA T > G at MC1 % | 0 | 6.25 | 0 | 0 | 1 | 0 | 1 |
| cds: ADAR_Gen3_CTA T > G at MC1 motif % | 0 | 0.214 | 0 | 0 | 1 | 0 | 1 |
| cds: Gen1_CTA G > C at MC1 % | 0 | 42.105 | 0 | 38.095 | 0 | 33.333 | 0 |
| cds: Gen1_CAT C > A at MC1 motif % | 0 | 2.387 | 0 | 1.474 | 1 | 1.511 | 1 |
| cds: Gen3_TAC C > G at MC3 motif % | 0 | 0.502 | 0 | 0.18 | 1 | 0.525 | 0 |
| cds: Gen3_TAC G > T at MC3 cds % | 0 | 0.035 | 0 | 0.018 | 0 | 0.018 | 0 |
| cds: Gen3_CGC C > G at MC2 % | 0 | 16 | 1 | 23.077 | 0 | 8 | 1 |
| cds: Gen3_CGC C > G at MC2 motif % | 0 | 0.98 | 0 | 1.446 | 0 | 0.474 | 1 |
| cds: AID MC2 % | 0 | 23.85 | 0 | 23.36 | 0 | 23.27 | 0 |
| cds: AID G > T at MC1 % | 0 | 27.225 | 0 | 28.736 | 0 | 33.514 | 1 |
| cds: AID G non-syn % | 0 | 59.466 | 0 | 58.11 | 0 | 58.887 | 0 |
| cds: Gen2_ACA C > A at MC2 % | 0 | 17.5 | 0 | 26.087 | 0 | 31.034 | 1 |
| cds: Gen2_CCA C > A at MC2 % | 0 | 20 | 0 | 18.462 | 0 | 16.129 | 0 |
| cds: ADAR_Gen2_AAA Ti A:T % | 0 | 64.262 | 0 | 61.074 | 0 | 61.433 | 0 |
| cds: ADAR_Gen2_TAA T > G at MC3 motif % | 0 | 3.383 | 0 | 4.059 | 0 | 4.965 | 0 |
| cds: ADAR_Gen2_TAT Ti A:T % | 0 | 52.618 | 0 | 52.956 | 0 | 54.937 | 0 |
| cds: ADAR_Gen2_AAC A > G at MC1 % | 0 | 22.433 | 0 | 22.273 | 0 | 23.265 | 1 |
| cds: AIDg C > A at MC2 cds % | 0 | 0 | 0 | 0 | 0 | 0 | 0 |
| cds: A3Ge C > T at MC2 motif % | 0 | 10.386 | 0 | 10.682 | 0 | 9.969 | 0 |
| cds: A3Gi G > C at MC2 % | 0 | 8.824 | 0 | 14.706 | 0 | 13.793 | 0 |
| cds: A3Bc C > T at MC2 % | 0 | 22.727 | 0 | 29.412 | 0 | 22.222 | 0 |
| cds: A3Bc G > C cds % | 1 | 0.061 | 0 | 0.075 | 1 | 0.062 | 0 |
| cds: A3Bd Ti C:G % | 0 | 54.206 | 0 | 52.083 | 0 | 53.846 | 0 |
| cds: A3Bg G > T at MC3 motif % | 0 | 0.521 | 0 | 0.515 | 0 | 1.031 | 1 |
| cds: A3Bg G > T at MC3 cds % | 0 | 0.004 | 0 | 0.004 | 0 | 0.009 | 1 |
| cds: ADAR_Gen1_AAG Ti A:T % | 0 | 53.253 | 0 | 51.378 | 0 | 49.749 | 0 |
| cds: ADAR_Gen1_ACG A > T % | 0 | 4.459 | 0 | 7.194 | 0 | 7.042 | 0 |
| cds: ADAR_Gen1_ACG A > T at MC2 motif % | 0 | 1.316 | 0 | 1.678 | 0 | 1.375 | 0 |
| cds: ADAR_Gen3_ATA Ti A:T % | 1 | 40.435 | 1 | 40.773 | 1 | 38.938 | 0 |
| cds: ADAR_Gen3_CAA A > G at MC1 % | 0 | 20.244 | 0 | 19.588 | 0 | 20.11 | 0 |
| cds: ADAR_Gen3_GTA T > A at MC1 motif % | 0 | 0.75 | 0 | 0.787 | 0 | 1.323 | 0 |
| cds: Gen1_CAT C > T at MC1 cds % | 0 | 0.131 | 0 | 0.155 | 1 | 0.137 | 0 |
| cds: Gen1_CGC C > G at MC1 % | 0 | 29.167 | 0 | 22.642 | 0 | 19.231 | 0 |
| cds: Gen1_CCG G > T at MC1 % | 0 | 30.769 | 0 | 28.571 | 0 | 17.391 | 0 |
| cds: Gen1_CCG G > T at MC1 motif % | 0 | 2.827 | 0 | 2.062 | 0 | 1.404 | 0 |
| cds: Gen3_TCC C > G % | 0 | 17.266 | 1 | 15.686 | 0 | 15 | 0 |
| cds: Gen3_CGC G > C % | 0 | 19.807 | 0 | 14.925 | 0 | 16.74 | 0 |
| cds: Gen3_CGC C > A at MC2 % | 0 | 16 | 0 | 24.138 | 0 | 40.741 | 1 |
| cds: Gen3_CGC C > G at MC1 % | 0 | 24 | 0 | 26.923 | 0 | 32 | 0 |
| cds: Gen3_CGC C > A at MC2 motif % | 0 | 0.98 | 0 | 1.687 | 0 | 2.607 | 1 |
| cds: Gen3_CGC C > G at MC1 motif % | 0 | 1.471 | 0 | 1.687 | 0 | 1.896 | 0 |
| cds: Gen3_CGC C > A at MC2 cds % | 0 | 0.017 | 0 | 0.031 | 0 | 0.049 | 1 |
| cds: Gen3_CGC C > G at MC1 cds % | 0 | 0.026 | 0 | 0.031 | 0 | 0.035 | 0 |
| variants in VCF | 0 | 3455139 | 0 | 3451913 | 0 | 3421362 | 0 |
| cds: CDS Variants | 0 | 22969 | 0 | 22620 | 0 | 22614 | 0 |
| cds: ADAR_Gen1_AAG A > C at MC1 cds % | 0 | 0.074 | 0 | 0.071 | 1 | 0.071 | 1 |
| cds: ADAR_Gen1_ATC A > G at MC1 cds % | 0 | 0.514 | 0 | 0.535 | 0 | 0.522 | 0 |
| cds: ADAR_Gen1_ATG A > T at MC1 cds % | 0 | 0.091 | 0 | 0.084 | 0 | 0.097 | 0 |
| cds: Gen1_CAG C > T at MC1 cds % | 0 | 0.096 | 0 | 0.106 | 0 | 0.084 | 0 |
| cds: Gen1_CCC C > T at MC1 cds % | 0 | 0.292 | 0 | 0.336 | 0 | 0.314 | 0 |
| cds: Gen1_CGC C > A at MC1 cds % | 0 | 0.044 | 0 | 0.049 | 0 | 0.049 | 0 |
| cds: Gen1_CGC C > T at MC1 cds % | 0 | 0.444 | 0 | 0.455 | 0 | 0.38 | 0 |
| cds: Gen1_CGC C > G at MC1 cds % | 0 | 0.061 | 0 | 0.053 | 0 | 0.044 | 0 |
| cds: Gen1_CGG C > T at MC1 cds % | 0 | 0.479 | 0 | 0.469 | 0 | 0.531 | 0 |
| cds: Gen1_CTC C > G at MC1 cds % | 0 | 0.104 | 0 | 0.111 | 0 | 0.124 | 0 |
| cds: Gen1_CTT C > T at MC1 cds % | 0 | 0.126 | 0 | 0.115 | 0 | 0.106 | 0 |
| cds: Gen3_GTC G > A at MC1 cds % | 0 | 0.261 | 0 | 0.296 | 0 | 0.256 | 0 |
| cds: Gen3_CTC G > A at MC1 cds % | 0 | 0.405 | 0 | 0.34 | 0 | 0.398 | 0 |
| cds: Gen3_ATC G > A at MC1 cds % | 0 | 0.222 | 0 | 0.248 | 0 | 0.186 | 1 |
| cds: Gen3_CCC G > C at MC1 cds % | 0 | 0.104 | 0 | 0.093 | 0 | 0.093 | 0 |
| cds: Gen3_CCC G > A at MC1 cds % | 0 | 0.283 | 0 | 0.265 | 0 | 0.323 | 0 |
| cds: Gen3_GAC G > T at MC1 cds % | 0 | 0.044 | 0 | 0.035 | 0 | 0.027 | 0 |
| cds: Gen3_CAC G > T at MC1 cds % | 0 | 0.104 | 0 | 0.128 | 0 | 0.093 | 0 |
| cds: Gen3_CAC G > A at MC1 cds % | 0 | 0.749 | 0 | 0.698 | 0 | 0.725 | 0 |
| cds: Gen3_AAC G > A at MC1 cds % | 0 | 0.431 | 0 | 0.389 | 0 | 0.354 | 0 |
| cds: ADAR_Gen3_GCA T > C at MC1 cds % | 0 | 0.27 | 0 | 0.296 | 0 | 0.301 | 0 |
| cds: ADAR_Gen3_AAA T > A at MC1 cds % | 0 | 0.048 | 0 | 0.044 | 0 | 0.044 | 0 |
| cds: ADAR_Gen2_AAA A > T at MC2 cds % | 0 | 0.03 | 0 | 0.031 | 0 | 0.013 | 0 |
| cds: ADAR_Gen2_AAC A > T at MC2 cds % | 0 | 0.039 | 0 | 0.031 | 0 | 0.031 | 0 |
| cds: Gen2_ACA C > T at MC2 cds % | 0 | 0.235 | 0 | 0.212 | 0 | 0.265 | 0 |
| cds: Gen2_ACG C > G at MC2 cds % | 0 | 0.039 | 0 | 0.053 | 0 | 0.04 | 0 |
| cds: Gen2_TCT G > C at MC2 cds % | 0 | 0.07 | 0 | 0.049 | 0 | 0.053 | 0 |
| cds: Gen2_TCT G > T at MC2 cds % | 0 | 0.026 | 0 | 0.035 | 0 | 0.018 | 0 |
| cds: Gen2_ACT G > A at MC2 cds % | 0 | 0.309 | 0 | 0.323 | 0 | 0.327 | 0 |
| cds: ADAR_Gen2_CAT A > G at MC2 cds % | 0 | 0.37 | 0 | 0.332 | 1 | 0.332 | 1 |
| cds: Gen2_TCG G > A at MC2 cds % | 0 | 0.414 | 0 | 0.358 | 0 | 0.389 | 0 |
| cds: Gen2_GCG G > T at MC2 cds % | 0 | 0.078 | 0 | 0.066 | 0 | 0.044 | 0 |
| cds: Gen2_CCG G > A at MC2 cds % | 0 | 0.771 | 0 | 0.765 | 0 | 0.8 | 0 |
| cds: Gen2_ACG G > C at MC2 cds % | 0 | 0.052 | 0 | 0.049 | 0 | 0.053 | 0 |
| cds: ADAR_Gen2_CAG T > C at MC2 cds % | 0 | 0.466 | 0 | 0.522 | 0 | 0.469 | 0 |
| cds: ADAR_Gen2_AAG T > C at MC2 cds % | 0 | 0.144 | 0 | 0.172 | 0 | 0.133 | 0 |
| cds: ADAR_Gen2_GAC A > C at MC2 cds % | 0 | 0.061 | 0 | 0.075 | 0 | 0.071 | 0 |
| cds: ADAR_Gen2_GAC A > T at MC2 cds % | 0 | 0.039 | 0 | 0.027 | 0 | 0.027 | 0 |
| cds: ADAR_Gen2_GAC A > G at MC2 cds % | 0 | 0.192 | 0 | 0.159 | 0 | 0.181 | 0 |
| cds: ADAR_Gen2_GAG A > C at MC2 cds % | 0 | 0.074 | 0 | 0.066 | 0 | 0.066 | 0 |
| cds: Gen2_GCA C > A at MC2 cds % | 0 | 0.087 | 0 | 0.115 | 0 | 0.071 | 0 |
| cds: Gen2_GCC C > A at MC2 cds % | 0 | 0.1 | 0 | 0.088 | 0 | 0.097 | 0 |
| cds: Gen2_GCG C > A at MC2 cds % | 0 | 0.039 | 0 | 0.057 | 0 | 0.071 | 0 |
| cds: Gen2_GCT C > T at MC2 cds % | 0 | 0.148 | 0 | 0.15 | 0 | 0.172 | 0 |
| cds: Gen2_GCC G > T at MC2 cds % | 0 | 0.07 | 0 | 0.057 | 0 | 0.075 | 0 |
| cds: Gen2_CCC G > A at MC2 cds % | 0 | 0.205 | 0 | 0.234 | 0 | 0.195 | 0 |
| cds: ADAR_Gen2_CAC T > A at MC2 cds % | 0 | 0.039 | 0 | 0.044 | 0 | 0.031 | 0 |
| cds: ADAR_Gen2_CAC T > C at MC2 cds % | 0 | 0.466 | 0 | 0.491 | 0 | 0.522 | 0 |
| cds: ADAR_Gen2_TAT A > G at MC2 cds % | 0 | 0.165 | 0 | 0.186 | 0 | 0.195 | 0 |
| cds: Gen2_TCT C > T at MC2 cds % | 0 | 0.091 | 0 | 0.071 | 0 | 0.053 | 1 |
| cds: Gen2_CCA G > A at MC2 cds % | 0 | 0.057 | 0 | 0.049 | 0 | 0.049 | 0 |
| cds: ADAR_Gen2_GAA T > A at MC2 cds % | 0 | 0.039 | 0 | 0.035 | 0 | 0.066 | 0 |
| cds: ADAR_Gen3_AAA A > T at MC3 cds % | 0 | 0.035 | 0 | 0.044 | 0 | 0.04 | 0 |
| cds: Gen3_ATC C > G at MC3 cds % | 0 | 0.074 | 0 | 0.084 | 0 | 0.075 | 0 |
| cds: Gen1_CAT G > A at MC3 cds % | 0 | 0.239 | 0 | 0.195 | 0 | 0.181 | 0 |
| cds: Gen3_CAC C > A at MC3 cds % | 0 | 0.03 | 1 | 0.062 | 0 | 0.075 | 0 |
| cds: Gen1_CTG G > C at MC3 cds % | 0 | 0.118 | 0 | 0.097 | 1 | 0.15 | 0 |
| cds: ADAR_Gen1_ATG T > G at MC3 cds % | 0 | 0.083 | 0 | 0.093 | 0 | 0.084 | 0 |
| cds: Gen3_GAC C > G at MC3 cds % | 0 | 0.135 | 0 | 0.133 | 0 | 0.137 | 0 |
| cds: Gen1_CTG G > C at MC3 cds % | 0 | 0.118 | 0 | 0.097 | 1 | 0.15 | 0 |
| cds: ADAR_Gen1_ATA T > G at MC3 cds % | 0 | 0.022 | 0 | 0.009 | 1 | 0.022 | 0 |
| cds: Gen3_TTC C > A at MC3 cds % | 0 | 0.039 | 0 | 0.04 | 0 | 0.035 | 0 |
| variants in VCF | 0 | 3455139 | 0 | 3451913 | 0 | 3421362 | 0 |
| cds: CDS Variants | 0 | 22969 | 0 | 22620 | 0 | 22614 | 0 |
| cds: ADAR_Gen1_AAG A > C at MC1 cds % | 0 | 0.074 | 0 | 0.071 | 0 | 0.071 | 0 |
| cds: ADAR_Gen1_ATC A > G at MC1 cds % | 0 | 0.514 | 0 | 0.535 | 0 | 0.522 | 0 |
| cds: ADAR_Gen1_ATG A > T at MC1 cds % | 0 | 0.091 | 0 | 0.084 | 0 | 0.097 | 0 |
| cds: Gen1_CAG C > T at MC1 cds % | 0 | 0.096 | 0 | 0.106 | 0 | 0.084 | 0 |
| cds: Gen1_CCC C > T at MC1 cds % | 0 | 0.292 | 0 | 0.336 | 0 | 0.314 | 0 |
| cds: Gen1_CGC C > A at MC1 cds % | 0 | 0.044 | 0 | 0.049 | 0 | 0.049 | 0 |
| cds: Gen1_CGC C > T at MC1 cds % | 0 | 0.444 | 0 | 0.455 | 0 | 0.38 | 0 |
| cds: Gen1_CGC C > G at MC1 cds % | 0 | 0.061 | 0 | 0.053 | 0 | 0.044 | 0 |
| cds: Gen1_CGG C > T at MC1 cds % | 1 | 0.479 | 0 | 0.469 | 0 | 0.531 | 0 |
| cds: Gen1_CTC C > G at MC1 cds % | 0 | 0.104 | 0 | 0.111 | 0 | 0.124 | 0 |
| cds: Gen1_CTT C > T at MC1 cds % | 0 | 0.126 | 0 | 0.115 | 0 | 0.106 | 0 |
| cds: Gen3_GTC G > A at MC1 cds % | 0 | 0.261 | 0 | 0.296 | 0 | 0.256 | 0 |
| cds: Gen3_CTC G > A at MC1 cds % | 0 | 0.405 | 0 | 0.34 | 0 | 0.398 | 0 |
| cds: Gen3_ATC G > A at MC1 cds % | 0 | 0.222 | 0 | 0.248 | 0 | 0.186 | 0 |
| cds: Gen3_CCC G > C at MC1 cds % | 0 | 0.104 | 0 | 0.093 | 0 | 0.093 | 0 |
| cds: Gen3_CCC G > A at MC1 cds % | 0 | 0.283 | 0 | 0.265 | 0 | 0.323 | 0 |
| cds: Gen3_GAC G > T at MC1 cds % | 0 | 0.044 | 0 | 0.035 | 0 | 0.027 | 0 |
| cds: Gen3_CAC G > T at MC1 cds % | 0 | 0.104 | 0 | 0.128 | 0 | 0.093 | 0 |
| cds: Gen3_CAC G > A at MC1 cds % | 0 | 0.749 | 0 | 0.698 | 0 | 0.725 | 0 |
| cds: Gen3_AAC G > A at MC1 cds % | 0 | 0.431 | 0 | 0.389 | 0 | 0.354 | 0 |
| cds: ADAR_Gen3_GCA T > C at MC1 cds % | 0 | 0.27 | 0 | 0.296 | 0 | 0.301 | 0 |
| cds: ADAR_Gen3_AAA T > A at MC1 cds % | 0 | 0.048 | 0 | 0.044 | 0 | 0.044 | 0 |
| cds: ADAR_Gen2_AAA A > T at MC2 cds % | 0 | 0.03 | 0 | 0.031 | 0 | 0.013 | 0 |
| cds: ADAR_Gen2_AAC A > T at MC2 cds % | 0 | 0.039 | 0 | 0.031 | 0 | 0.031 | 0 |
| cds: Gen2_ACA C > T at MC2 cds % | 0 | 0.235 | 0 | 0.212 | 0 | 0.265 | 1 |
| cds: Gen2_ACG C > G at MC2 cds % | 0 | 0.039 | 0 | 0.053 | 0 | 0.04 | 0 |
| cds: Gen2_TCT G > C at MC2 cds % | 0 | 0.07 | 0 | 0.049 | 0 | 0.053 | 0 |
| cds: Gen2_TCT G > T at MC2 cds % | 0 | 0.026 | 0 | 0.035 | 0 | 0.018 | 0 |
| cds: Gen2_ACT G > A at MC2 cds % | 0 | 0.309 | 0 | 0.323 | 0 | 0.327 | 0 |
| cds: ADAR_Gen2_CAT A > G at MC2 cds % | 0 | 0.37 | 0 | 0.332 | 0 | 0.332 | 0 |
| cds: Gen2_TCG G > A at MC2 cds % | 0 | 0.414 | 0 | 0.358 | 0 | 0.389 | 0 |
| cds: Gen2_GCG G > T at MC2 cds % | 0 | 0.078 | 0 | 0.066 | 0 | 0.044 | 0 |
| cds: Gen2_CCG G > A at MC2 cds % | 0 | 0.771 | 0 | 0.765 | 0 | 0.8 | 0 |
| cds: Gen2_ACG G > C at MC2 cds % | 0 | 0.052 | 0 | 0.049 | 0 | 0.053 | 0 |
| cds: ADAR_Gen2_CAG T > C at MC2 cds % | 0 | 0.466 | 0 | 0.522 | 1 | 0.469 | 0 |
| cds: ADAR_Gen2_AAG T > C at MC2 cds % | 0 | 0.144 | 0 | 0.172 | 0 | 0.133 | 0 |
| cds: ADAR_Gen2_GAC A > C at MC2 cds % | 0 | 0.061 | 0 | 0.075 | 0 | 0.071 | 0 |
| cds: ADAR_Gen2_GAC A > T at MC2 cds % | 0 | 0.039 | 0 | 0.027 | 0 | 0.027 | 0 |
| cds: ADAR_Gen2_GAC A > G at MC2 cds % | 0 | 0.192 | 0 | 0.159 | 0 | 0.181 | 0 |
| cds: ADAR_Gen2_GAG A > C at MC2 cds % | 0 | 0.074 | 0 | 0.066 | 0 | 0.066 | 0 |
| cds: Gen2_GCA C > A at MC2 cds % | 0 | 0.087 | 0 | 0.115 | 1 | 0.071 | 0 |
| cds: Gen2_GCC C > A at MC2 cds % | 0 | 0.1 | 0 | 0.088 | 0 | 0.097 | 0 |
| cds: Gen2_GCG C > A at MC2 cds % | 0 | 0.039 | 0 | 0.057 | 0 | 0.071 | 0 |
| cds: Gen2_GCT C > T at MC2 cds % | 0 | 0.148 | 0 | 0.15 | 0 | 0.172 | 0 |
| cds: Gen2_GCC G > T at MC2 cds % | 0 | 0.07 | 0 | 0.057 | 0 | 0.075 | 0 |
| cds: Gen2_CCC G > A at MC2 cds % | 0 | 0.205 | 0 | 0.234 | 0 | 0.195 | 0 |
| cds: ADAR_Gen2_CAC T > A at MC2 cds % | 0 | 0.039 | 0 | 0.044 | 0 | 0.031 | 0 |
| cds: ADAR_Gen2_CAC T > C at MC2 cds % | 0 | 0.466 | 0 | 0.491 | 0 | 0.522 | 0 |
| cds: ADAR_Gen2_TAT A > G at MC2 cds % | 0 | 0.165 | 0 | 0.186 | 0 | 0.195 | 0 |
| cds: Gen2_TCT C > T at MC2 cds % | 0 | 0.091 | 0 | 0.071 | 0 | 0.053 | 0 |
| cds: Gen2_CCA G > A at MC2 cds % | 0 | 0.057 | 0 | 0.049 | 0 | 0.049 | 0 |
| cds: ADAR_Gen2_GAA T > A at MC2 cds % | 0 | 0.039 | 0 | 0.035 | 0 | 0.066 | 1 |
| cds: ADAR_Gen3_AAA A > T at MC3 cds % | 0 | 0.035 | 0 | 0.044 | 0 | 0.04 | 0 |
| cds: Gen3_ATC C > G at MC3 cds % | 0 | 0.074 | 0 | 0.084 | 0 | 0.075 | 0 |
| cds: Gen1_CAT G > A at MC3 cds % | 0 | 0.239 | 0 | 0.195 | 0 | 0.181 | 0 |
| cds: Gen3_CAC C > A at MC3 cds % | 0 | 0.03 | 0 | 0.062 | 0 | 0.075 | 0 |
| cds: Gen1_CTG G > C at MC3 cds % | 0 | 0.118 | 0 | 0.097 | 0 | 0.15 | 0 |
| cds: ADAR_Gen1_ATG T > G at MC3 cds % | 0 | 0.083 | 0 | 0.093 | 1 | 0.084 | 0 |
| cds: Gen3_GAC C > G at MC3 cds % | 0 | 0.135 | 0 | 0.133 | 0 | 0.137 | 0 |
| cds: Gen1_CTG G > C at MC3 cds % | 0 | 0.118 | 0 | 0.097 | 0 | 0.15 | 0 |
| cds: ADAR_Gen1_ATA T > G at MC3 cds % | 0 | 0.022 | 0 | 0.009 | 0 | 0.022 | 0 |
| cds: Gen3_TTC C > A at MC3 cds % | 0 | 0.039 | 0 | 0.04 | 0 | 0.035 | 0 |
| Total Scores: | 10 | | 17 | | 34 | | 41 |

S = score

TABLE 3

| Metric | Motif | Cutoff |
|---|---|---|
| cds: Gen2_TCT G > T at MC1 % | T-C-T | <22.7613 |
| cds: Gen2_TCT G > T at MC1 motif % | T-C-T | <1.0003 |
| cds: Gen2_TCC G > T at MC2 % | T-C-C | <19.7828 |
| cds: ADAR_Gen2_TAA T > G at MC1 % | T-A-A | <23.7942 |
| cds: ADAR_Gen2_CAA MC3 non-syn % | C-A-A | <1.5352 |
| cds: ADAR_Gen2_GAC A > G at MC2 motif % | G-A-C | <8.4447 |
| cds: AIDe G > T at MC2 motif % | WR-C-GW | <0.1539 |
| cds: ADARe A > C at MC1 % | CW-A-A | <16.6577 |
| cds: ADARj T > G at MC2 motif % | S-A-RA | <0.5120 |
| cds: A3Ge C > A at MC2 % | SC-C-GS | <9.0956 |
| cds: A3Ge C > A at MC2 motif % | SC-C-GS | <0.4283 |
| cds: A3Gf C > A at MC2 % | SC-C-G | <5.8307 |
| cds: A3Gf C > A at MC2 motif % | SC-C-G | <0.4548 |
| cds: A3Gg C > A at MC2 % | C-C-GS | <6.0242 |
| cds: A3Gg C > A at MC2 motif % | C-C-GS | <0.2493 |
| cds: A3Bc G > T at MC1 motif % | T-C-WA | <0.1310 |
| cds: ADAR_Gen1_AAC A > C at MC1 % | -A-AC | <17.0791 |
| cds: ADAR_Gen1_AAG A > T at MC1 % | -A-AG | <2.7397 |
| cds: ADAR_Gen1_ACG A > T at MC3 % | -A-CG | <32.6655 |
| cds: ADAR_Gen1_AGT T > G at MC1 % | -A-GT | <24.3459 |
| cds: ADAR_Gen1_AGT T > G at MC1 motif % | -A-GT | <1.5410 |
| cds: ADAR_Gen3_TAA A > C at MC3 % | TA-A- | <1.5008 |
| cds: ADAR_Gen3_TAA A > C at MC3 motif % | TA-A- | <0.2570 |
| cds: ADAR_Gen3_TGA A > C at MC3 % | TG-A- | <0.5264 |
| cds: ADAR_Gen3_CTA T > G at MC1 % | CT-A- | <0.3161 |
| cds: ADAR_Gen3_CTA T > G at MC1 motif % | CT-A- | <0.0112 |
| cds: Gen1_CTA G > C at MC1 % | -C-TA | <19.7933 |
| cds: Gen1_CAT C > A at MC1 motif % | -C-AT | <1.8052 |
| cds: Gen1_CGC C > A at MC2 % | -C-GC | <14.3891 |
| cds: Gen3_TAC C > G at MC3 motif % | TA-C- | <0.1909 |
| cds: Gen3_TTC G > T at MC1 motif % | TT-C- | <0.1494 |
| cds: Gen3_CGC C > G at MC2 % | CG-C- | <20.4021 |
| cds: Gen3_CGC C > G at MC2 motif % | CG-C- | <0.8523 |
| cds: AID G > T at MC1 % | WR-C- | >31.3533 |
| cds: Gen2_ACA C > A at MC2 % | A-C-A | >30.6654 |
| cds: Gen2_TCA C > A at MC2 % | T-C-A | >4.9596 |
| cds: Gen2_CCA C > A at MC2 % | C-C-A | >20.6352 |
| cds: ADAR_Gen2_AAA Ti A:T % | A-A-A | >65.0616 |
| cds: ADAR_Gen2_TAA T > G at MC3 motif % | T-A-A | >4.9319 |
| cds: ADAR_Gen2_GAC A > C at MC3 motif % | G-A-C | >3.0988 |
| cds: ADAR_Gen2_AAG A > T at MC2 % | A-A-G | >19.7189 |
| cds: A3Gi G > C at MC2 % | SG-C-G | >17.7726 |
| cds: A3Bb C > A at MC2 % | T-C-A | >4.9596 |
| cds: A3Bc G > C cds % | T-C-WA | >0.0758 |
| cds: A3Be C > A at MC2 % | YT-C-A | >5.9126 |
| cds: A3Be C > A at MC2 motif % | YT-C-A | >0.8986 |
| cds: A3Be C > A at MC2 cds % | YT-C-A | >0.0088 |
| cds: A3Bg G > T at MC3 motif % | T-C-GA | >0.8702 |
| cds: A3Bg G > T at MC3 cds % | T-C-GA | >0.0069 |
| cds: ADAR_Gen1_AAG Ti A:T % | -A-AG | >53.5190 |
| cds: ADAR_Gen3_ATA Ti A:T % | AT-A- | >42.0388 |
| cds: ADAR_Gen3_CAA A > G at MC1 % | CA-A- | >20.5971 |
| cds: ADAR_Gen3_GTA T > A at MC1 motif % | GT-A- | >1.3397 |
| cds: Gen1_CAT C > T at MC1 cds % | -C-AT | >0.1278 |
| cds: Gen1_CGC C > G at MC1 % | -C-GC | >31.7987 |
| cds: Gen1_CCG G > T at MC1 % | -C-CG | >25.4285 |
| cds: Gen1_CCG G > T at MC1 motif % | -C-CG | >2.3077 |
| cds: Gen3_TAC G > T at MC3 cds % | TA-C- | >0.0386 |
| cds: Gen3_CGC G > C % | CG-C- | >17.2163 |
| cds: Gen3_CGC C > G at MC1 % | CG-C- | >31.0006 |
| cds: Gen3_CGC C > A at MC2 motif % | CG-C- | >2.1080 |
| cds: Gen3_CGC C > G at MC1 motif % | CG-C- | >1.8182 |
| cds: Gen3_CGC C > A at MC2 cds % | CG-C- | >0.0388 |
| cds: Gen3_CGC C > G at MC1 cds % | CG-C- | >0.0326 |

TABLE 4

| Metric Name | Motif | Cutoff |
|---|---|---|
| cds: ADAR_Gen1_ATG T > C at MC2 % | -A-TG | >35.4426 |
| cds: ADAR_Gen3_ACA T > A motif % | AC-A- | >3.1037 |
| cds: Other MC2 G % | NA | >21.4661 |
| cds: Gen2_CCC G > T motif % | C-C-C | >8.2325 |
| cds: Gen3_GGC C > T motif % | GG-C- | >31.9160 |
| cds: ADAR_Gen2_GAG T > G cds % | G-A-G | >0.3916 |
| cds: Gen1_CTT C > A at MC2 cds % | -C-TT | >0.0312 |
| g: ADARf A > T + T > A g % | SW-A- | >1.3035 |
| cds: AIDc C > G at MC3 motif % | WR-C-GS | >1.1628 |
| cds: Gen3_AAC C > T at MC2 cds % | AA-C- | >0.2526 |
| g: Gen1_CCC C > T + G > A % | -C-CC | >59.8598 |
| cds: ADAR_Gen2_GAA MC2 % | G-A-A | >29.6598 |
| g: ADARe A > C + T > G g % | CW-A-A | >0.3277 |
| cds: ADAR_Gen1_ACG T > G at MC3 % | -A-CG | >74.1651 |
| cds: ADAR_Gen2_AAG T > A at MC3 cds % | A-A-G | >0.0520 |
| cds: Gen3_GAC G > A at MC1 motif % | GA-C- | >16.8174 |
| cds: ADAR_Gen1_AGC % | -A-GC | >3.8452 |
| g: ADAR_Gen2_GAA A > C + T > G g % | G-A-A | >0.5496 |
| cds: ADAR_Gen3_CAA A > C motif % | CA-A- | >6.2269 |
| cds: Gen1_CTC G > T at MC2 % | -C-TC | >27.9285 |
| cds: Gen3_CAC G > A at MC2 motif % | CA-C- | >11.3125 |
| cds: ADAR_Gen3_AAA A > T at MC3 % | AA-A- | >47.4258 |
| cds: ADAR_Gen2_GAG T > G motif % | G-A-G | >15.4476 |
| cds: Gen2_TCG G > A at MC2 cds % | T-C-G | >0.4283 |
| cds: ADAR_Gen1_ATG A > G at MC3 % | -A-TG | >25.7379 |
| cds: ADAR_Gen1_AAC A > C at MC2 motif % | -A-AC | >3.4295 |
| cds: Gen2_CCT G > C at MC3 motif % | C-C-T | >3.8401 |
| cds: Gen2_TCA C > A at MC1 % | T-C-A | >37.2674 |
| cds: A3Bb C > A at MC1 % | T-C-A | >37.2674 |
| cds: Gen3_TCC G > A at MC3 % | TC-C- | >70.1939 |
| cds: Gen1_CTT G > T at MC1 motif % | -C-TT | >2.0248 |
| cds: A3Bc C > T at MC2 motif % | T-C-WA | >7.6828 |
| cds: A3Bh G > A at MC3 cds % | WT-C-G | >0.2801 |
| cds: Gen2_GCC C > A at MC1 % | G-C-C | >40.6097 |
| cds: ADAR_Gen3_GAA A > C at MC2 cds % | GA-A- | >0.0977 |
| cds: ADARf A > C at MC2 % | SW-A- | >32.8535 |
| cds: A3Gg G > A cds % | C-C-GS | >2.0966 |
| cds: ADAR_Gen3_AGA T > G at MC2 % | AG-A- | >20.2595 |
| cds: A3Gc G > C % | C-C-GW | >11.5138 |
| cds: ADAR_Gen3_CGA A > T at MC1 motif % | CG-A- | >2.0403 |
| cds: ADAR_Gen1_AGC T > G at MC1 motif % | -A-GC | >1.5557 |
| cds: AIDf C > T motif % | WR-C-R | >42.1956 |
| cds: ADAR_Gen2_GAG non-syn % | G-A-G | >53.9812 |
| cds: Gen3_TCC G > A at MC3 cds % | TC-C- | >1.3527 |
| cds: ADAR_Gen2_GAC T > C at MC1 cds % | G-A-C | >0.1793 |
| cds: ADAR_Gen1_AGA A > G at MC1 motif % | -A-GA | >5.4330 |
| cds: ADAR_Gen3_GGA A > C at MC2 % | GG-A- | >35.6858 |
| g: ADARf A > C + T > G g % | SW-A- | >1.7375 |
| cds: ADARf A > C motif % | SW-A- | >7.4619 |
| cds: Gen2_ACC G > C at MC3 motif % | A-C-C | >2.6192 |
| cds: ADAR_Gen1_AGAT > C motif % | -A-GA | >30.6332 |
| cds: Gen3_CAC G > A at MC2 cds % | CA-C- | >0.5183 |
| g: Gen2_ACT % | A-C-T | >3.9808 |
| cds: Gen1_CTT G > A at MC1 % | -C-TT | >26.4353 |
| cds: ADAR_Gen1_ACT A > C at MC2 % | -A-CT | >26.7664 |
| cds: A3Bf C > A % | ST-C-G | >7.2355 |
| cds: ADAR_Gen3_GCA T > C % | GC-A- | >87.9071 |
| cds: ADAR_Gen3_GCA T Ti/Tv % | GC-A- | >87.9071 |
| cds: Gen1_CTT G > T at MC1 cds % | -C-TT | >0.0502 |
| cds: ADAR_Gen3_AGA T > G at MC2 motif % | AG-A- | >2.4877 |
| cds: Gen2_TCT C > A at MC2 % | T-C-T | >22.3425 |
| g: ADAR_Gen3_TGA % | TG-A- | >2.4975 |
| cds: ADARc A > C at MC2 % | SW-A-Y | >36.2229 |
| cds: Gen2_CCT G > C cds % | C-C-T | >0.2204 |
| cds: Gen1_CGT G > A at MC1 % | -C-GT | >35.6224 |
| cds: A3Bd C > A at MC1 % | RT-C-A | >36.7525 |
| cds: A3Bf C > A motif % | ST-C-G | >3.2529 |
| cds: A3Be MC3 % | YT-C-A | >65.9038 |
| g: ADARh A > T + T > A g % | W-A-S | >1.4383 |
| cds: Gen3_TAC C > T at MC1 % | TA-C- | >13.1726 |
| cds: Gen3_TGC C > G at MC2 % | TG-C- | >40.1384 |
| cds: ADAR_Gen1_AAT T > C at MC1 cds % | -A-AT | >0.0916 |
| cds: AIDb C > T at MC3 motif % | WR-C-G | >34.7430 |
| cds: ADAR_Gen2_TAT A > C at MC2 motif % | T-A-T | >1.3784 |
| cds: A3B G > C at MC3 motif % | T-C-W | >7.7288 |
| cds: A3Be C > T at MC3 % | YT-C-A | >69.9902 |
| cds: ADAR_Gen1_ATA T > A at MC2 cds % | -A-TA | >0.0216 |
| cds: Gen3_TCC C > A at MC1 % | TC-C- | >39.6359 |
| cds: ADAR_Gen2_GAG MC1 non-syn % | G-A-G | >90.1286 |
| cds: ADAR_Gen2_CAA MC1 % | C-A-A | >24.8308 |
| cds: ADAR_Gen2_CAA T > C at MC1 motif % | C-A-A | >4.7633 |
| cds: ADAR_Gen2_AAG T > A at MC3 motif % | A-A-G | >1.9140 |
| cds: ADAR_Gen2_CAA non-syn % | C-A-A | >44.7318 |
| cds: Gen3_AAC non-syn % | AA-C- | >50.1679 |
| cds: Gen2_CCT G > C motif % | C-C-T | >7.0084 |
| cds: Gen1_CGC G > A at MC2 cds % | -C-GC | >0.6493 |
| cds: Other G MC2 % | NA | >24.7577 |
| cds: Gen2_CCC C > A at MC1 motif % | C-C-C | >2.8885 |
| cds: Gen2_TCA C > A at MC1 motif % | T-C-A | >3.2036 |
| cds: A3Bb C > A at MC1 motif % | T-C-A | >3.2036 |
| cds: A3Gd G > C at MC3 cds % | SC-C-GW | >0.0547 |
| cds: ADAR_Gen1_AGA T > G at MC1 motif % | -A-GA | >2.4487 |
| g: ADAR_Gen2_TAG A > G + T > C g % | T-A-G | >1.4597 |
| cds: ADAR_Gen3_CTA T > A at MC3 motif % | CT-A- | >1.0434 |
| cds: Gen1_CAC G > C cds % | -C-AC | >0.2616 |
| cds: ADARd MC3 non-syn % | CW-A-Y | >7.6553 |
| cds: Gen2_ACC G > C at MC3 cds % | A-C-C | >0.0604 |
| cds: ADAR_Gen3_TGA A > T at MC1 motif % | TG-A- | >2.1090 |
| cds: ADAR_Gen3_AGA T > G at MC2 cds % | AG-A- | >0.0696 |
| g: Gen3_GTC C > G + G > C % | GT-C- | >23.8086 |
| cds: ADAR_Gen3_CAA A > C at MC2 cds % | CA-A- | >0.0977 |
| cds: ADAR_Gen3_GAA A > C motif % | GA-A- | >9.6823 |
| cds: Gen1_CGA C > G at MC3 % | -C-GA | >81.0147 |
| cds: AII G > C cds % | NA | >4.7572 |
| cds: Gen1_CAC G > C at MC3 cds % | -C-AC | >0.1861 |
| g: ADARc A > C + T > G g % | SW-A-Y | >0.8875 |
| cds: A3Bh G > A motif % | WT-C-G | >34.6865 |
| cds: A3Ge G > A at MC2 motif % | SC-C-GS | >14.0034 |
| g: ADAR_Gen1_ACG A > C + T > G % | -A-CG | >14.5412 |
| cds: Gen3_ACC G > C at MC2 motif % | AC-C- | >1.4825 |
| cds: Gen1_CTT G > C cds % | -C-TT | >0.2751 |
| g: ADAR_Gen1_AGT % | -A-GT | >2.6233 |
| cds: ADAR_Gen2_TAG T > G at MC1 cds % | T-A-G | >0.0173 |
| g: ADAR_Gen2_GAC % | G-A-C | >1.7872 |
| cds: ADAR_Gen2_AAG A > T at MC3 % | A-A-G | >69.8556 |
| g: ADAR_Gen1_ATG A > C + T > G % | -A-TG | >11.3915 |
| cds: Gen3_CTC Ti C:G % | CT-C- | >50.2596 |
| cds: Gen2_TCG G > A motif % | T-C-G | >39.1800 |
| cds: Gen1_CGT G > A at MC1 motif % | -C-GT | >14.2276 |
| cds: Gen3_CGC G > T at MC1 motif % | CG-C- | >3.7926 |
| cds: A3Bd C non-syn % | RT-C-A | >33.5758 |
| cds: ADAR_Gen1_ACA A > C at MC3 cds % | -A-CA | >0.1025 |
| cds: ADAR_Gen2_TAG T > G cds % | T-A-G | >0.0811 |
| g: Gen3_GAC C > G + G > C % | GA-C- | >18.4434 |
| cds: A3Be G > C at MC3 % | YT-C-A | >58.9178 |
| cds: Other AT Ti/Tv % | NA | >79.8280 |
| cds: Gen2_CCC C > G at MC1 % | C-C-C | >32.0521 |
| cds: Gen1_CTA G > C at MC2 motif % | -C-TA | >5.4731 |
| cds: ADAR_Gen3_CCA A > C at MC3 % | CC-A- | >52.4883 |
| g: ADAR_Gen2_TAG A > T + T > A g % | T-A-G | >0.2512 |
| g: Gen2_TCC % | T-C-C | >2.4225 |
| cds: ADAR_Gen1_ATG non-syn % | -A-TG | >67.5960 |
| cds: ADAR_Gen1_ATG T > C at MC2 motif % | -A-TG | >13.6550 |
| cds: Gen3_TAC G > A at MC1 % | TA-C- | >35.1759 |
| cds: ADAR_Gen3_TCA Ti/Tv % | TC-A- | >87.3124 |
| cds: Gen1_CAC G > C at MC3 motif % | -C-AC | >8.4899 |
| cds: A3Gg G > A motif % | C-C-GS | >43.3271 |
| cds: A3Bf G > A at MC2 % | ST-C-G | >33.2677 |
| cds: ADAR_Gen3_TTA A > G motif % | TT-A- | >36.2785 |
| cds: ADAR_Gen3_TTA A > G at MC3 cds % | TT-A- | >0.1905 |
| cds: Gen1_CTC G > C at MC2 cds % | -C-TC | >0.0945 |
| cds: ADAR_Gen1_ATT Ti/Tv % | -A-TT | >85.0354 |
| cds: ADAR_Gen3_GGA T > G at MC1 motif % | GG-A- | >6.5650 |
| cds: ADARj MC1 non-syn % | S-A-RA | >93.9883 |
| cds: ADAR_Gen2_AAG T > A motif % | A-A-G | >5.7246 |
| cds: ADAR_Gen3_CAA A > C at MC2 motif % | CA-A- | >2.6914 |
| cds: ADAR_Gen1_AAT A > T at MC2 % | -A-AT | >24.2682 |
| cds: Gen2_CCC G > T % | C-C-C | >20.0535 |
| cds: ADAR_Gen2_GAC A > T at MC1 cds % | G-A-C | >0.0483 |
| cds: ADARf A > C % | SW-A- | >12.6095 |
| cds: AIDe MC3 % | WR-C-GW | >67.8301 |
| g: ADAR_Gen2_CAG A > G + T > C g % | C-A-G | >3.0334 |
| cds: A3Bd MC1 % | RT-C-A | >27.4119 |
| cds: ADAR_Gen2_CAA T > C at MC1 cds % | C-A-A | >0.1521 |
| cds: ADAR_Gen3_GAA A > C cds % | GA-A- | >0.2434 |
| cds: ADAR_Gen2_GAC T > A motif % | G-A-C | >6.4968 |
| g: Gen1_CCA C > T + G > A % | -C-CA | >56.5987 |
| cds: A3Gg Ti/Tv % | C-C-GS | >79.2518 |
| cds: Gen3_CGC C > G at MC1 motif % | CG-C- | >1.9013 |
| cds: ADAR_Gen2_CAA T > C at MC1 % | C-A-A | >11.1764 |
| g: ADAR A > C + T > G g % | W-A- | >4.5932 |
| cds: ADAR_Gen3_CAA A > C % | CA-A- | >11.0981 |
| cds: ADAR_Gen1_ATT T > C % | -A-TT | >86.6456 |
| cds: ADAR_Gen1_ATT T Ti/Tv % | -A-TT | >86.6456 |
| g: ADARc A > T + T > A g % | SW-A-Y | >0.6766 |
| cds: A3G G > C motif % | C-C- | >7.0378 |
| cds: Gen1_CTG MC1 % | -C-TG | >37.8609 |
| cds: Gen1_CGC G > A at MC2 motif % | -C-GC | >10.5666 |
| cds: Gen3_CAC G > A cds % | CA-C- | >1.7065 |
| g: A3G C > A + G > T % | C-C- | >17.9468 |
| g: ADARc A > T + T > A % | SW-A-Y | >10.7902 |
| g: ADAR_Gen1_ACG A > C + T > G g % | -A-CG | >0.0756 |
| cds: ADAR_Gen1_AAT T > C at MC1 % | -A-AT | >12.3816 |
| cds: A3G G > C cds % | C-C- | >1.1945 |
| cds: ADAR A > C at MC2 % | W-A- | >28.8280 |
| g: ADAR_Gen3_CCA A > G + T > C g % | CC-A- | >3.3705 |
| cds: ADAR_Gen2_AAT A > C at MC2 % | A-A-T | >33.0447 |
| cds: Gen3_CGC C > G at MC1 cds % | CG-C- | >0.0344 |
| cds: ADARj T > A at MC2 % | S-A-RA | >58.4847 |
| cds: ADAR_Gen3_AGA MC2 % | AG-A- | >24.8523 |
| cds: A3Bh G > T at MC1 motif % | WT-C-G | >0.5023 |
| cds: ADAR_Gen3_ACA T > A % | AC-A- | >6.4309 |
| cds: ADAR_Gen3_CCA A > G at MC1 % | CC-A- | >32.6924 |
| cds: ADAR_Gen3_CCA A > G at MC1 motif % | CC-A- | >13.6789 |
| cds: A3Gh C > A at MC1 % | S-C-GS | >44.4006 |
| g: ADAR_Gen3_CAA A > C + T > G g % | CA-A- | >0.6021 |
| cds: ADAR_Gen2_AAG T > A cds % | A-A-G | >0.1554 |
| cds: Gen2_CCA MC3 non-syn % | C-C-A | >9.7508 |
| cds: ADAR_Gen1_AGA T > A at MC2 % | -A-GA | >43.2056 |
| g: ADAR_Gen2_GAC A > G + T > C g % | G-A-C | >1.1361 |
| cds: ADAR_Gen3_CCA A > G at MC1 cds % | CC-A- | >0.9178 |
| cds: ADAR_Gen1_AGG Ti % | -A-GG | >2.8254 |
| cds: ADAR_Gen2_CAG A > G % | C-A-G | >82.0040 |
| cds: ADAR_Gen2_CAG A Ti/Tv % | C-A-G | >82.0040 |
| cds: A3Bc G > C at MC2 motif % | T-C-WA | >3.3367 |
| cds: Gen1_CTT G > T at MC1 % | -C-TT | >38.0644 |
| cds: Other C MC2 Ti/Tv % | NA | >70.2047 |
| g: ADAR_Gen1_AGA A > T + T > A g % | -A-GA | >0.4026 |
| cds: AIDg G > A at MC3 cds % | AG-C-TNT | >0.0218 |
| cds: ADAR_Gen1_ATG T > C at MC2 cds % | -A-TG | >0.5442 |
| cds: ADAR_Gen3_CAA A > C at MC3 cds % | CA-A- | >0.0725 |
| cds: ADAR_Gen3_CAA A > C at MC3 motif % | CA-A- | >1.9908 |
| cds: Gen1_CTA Ti C:G % | -C-TA | >70.1024 |
| cds: Gen1_CTA C:G % | -C-TA | >71.2708 |
| cds: Gen3_ACC G > C at MC3 motif % | AC-C- | >5.4877 |
| cds: Gen3_CGC C > G at MC1 % | CG-C- | >29.7120 |
| cds: Gen1_CTA G > C % | -C-TA | >32.9430 |
| g: ADAR_Gen1_AAG A > G + T > C g % | -A-AG | >1.9094 |
| cds: AIDb C:G % | WR-C-G | >54.6407 |
| cds: ADAR_Gen3_TTA MC1 non-syn % | TT-A- | >98.6826 |
| cds: Gen1_CCG G > C % | -C-CG | >30.9165 |
| cds: ADAR_Gen3_CGA MC1 % | CG-A- | >26.4968 |
| cds: Gen1_CTA G > C at MC2 % | -C-TA | >56.2616 |
| cds: ADARi T > C at MC3 motif % | RAW-A- | >24.3585 |
| cds: A3Bd non-syn % | RT-C-A | >40.8951 |
| g: ADARd A > T + T > A % | CW-A-Y | >9.5401 |
| g: ADAR_Gen3_CAA A > C + T > G % | CA-A- | >16.4789 |
| g: ADAR_Gen2_GAG A > T + T > A g % | G-A-G | >0.3441 |
| cds: A3Bh G > T at MC1 cds % | WT-C-G | >0.0087 |
| cds: Gen3_TAC MC1 % | TA-C- | >24.1172 |
| cds: Gen1_CCG C > A at MC1 % | -C-CG | >19.8324 |
| cds: ADAR_Gen1_AAT A > T at MC2 motif % | -A-AT | >1.1662 |
| cds: ADAR_Gen3_GGA T > G at MC1 % | GG-A- | >56.0574 |
| cds: Gen3_GAC C > G % | GA-C- | >14.0340 |
| cds: A3Gd G > C at MC3 % | SC-C-GW | >65.3998 |
| cds: ADAR_Gen3_CTA T > G at MC3 cds % | CT-A- | >0.0557 |
| cds: Gen1_CCG G > C motif % | -C-CG | >15.2481 |
| cds: ADAR_Gen3_TCA T > G at MC1 motif % | TC-A- | >0.2008 |
| cds: ADAR_Gen3_GCA A > T at MC2 % | GC-A- | >29.3242 |
| cds: ADAR_Gen3_TGA A > T at MC1 % | TG-A- | >46.5742 |
| cds: ADAR_Gen3_TCA T > G at MC1 % | TC-A- | >5.1723 |
| g: Gen2_ACC C > T + G > A % | A-C-C | >55.4933 |
| cds: Gen2_CCT G > C % | C-C-T | >14.1381 |
| cds: A3Gc G > C at MC3 motif % | C-C-GW | >3.8867 |
| cds: Gen1_CAC G > C motif % | -C-AC | >11.9398 |
| cds: ADAR_Gen3_TGA MC1 non-syn % | TG-A- | >96.3630 |
| g: ADAR_Gen3_TGA A > G + T > C g % | TG-A- | >1.5505 |
| cds: Gen3_GAC G > A motif % | GA-C- | >35.1782 |
| cds: ADAR_Gen1_AGA MC1 % | -A-GA | >19.9105 |
| cds: Gen3_CGC G > T at MC1 % | CG-C- | >46.1628 |
| cds: AIDb Ti C:G % | WR-C-G | >57.2423 |
| cds: ADAR_Gen3_TCA T > G at MC1 cds % | TC-A- | >0.0086 |
| cds: ADAR_Gen2_CAG T > C % | C-A-G | >83.7074 |
| cds: ADAR_Gen2_CAG T Ti/Tv % | C-A-G | >83.7074 |
| cds: Gen2_GCT C:G % | G-C-T | >44.1791 |
| cds: Gen1_CGT G > A at MC1 cds % | -C-GT | >0.7622 |
| cds: AIDb C > T motif % | WR-C-G | >48.6086 |
| cds: Gen3_GAC non-syn % | GA-C- | >51.7361 |
| cds: ADAR_Gen3_AGA A non-syn % | AG-A- | >75.9139 |
| cds: Gen1_CCT G > A at MC2 % | -C-CT | >23.1853 |
| cds: A3Bf non-syn % | ST-C-G | >52.9219 |
| g: Gen1_CCA C > T + G > A g % | -C-CA | >1.6983 |
| g: Gen2_GCA % | G-C-A | >2.5114 |
| cds: Gen1_CGC G > A at MC2 % | -C-GC | >26.1125 |
| g: ADARh % | W-A-S | >9.1846 |
| g: ADARd A > T + T > A g % | CW-A-Y | >0.3733 |
| g: AIDb C > T + G > A % | WR-C-G | <80.8417 |
| cds: A3F Ti % | T-C- | <6.5646 |
| cds: ADAR_Gen1_AGG T > G at MC3 motif % | -A-GG | <2.9409 |
| cds: Gen2_CCC G > A motif % | C-C-C | <23.7708 |
| cds: ADAR_Gen2_AAA T > A at MC2 cds % | A-A-A | <0.0278 |
| cds: ADAR_Gen1_ATT T > G cds % | -A-TT | <0.1082 |
| g: ADAR_Gen3_TGA A > T + T > A % | TG-A- | <17.4515 |
| g: A3Ge C > T + G > A g % | SC-C-GS | <1.0379 |
| cds: ADARd T > G at MC3 cds % | CW-A-Y | <0.0365 |
| cds: Gen2_ACC C:G % | A-C-C | <58.9436 |
| cds: A3Gg C > A cds % | C-C-GS | <0.2138 |
| cds: Other A MC3 % | NA | <43.3163 |
| cds: Gen1_CAA G > T at MC2 % | -C-AA | <9.4295 |
| cds: Gen3_CTC G > A at MC3 motif % | CT-C- | <11.6939 |
| cds: ADARe Hits | CW-A-A | <227.1468 |
| cds: A3Ge C > A at MC2 motif % | SC-C-GS | <0.6639 |
| cds: Gen1_CTA G > A at MC2 cds % | -C-TA | <0.0920 |
| g: ADAR_Gen3_CCA A > C + T > G % | CC-A- | <12.8723 |
| cds: ADAR_Gen1_ACG T > G at MC1 motif % | -A-CG | <1.0451 |
| cds: Gen1_CTT G > A % | -C-TT | <71.7698 |
| cds: Gen1_CTT G Ti/Tv % | -C-TT | <71.7698 |
| g: Gen1_CTC C > T + G > A g % | -C-TC | <1.8086 |
| cds: ADAR_Gen2_GAT A > T at MC3 cds % | G-A-T | <0.0185 |
| cds: A3Bc MC1 % | T-C-WA | <28.0281 |
| cds: ADAR_Gen1_AGG A > T cds % | -A-GG | <0.1425 |
| cds: Gen2_GCC C > A at MC2 % | G-C-C | <31.3454 |
| cds: Gen1_CAC G > A % | -C-AC | <61.6429 |
| cds: Gen1_CAC G Ti/Tv % | -C-AC | <61.6429 |
| cds: Gen3_TGC non-syn % | TG-C- | <58.6169 |
| cds: ADAR_Gen1_AAA T > A at MC3 cds % | -A-AA | <0.0108 |
| cds: ADAR_Gen3_GAA T > A at MC2 motif % | GA-A- | <0.5484 |
| cds: ADAR_Gen3_GAA T > A at MC2 cds % | GA-A- | <0.0138 |
| cds: A3Gg C > A at MC3 cds % | C-C-GS | <0.1020 |
| cds: ADAR_Gen2_AAA A > C at MC1 motif % | A-A-A | <1.7457 |
| cds: Gen3_GAC C > T at MC3 cds % | GA-C- | <1.2607 |
| cds: AII MC2 C:G % | NA | <49.2861 |
| g: Gen1_CTA % | -C-TA | <2.3948 |
| g: AIDe C > T + G > A % | WR-C-GW | <80.3324 |
| cds: Gen3_TTC C > T motif % | TT-C- | <34.5218 |
| cds: A3Be Hits | YT-C-A | <224.5469 |
| cds: A3Be non-syn % | YT-C-A | <43.3291 |
| g: Gen1_CTA C > G + G > C g % | -C-TA | <0.6118 |
| cds: Gen3_TTC % | TT-C- | <2.5492 |
| cds: Gen2_CCC C > G at MC2 motif % | C-C-C | <2.0048 |
| cds: ADAR_Gen3_AGA T > A at MC1 motif % | AG-A- | <1.2586 |
| cds: ADAR_Gen1_AGG T > G at MC3 cds % | -A-GG | <0.1099 |
| cds: ADAR_Gen2_TAA T > A at MC3 cds % | T-A-A | <0.0200 |
| cds: ADARh A > T at MC1 motif % | W-A-S | <0.9328 |
| cds: Gen3_CTC G > A at MC3 % | CT-C- | <36.2740 |
| cds: Gen3_GAC C > A at MC3 cds % | GA-C- | <0.0894 |
| cds: AIDg G > T cds % | AG-C-TNT | <0.0073 |
| cds: ADARj A > T cds % | S-A-RA | <0.0775 |
| cds: A3B C > T at MC2 cds % | T-C-W | <0.1497 |
| g: Gen1_CGT % | -C-GT | <3.8937 |
| cds: ADAR_Gen1_ACG T > G at MC1 cds % | -A-CG | <0.0138 |
| cds: ADAR_Gen1_ATT T > G motif % | -A-TT | <3.7386 |
| cds: Other C:G % | NA | <50.3658 |
| cds: Gen3_CAC C non-syn % | CA-C- | <56.3417 |
| cds: ADAR_Gen1_ATT non-syn % | -A-TT | <44.7330 |
| cds: ADAR_Gen3_GGA T > G at MC3 motif % | GG-A- | <3.0160 |
| cds: A3Bh C:G % | WT-C-G | <57.2837 |
| cds: ADAR_Gen1_ATT T > G at MC2 cds % | -A-TT | <0.0230 |
| cds: Gen3_ATC G > T at MC3 cds % | AT-C- | <0.0446 |
| cds: Gen1_CCA C > A at MC3 cds % | -C-CA | <0.0965 |
| cds: A3Bc % | T-C-WA | <0.5525 |
| cds: Gen3_TTC C:G % | TT-C- | <46.7058 |
| cds: A3Gh C > A at MC2 motif % | S-C-GS | <0.9684 |
| cds: ADARg T > A at MC1 motif % | W-A-A | <1.3303 |
| cds: Gen3_CAC C > G % | CA-C- | <15.7001 |
| cds: Gen2_GCG C non-syn % | G-C-G | <42.2847 |
| cds: ADAR_Gen3_GCA T > G % | GC-A- | <6.9424 |
| g: A3F % | T-C- | <11.7184 |
| cds: Gen3_GTC Hits | GT-C- | <429.2249 |
| cds: A3Bh Ti C:G % | WT-C-G | <59.1895 |
| cds: ADAR_Gen3_GGA A > C at MC3 % | GG-A- | <45.3433 |
| cds: Gen3_GCC C > G at MC2 motif % | GC-C- | <0.8421 |
| cds: Gen3_GAC MC3 % | GA-C- | <55.7514 |
| cds: Gen2_ACT C > G at MC2 % | A-C-T | <41.8522 |
| cds: Gen3_TCC G > A at MC2 % | TC-C- | <18.1634 |
| cds: ADAR_Gen3_AGA T > A at MC1 cds % | AG-A- | <0.0351 |
| cds: Gen1_CTA Hits | -C-TA | <218.5357 |
| cds: ADAR_Gen2_CAG T > G % | C-A-G | <10.0239 |
| cds: Gen3_ATC G > T at MC3 motif % | AT-C- | <2.0905 |
| g: AIDe % | WR-C-GW | <2.4061 |
| cds: Gen2_CCC C > G at MC2 cds % | C-C-C | <0.0588 |
| cds: ADAR_Gen2_TAA T > A at MC3 motif % | T-A-A | <1.6612 |
| cds: AIDg G > T % | AG-C-TNT | <8.8961 |
| cds: Gen3_GCC C > G at MC2 cds % | GC-C- | <0.0378 |
| cds: Gen2_GCT G > T at MC3 cds % | G-C-T | <0.0520 |
| g: ADARb A > G + T > C g % | W-A-Y | <9.0549 |
| g: Gen2_TCC C > A + G > T % | T-C-C | <17.6193 |
| cds: A3Ge C > A motif % | SC-C-GS | <4.5418 |
| cds: ADAR_Gen2_CAG T > G at MC3 cds % | C-A-G | <0.2068 |
| cds: Gen3_GAC C > A at MC3 % | GA-C- | <44.7160 |
| g: Gen3_CTC C > T + G > A g % | CT-C- | <2.0722 |
| cds: Gen2_TCC Hits | T-C-C | <516.8439 |
| cds: Gen2_GCT G > T at MC3 motif % | G-C-T | <1.8797 |
| cds: Gen3_CAC C > G cds % | CA-C- | <0.3510 |
| cds: Gen1_CGC G > A at MC1 % | -C-GC | <26.1467 |
| cds: ADAR_Gen1_ATT T non-syn % | -A-TT | <36.8912 |
| cds: Gen3_CAC C > G at MC2 % | CA-C- | <36.6174 |
| cds: ADAR_Gen1_AAC Hits | -A-AC | <363.6953 |
| cds: Gen3_TTC C > T at MC3 cds % | TT-C- | <0.6066 |
| cds: ADAR_Gen3_CAA A > T at MC2 % | CA-A- | <38.3576 |
| cds: Gen1_CTA G > A cds % | -C-TA | <0.1711 |
| cds: ADAR_Gen3_TCA T > G % | TC-A- | <6.1351 |
| cds: A3Be C non-syn % | YT-C-A | <38.6414 |
| cds: Gen3_TGC C non-syn % | TG-C- | <58.7478 |
| g: Gen3_GTC C > T + G > A % | GT-C- | <60.1254 |
| cds: Gen1_CTA G > C at MC1 % | -C-TA | <28.8759 |
| cds: Gen3_CTC G > A at MC3 cds % | CT-C- | <0.3835 |
| cds: Other C % | NA | <20.7626 |
| cds: Gen2_TCG C > T cds % | T-C-G | <1.5229 |
| cds: ADAR_Gen2_CAG T > G cds % | C-A-G | <0.3349 |
| cds: ADAR_Gen2_GAT A > C at MC3 cds % | G-A-T | <0.0399 |
| cds: AIDb G > T at MC2 % | WR-C-G | <14.2198 |
| cds: ADAR_Gen1_ATG T > C at MC3 % | -A-TG | <47.9641 |
| g: Gen3_AGC C > T + G > A % | AG-C- | <60.2503 |
| cds: ADAR_Gen1_AGC Ti/Tv % | -A-GC | <77.5244 |
| cds: Gen1_CTC C > G at MC3 cds % | -C-TC | <0.1000 |
| cds: ADAR_Gen1_ATT MC2 % | -A-TT | <15.3628 |
| cds: A3Gc G > A % | C-C-GW | <81.0704 |
| cds: A3Gc G Ti/Tv % | C-C-GW | <81.0704 |
| cds: ADAR_Gen1_ATG MC3 % | -A-TG | <33.9466 |
| cds: AIDb G > A at MC3 motif % | WR-C-G | <17.1367 |
| cds: Gen2_TCA C > T at MC2 cds % | T-C-A | <0.0698 |
| cds: A3Bb C > T at MC2 cds % | T-C-A | <0.0698 |
| cds: ADAR_Gen2_CAG T > G at MC3 motif % | C-A-G | <2.9287 |
| g: ADAR A > G + T > C % | W-A- | <63.7014 |
| cds: ADAR_Gen3_GCA T > A at MC3 cds % | GC-A- | <0.0545 |
| cds: ADARg T > A at MC1 cds % | W-A-A | <0.0400 |
| cds: ADAR_Gen1_AAC A > C at MC1 motif % | -A-AC | <1.3221 |
| g: Gen2_CCT C > G + G > C g % | C-C-T | <0.4999 |
| cds: ADAR_Gen1_AGG A > T % | -A-GG | <7.8811 |
| cds: Gen2_ACG C > G at MC1 cds % | A-C-G | <0.0250 |
| cds: Gen2_CCC C > G at MC2 % | C-C-C | <13.9069 |
| cds: ADAR_Gen2_GAT T > G at MC1 cds % | G-A-T | <0.0556 |
| cds: ADAR_Gen1_AAC A > C at MC1 cds % | -A-AC | <0.0209 |
| cds: A3Be MC1 % | YT-C-A | <25.0504 |
| cds: ADAR_Gen1_ATC A > T at MC3 % | -A-TC | <48.5158 |
| cds: Gen3_GAC C > A at MC3 motif % | GA-C- | <2.1691 |
| cds: Gen3_TGC C > A at MC2 motif % | TG-C- | <2.2153 |
| cds: Gen1_CGG C > A at MC2 % | -C-GG | <20.4042 |
| cds: ADAR_Gen3_TCA T > G at MC3 cds % | TC-A- | <0.1047 |
| cds: ADAR_Gen3_TCA T > G at MC3 motif % | TC-A- | <2.3814 |
| cds: ADARd A > G at MC3 cds % | CW-A-Y | <0.6551 |
| cds: A3Gg C > A at MC3 motif % | C-C-GS | <2.1266 |
| cds: A3Bc C > T at MC1 cds % | T-C-WA | <0.0273 |
| cds: ADAR_Gen3_TCA T > G cds % | TC-A- | <0.1555 |
| g: Gen2_ACC C > A + G > T % | A-C-C | <26.9932 |
| cds: Other T > G % | NA | <11.5506 |
| cds: Gen3_TAC G > T at MC3 cds % | TA-C- | <0.0154 |
| cds: ADAR_Gen1_ATG A > G at MC3 motif % | -A-TG | <10.3290 |
| cds: Gen1_CTA G > C at MC1 cds % | -C-TA | <0.0234 |
| cds: ADAR_Gen2_CAG T > G motif % | C-A-G | <4.7500 |
| cds: ADAR_Gen3_TAA A > G at MC2 % | TA-A- | <42.5026 |
| cds: ADAR_Gen1_AGG A > T motif % | -A-GG | <3.8151 |
| cds: ADAR_Gen3_CTA Hits | CT-A- | <506.7259 |
| cds: A3Gh C > A at MC2 % | S-C-GS | <19.3238 |
| cds: Gen1_CCC C > G motif % | -C-CC | <10.7948 |
| g: ADARf A > G + T > C % | SW-A- | <72.7058 |
| cds: Gen1_CCA C > A at MC3 motif % | -C-CA | <4.0503 |
| cds: ADARh A > T at MC1 cds % | W-A-S | <0.0750 |
| cds: Gen3_GAC C > T at MC3 motif % | GA-C- | <30.8464 |
| cds: ADAR_Gen3_GCA T > A at MC3 motif % | GC-A- | <1.0861 |
| cds: ADAR_Gen3_TCA T > G motif % | TC-A- | <3.5352 |
| g: ADAR_Gen3_CAA A > G + T > C % | CA-A- | <70.5850 |
| cds: Gen3_TCC C > A motif % | TC-C- | <5.2016 |
| g: Gen1_CGT C > T + G > A % | -C-GT | <80.3023 |
| g: ADARc A > G + T > C % | SW-A-Y | <75.0672 |
| cds: AIDb G > A at MC3 cds % | WR-C-G | <0.9652 |
| cds: Gen3_CAC C > G at MC2 motif % | CA-C- | <2.9407 |
| cds: Gen2_TCG C:G % | T-C-G | <50.5576 |
| g: Gen2_CCT C > G + G > C % | C-C-T | <16.8662 |
| cds: Gen3_GAC Ti C:G % | GA-C- | <54.3999 |
| cds: Gen3_CAC C > G at MC2 cds % | CA-C- | <0.1340 |
| cds: A3Bh C > T cds % | WT-C-G | <0.8832 |
| cds: ADAR_Gen3_TAA Hits | TA-A- | <199.9503 |
| cds: Gen2_TCA C > A at MC2 cds % | T-C-A | <0.0032 |
| cds: A3Bb C > A at MC2 cds % | T-C-A | <0.0032 |
| cds: A3Bf MC3 % | ST-C-G | <46.3578 |
| cds: A3Bc Hits | T-C-WA | <130.1122 |
| cds: Gen1_CCA C > A at MC3 % | -C-CA | <37.1091 |
| cds: Gen2_TCA C > A at MC2 motif % | T-C-A | <0.1938 |
| cds: A3Bb C > A at MC2 motif % | T-C-A | <0.1938 |
| cds: Gen3_TTC C > T cds % | TT-C- | <0.8959 |
| g: ADARd A > G + T > C % | CW-A-Y | <76.8510 |

TABLE 5

| Metric Name | Motif | Cutoff |
|---|---|---|
| cds: ADAR_Gen1_ATG T > C at MC2 % | -A-TG | >35.4426 |
| cds: ADAR_Gen3_ACA T > A motif % | AC-A- | >3.1037 |
| g: ADARf A > T + T > A g % | SW-A- | >1.3035 |
| g: ADARe A > C + T > G g % | CW-A-A | >0.3277 |
| cds: ADAR_Gen1_ACG T > G at MC3 % | -A-CG | >74.1651 |
| cds: ADAR_Gen2_AAG T > A at MC3 cds % | A-A-G | >0.0520 |
| cds: ADAR_Gen1_AGC % | -A-GC | >3.8452 |
| g: ADAR_Gen2_GAA A > C + T > G g % | G-A-A | >0.5496 |
| cds: ADAR_Gen3_CAA A > C motif % | CA-A- | >6.2269 |
| cds: ADAR_Gen3_AAA A > T at MC3 % | AA-A- | >47.4258 |
| cds: ADAR_Gen2_GAG T > G motif % | G-A-G | >15.4476 |
| cds: ADAR_Gen1_ATG A > G at MC3 % | -A-TG | >25.7379 |
| cds: ADAR_Gen1_AAC A > C at MC2 motif % | -A-AC | >3.4295 |
| cds: ADAR_Gen3_GAA A > C at MC2 cds % | GA-A- | >0.0977 |
| cds: ADARf A > C at MC2 % | SW-A- | >32.8535 |
| cds: ADAR_Gen3_AGA T > G at MC2 % | AG-A- | >20.2595 |
| cds: ADAR_Gen3_CGA A > T at MC1 motif % | CG-A- | >2.0403 |
| cds: ADAR_Gen1_AGC T > G at MC1 motif % | -A-GC | >1.5557 |
| cds: ADAR_Gen2_GAG non-syn % | G-A-G | >53.9812 |
| cds: ADAR_Gen2_GAC T > C at MC1 cds % | G-A-C | >0.1793 |
| cds: ADAR_Gen1_AGA A > G at MC1 motif % | -A-GA | >5.4330 |
| cds: ADAR_Gen3_GGA A > C at MC2 % | GG-A- | >35.6858 |
| g: ADARf A > C + T > G g % | SW-A- | >1.7375 |
| cds: ADARf A > C motif % | SW-A- | >7.4619 |
| cds: ADAR_Gen1_AGA T > C motif % | -A-GA | >30.6332 |
| cds: ADAR_Gen1_ACT A > C at MC2 % | -A-CT | >26.7664 |
| cds: ADAR_Gen3_GCA T > C % | GC-A- | >87.9071 |
| cds: ADAR_Gen3_GCA T Ti/Tv % | GC-A- | >87.9071 |
| cds: ADAR_Gen3_AGA T > G at MC2 motif % | AG-A- | >2.4877 |
| g: ADAR_Gen3_TGA % | TG-A- | >2.4975 |
| cds: ADARc A > C at MC2 % | SW-A-Y | >36.2229 |
| g: ADARh A > T + T > A g % | W-A-S | >1.4383 |
| cds: ADAR_Gen1_AAT T > C at MC1 cds % | -A-AT | >0.0916 |
| cds: AIDb C > T at MC3 motif % | WR-C-G | >34.7430 |
| cds: ADAR_Gen2_TAT A > C at MC2 motif % | T-A-T | >1.3784 |
| cds: ADAR_Gen1_ATA T > A at MC2 cds % | -A-TA | >0.0216 |
| cds: ADAR_Gen2_GAG MC1 non-syn % | G-A-G | >90.1286 |
| cds: ADAR_Gen2_CAA MC1 % | C-A-A | >24.8308 |
| cds: ADAR_Gen2_CAA T > C at MC1 motif % | C-A-A | >4.7633 |
| cds: ADAR_Gen2_AAG T > A at MC3 motif % | A-A-G | >1.9140 |
| cds: ADAR_Gen2_CAA non-syn % | C-A-A | >44.7318 |
| cds: ADAR_Gen1_AGA T > G at MC1 motif % | -A-GA | >2.4487 |
| g: ADAR_Gen2_TAG A > G + T > C g % | T-A-G | >1.4597 |
| cds: ADAR_Gen3_CTA T > A at MC3 motif % | CT-A- | >1.0434 |
| cds: ADARd MC3 non-syn % | CW-A-Y | >7.6553 |
| cds: ADAR_Gen3_TGA A > T at MC1 motif % | TG-A- | >2.1090 |
| cds: ADAR_Gen3_AGA T > G at MC2 cds % | AG-A- | >0.0696 |
| cds: ADAR_Gen3_CAA A > C at MC2 cds % | CA-A- | >0.0977 |
| cds: ADAR_Gen3_GAA A > C motif % | GA-A- | >9.6823 |
| g: ADARc A > C + T > G g % | SW-A-Y | >0.8875 |
| g: ADAR_Gen1_ACG A > C + T > G % | -A-CG | >14.5412 |
| g: ADAR_Gen1_AGT % | -A-GT | >2.6233 |
| cds: ADAR_Gen2_TAG T > G at MC1 cds % | T-A-G | >0.0173 |
| g: ADAR_Gen2_GAC % | G-A-C | >1.7872 |
| cds: ADAR_Gen2_AAG A > T at MC3 % | A-A-G | >69.8556 |
| g: ADAR_Gen1_ATG A > C + T > G % | -A-TG | >11.3915 |
| cds: ADAR_Gen1_ACA A > C at MC3 cds % | -A-CA | >0.1025 |
| cds: ADAR_Gen2_TAG T > G cds % | T-A-G | >0.0811 |
| cds: ADAR_Gen3_CCA A > C at MC3 % | CC-A- | >52.4883 |
| g: ADAR_Gen2_TAG A > T + T > A g % | T-A-G | >0.2512 |
| cds: ADAR_Gen1_ATG non-syn % | -A-TG | >67.5960 |
| cds: ADAR_Gen1_ATG T > C at MC2 motif % | -A-TG | >13.6550 |
| cds: ADAR_Gen3_TCA Ti/Tv % | TC-A- | >87.3124 |
| cds: ADAR_Gen3_TTA A > G motif % | TT-A- | >36.2785 |
| cds: ADAR_Gen3_TTA A > G at MC3 cds % | TT-A- | >0.1905 |
| cds: ADAR_Gen1_ATT Ti/Tv % | -A-TT | >85.0354 |
| cds: ADAR_Gen3_GGA T > G at MC1 motif % | GG-A- | >6.5650 |
| cds: ADARj MC1 non-syn % | S-A-RA | >93.9883 |
| cds: ADAR_Gen2_AAG T > A motif % | A-A-G | >5.7246 |
| cds: ADAR_Gen3_CAA A > C at MC2 motif % | CA-A- | >2.6914 |
| cds: ADAR_Gen1_AAT A > T at MC2 % | -A-AT | >24.2682 |
| cds: ADAR_Gen2_GAC A > T at MC1 cds % | G-A-C | >0.0483 |
| cds: ADARf A > C % | SW-A- | >12.6095 |
| cds: AIDe MC3 % | WR-C-GW | >67.8301 |
| g: ADAR_Gen2_CAG A > G + T > C g % | C-A-G | >3.0334 |
| cds: ADAR_Gen2_CAA T > C at MC1 cds % | C-A-A | >0.1521 |
| cds: ADAR_Gen3_GAA A > C cds % | GA-A- | >0.2434 |
| cds: ADAR_Gen2_GAC T > A motif % | G-A-C | >6.4968 |
| cds: ADAR_Gen2_CAA T > C at MC1 % | C-A-A | >11.1764 |
| g: ADAR A > C + T > G g % | W-A- | >4.5932 |
| cds: ADAR_Gen3_CAA A > C % | CA-A- | >11.0981 |
| cds: ADAR_Gen1_ATT T > C % | -A-TT | >86.6456 |
| cds: ADAR_Gen1_ATT T Ti/Tv % | -A-TT | >86.6456 |
| g: ADARc A > T + T > A g % | SW-A-Y | >0.6766 |
| g: ADARc A > T + T > A % | SW-A-Y | >10.7902 |
| g: ADAR_Gen1_ACG A > C + T > G g % | -A-CG | >0.0756 |
| cds: ADAR_Gen1_AAT T > C at MC1 % | -A-AT | >12.3816 |
| cds: ADAR A > C at MC2 % | W-A- | >28.8280 |
| g: ADAR_Gen3_CCA A > G + T > C g % | CC-A- | >3.3705 |
| cds: ADAR_Gen2_AAT A > C at MC2 % | A-A-T | >33.0447 |
| cds: ADARj T > A at MC2 % | S-A-RA | >58.4847 |
| cds: ADAR_Gen3_AGA MC2 % | AG-A- | >24.8523 |
| cds: ADAR_Gen3_ACA T > A % | AC-A- | >6.4309 |
| cds: ADAR_Gen3_CCA A > G at MC1 % | CC-A- | >32.6924 |
| cds: ADAR_Gen3_CCA A > G at MC1 motif % | CC-A- | >13.6789 |
| g: ADAR_Gen3_CAA A > C + T > G g % | CA-A- | >0.6021 |
| cds: ADAR_Gen2_AAG T > A cds % | A-A-G | >0.1554 |
| cds: ADAR_Gen1_AGA T > A at MC2 % | -A-GA | >43.2056 |
| g: ADAR_Gen2_GAC A > G + T > C g % | G-A-C | >1.1361 |
| cds: ADAR_Gen3_CCA A > G at MC1 cds % | CC-A- | >0.9178 |
| cds: ADAR_Gen1_AGG Ti % | -A-GG | >2.8254 |
| cds: ADAR_Gen2_CAG A > G % | C-A-G | >82.0040 |
| cds: ADAR_Gen2_CAG A Ti/Tv % | C-A-G | >82.0040 |
| g: ADAR_Gen1_AGA A > T + T > A g % | -A-GA | >0.4026 |
| cds: ADAR_Gen1_ATG T > C at MC2 cds % | -A-TG | >0.5442 |
| cds: ADAR_Gen3_CAA A > C at MC3 cds % | CA-A- | >0.0725 |
| cds: ADAR_Gen3_CAA A > C at MC3 motif % | CA-A- | >1.9908 |
| g: ADAR_Gen1_AAG A > G + T > C g % | -A-AG | >1.9094 |
| cds: ADAR_Gen3_TTA MC1 non-syn % | TT-A- | >98.6826 |
| cds: ADAR_Gen3_CGA MC1 % | CG-A- | >26.4968 |
| cds: ADARi T > C at MC3 motif % | RAW-A- | >24.3585 |
| g: ADARd A > T + T > A % | CW-A-Y | >9.5401 |
| g: ADAR_Gen3_CAA A > C + T > G % | CA-A- | >16.4789 |
| g: ADAR_Gen2_GAG A > T + T > A g % | G-A-G | >0.3441 |
| cds: ADAR_Gen1_AAT A > T at MC2 motif % | -A-AT | >1.1662 |
| cds: ADAR_Gen3_GGA T > G at MC1 % | GG-A- | >56.0574 |
| cds: ADAR_Gen3_CTA T > G at MC3 cds % | CT-A- | >0.0557 |
| cds: ADAR_Gen3_TCA T > G at MC1 motif % | TC-A- | >0.2008 |
| cds: ADAR_Gen3_GCA A > T at MC2 % | GC-A- | >29.3242 |
| cds: ADAR_Gen3_TGA A > T at MC1 % | TG-A- | >46.5742 |
| cds: ADAR_Gen3_TCA T > G at MC1 % | TC-A- | >5.1723 |
| cds: ADAR_Gen3_TGA MC1 non-syn % | TG-A- | >96.3630 |
| g: ADAR_Gen3_TGA A > G + T > C g % | TG-A- | >1.5505 |
| cds: ADAR_Gen1_AGA MC1 % | -A-GA | >19.9105 |
| cds: ADAR_Gen3_TCA T > G at MC1 cds % | TC-A- | >0.0086 |
| cds: ADAR_Gen2_CAG T > C % | C-A-G | >83.7074 |
| cds: ADAR_Gen2_CAG T Ti/Tv % | C-A-G | >83.7074 |
| cds: AIDb C > T motif % | WR-C-G | >48.6086 |
| cds: ADAR_Gen3_AGA A non-syn % | AG-A- | >75.9139 |
| g: ADARh % | W-A-S | >9.1846 |
| g: ADARd A > T + T > A g % | CW-A-Y | >0.3733 |
| cds: ADAR_Gen1_AGG T > G at MC3 motif % | -A-GG | <2.9409 |
| cds: ADAR_Gen2_AAA T > A at MC2 cds % | A-A-A | <0.0278 |
| cds: ADAR_Gen1_ATT T > G cds % | -A-TT | <0.1082 |
| g: ADAR_Gen3_TGA A > T + T > A % | TG-A- | <17.4515 |
| cds: ADARd T > G at MC3 cds % | CW-A-Y | <0.0365 |
| cds: ADARe Hits | CW-A-A | <227.1468 |
| g: ADAR_Gen3_CCA A > C + T > G % | CC-A- | <12.8723 |
| cds: ADAR_Gen1_ACG T > G at MC1 motif % | -A-CG | <1.0451 |
| cds: ADAR_Gen2_GAT A > T at MC3 cds % | G-A-T | <0.0185 |
| cds: ADAR_Gen1_AGG A > T cds % | -A-GG | <0.1425 |
| cds: ADAR_Gen1_AAA T > A at MC3 cds % | -A-AA | <0.0108 |
| cds: ADAR_Gen3_GAA T > A at MC2 motif % | GA-A- | <0.5484 |
| cds: ADAR_Gen3_GAA T > A at MC2 cds % | GA-A- | <0.0138 |
| cds: ADAR_Gen2_AAA A > C at MC1 motif % | A-A-A | <1.7457 |
| cds: ADAR_Gen3_AGA T > A at MC1 motif % | AG-A- | <1.2586 |
| cds: ADAR_Gen1_AGG T > G at MC3 cds % | -A-GG | <0.1099 |
| cds: ADAR_Gen2_TAA T > A at MC3 cds % | T-A-A | <0.0200 |
| cds: ADARh A > T at MC1 motif % | W-A-S | <0.9328 |
| cds: ADARj A > T cds % | S-A-RA | <0.0775 |
| cds: ADAR_Gen1_ACG T > G at MC1 cds % | -A-CG | <0.0138 |
| cds: ADAR_Gen1_ATT T > G motif % | -A-TT | <3.7386 |
| cds: ADAR_Gen1_ATT non-syn % | -A-TT | <44.7330 |
| cds: ADAR_Gen3_GGA T > G at MC3 motif % | GG-A- | <3.0160 |
| cds: ADAR_Gen1_ATT T > G at MC2 cds % | -A-TT | <0.0230 |
| cds: ADARg T > A at MC1 motif % | W-A-A | <1.3303 |
| cds: ADAR_Gen3_GCA T > G % | GC-A- | <6.9424 |
| cds: ADAR_Gen3_GGA A > C at MC3 % | GG-A- | <45.3433 |
| cds: ADAR_Gen3_AGA T > A at MC1 cds % | AG-A- | <0.0351 |
| cds: ADAR_Gen2_CAG T > G % | C-A-G | <10.0239 |
| cds: ADAR_Gen2_TAA T > A at MC3 motif % | T-A-A | <1.6612 |
| g: ADARb A > G + T > C g % | W-A-Y | <9.0549 |
| cds: ADAR_Gen2_CAG T > G at MC3 cds % | C-A-G | <0.2068 |
| cds: ADAR_Gen1_ATT T non-syn % | -A-TT | <36.8912 |
| cds: ADAR_Gen1_AAC Hits | -A-AC | <363.6953 |
| cds: ADAR_Gen3_CAA A > T at MC2 % | CA-A- | <38.3576 |
| cds: ADAR_Gen3_TCA T > G % | TC-A- | <6.1351 |
| cds: ADAR_Gen2_CAG T > G cds % | C-A-G | <0.3349 |
| cds: ADAR_Gen2_GAT A > C at MC3 cds % | G-A-T | <0.0399 |
| cds: ADAR_Gen1_ATG T > C at MC3 % | -A-TG | <47.9641 |
| cds: ADAR_Gen1_AGC Ti/Tv % | -A-GC | <77.5244 |
| cds: ADAR_Gen1_ATT MC2 % | -A-TT | <15.3628 |
| cds: ADAR_Gen1_ATG MC3 % | -A-TG | <33.9466 |
| cds: ADAR_Gen2_CAG T > G at MC3 motif % | C-A-G | <2.9287 |
| g: ADAR A > G + T > C % | W-A- | <63.7014 |
| cds: ADAR_Gen3_GCA T > A at MC3 cds % | GC-A- | <0.0545 |
| cds: ADARg T > A at MC1 cds % | W-A-A | <0.0400 |
| cds: ADAR_Gen1_AAC A > C at MC1 motif % | -A-AC | <1.3221 |
| cds: ADAR_Gen1_AGG A > T % | -A-GG | <7.8811 |
| cds: ADAR_Gen2_GAT T > G at MC1 cds % | G-A-T | <0.0556 |
| cds: ADAR_Gen1_AAC A > C at MC1 cds % | -A-AC | <0.0209 |
| cds: ADAR_Gen1_ATC A > T at MC3 % | -A-TC | <48.5158 |
| cds: ADAR_Gen3_TCA T > G at MC3 cds % | TC-A- | <0.1047 |
| cds: ADAR_Gen3_TCA T > G at MC3 motif % | TC-A- | <2.3814 |
| cds: ADARd A > G at MC3 cds % | CW-A-Y | <0.6551 |
| cds: ADAR_Gen3_TCA T > G cds % | TC-A- | <0.1555 |
| cds: ADAR_Gen1_ATG A > G at MC3 motif % | -A-TG | <10.3290 |
| cds: ADAR_Gen2_CAG T > G motif % | C-A-G | <4.7500 |
| cds: ADAR_Gen3_TAA A > G at MC2 % | TA-A- | <42.5026 |
| cds: ADAR_Gen1_AGG A > T motif % | -A-GG | <3.8151 |
| cds: ADAR_Gen3_CTA Hits | CT-A- | <506.7259 |
| g: ADARf A > G + T > C % | SW-A- | <72.7058 |
| cds: ADARh A > T at MC1 cds % | W-A-S | <0.0750 |
| cds: ADAR_Gen3_GCA T > A at MC3 motif % | GC-A- | <1.0861 |
| cds: ADAR_Gen3_TCA T > G motif % | TC-A- | <3.5352 |
| g: ADAR_Gen3_CAA A > G + T > C % | CA-A- | <70.5850 |
| g: ADARc A > G + T > C % | SW-A-Y | <75.0672 |
| cds: AIDb G > A at MC3 cds % | WR-C-G | <0.9652 |
| cds: ADAR_Gen3_TAA Hits | TA-A- | <199.9503 |
| g: ADARd A > G + T > C % | CW-A-Y | <76.8510 |

TABLE 6

| Metric Name | Motif | Cutoff |
|---|---|---|
| cds: Gen3_ACC G > C at MC3 motif % | AC-C- | >5.4877 |
| cds: Gen3_CGC C > G at MC1 % | CG-C- | >29.7120 |
| cds: Gen1_CTA G > C % | -C-TA | >32.9430 |
| cds: AIDb C:G % | WR-C-G | >54.6407 |
| cds: Gen1_CCG G > C % | -C-CG | >30.9165 |
| cds: ADAR_Gen3_CGA MC1 % | CG-A- | >26.4968 |
| cds: Gen1_CTA G > C at MC2 % | -C-TA | >56.2616 |
| cds: ADARi T > C at MC3 motif % | RAW-A- | >24.3585 |
| cds: A3Bd non-syn % | RT-C-A | >40.8951 |
| g: ADARd A > T + T > A % | CW-A-Y | >9.5401 |
| cds: Gen3_TAC MC1 % | TA-C- | >24.1172 |
| cds: Gen1_CCG C > A at MC1 % | -C-CG | >19.8324 |
| cds: ADAR_Gen1_AAT A > T at MC2 motif % | -A-AT | >1.1662 |
| cds: ADAR_Gen3_GGA T > G at MC1 % | GG-A- | >56.0574 |
| cds: Gen3_GAC C > G % | GA-C- | >14.0340 |
| cds: A3Gd G > C at MC3 % | SC-C-GW | >65.3998 |
| cds: Gen1_CCG G > C motif % | -C-CG | >15.2481 |
| cds: ADAR_Gen3_GCA A > T at MC2 % | GC-A- | >29.3242 |
| cds: ADAR_Gen3_TGA A > T at MC1 % | TG-A- | >46.5742 |
| cds: ADAR_Gen3_TCA T > G at MC1 % | TC-A- | >5.1723 |
| g: Gen2_ACC C > T + G > A % | A-C-C | >55.4933 |
| cds: Gen2_CCT G > C % | C-C-T | >14.1381 |
| cds: A3Gc G > C at MC3 motif % | C-C-GW | >3.8867 |
| cds: Gen1_CAC G > C motif % | -C-AC | >11.9398 |
| cds: ADAR_Gen3_TGA MC1 non-syn % | TG-A- | >96.3630 |
| cds: Gen3_GAC G > A motif % | GA-C- | >35.1782 |
| cds: ADAR_Gen1_AGA MC1 % | -A-GA | >19.9105 |
| cds: Gen3_CGC G > T at MC1 % | CG-C- | >46.1628 |
| cds: AIDb Ti C:G % | WR-C-G | >57.2423 |
| cds: ADAR_Gen2_CAG T > C % | C-A-G | >83.7074 |
| cds: ADAR_Gen2_CAG T Ti/Tv % | C-A-G | >83.7074 |
| cds: Gen2_GCT C:G % | G-C-T | >44.1791 |
| cds: Gen1_CGT G > A at MC1 cds % | -C-GT | >0.7622 |
| cds: AIDb C > T motif % | WR-C-G | >48.6086 |
| cds: Gen3_GAC non-syn % | GA-C- | >51.7361 |
| cds: ADAR_Gen3_AGA A non-syn % | AG-A- | >75.9139 |
| cds: Gen1_CCT G > A at MC2 % | -C-CT | >23.1853 |
| cds: A3Bf non-syn % | ST-C-G | >52.9219 |
| g: Gen1_CCA C > T + G > A g % | -C-CA | >1.6983 |
| cds: Gen1_CGC G > A at MC2 % | -C-GC | >26.1125 |
| g: ADARd A > T + T > A g % | CW-A-Y | >0.3733 |
| cds: ADAR_Gen3_TCA T > G at MC3 cds % | TC-A- | <0.104731 |
| cds: ADAR_Gen3_TCA T > G at MC3 motif % | TC-A- | <2.381411 |
| cds: ADARd A > G at MC3 cds % | CW-A-Y | <0.655084 |
| cds: A3Gg C > A at MC3 motif % | C-C-GS | <2.126605 |
| cds: ADAR_Gen3_TCA T > G cds % | TC-A- | <0.155519 |
| g: Gen2_ACC C > A + G > T % | A-C-C | <26.99317 |
| cds: Other T > G % | NA | <11.55061 |
| cds: Gen3_TAC G > T at MC3 cds % | TA-C- | <0.015401 |
| cds: ADAR_Gen1_ATG A > G at MC3 motif % | -A-TG | <10.32899 |
| cds: ADAR_Gen2_CAG T > G motif % | C-A-G | <4.74997 |
| cds: ADAR_Gen3_TAA A > G at MC2 % | TA-A- | <42.50258 |
| cds: ADAR_Gen1_AGG A > T motif % | -A-GG | <3.815128 |
| cds: A3Gh C > A at MC2 % | S-C-GS | <19.32381 |
| cds: Gen1_CCC C > G motif % | -C-CC | <10.79479 |
| g: ADARf A > G + T > C % | SW-A- | <72.70582 |
| cds: Gen1_CCA C > A at MC3 motif % | -C-CA | <4.050294 |
| cds: ADARh A > T at MC1 cds % | W-A-S | <0.075042 |
| cds: Gen3_GAC C > T at MC3 motif % | GA-C- | <30.84638 |
| cds: ADAR_Gen3_GCA T > A at MC3 motif % | GC-A- | <1.086123 |
| cds: ADAR_Gen3_TCA T > G motif % | TC-A- | <3.535247 |
| cds: Gen3_TCC C > A motif % | TC-C- | <5.201635 |
| cds: AIDb G > A at MC3 cds % | WR-C-G | <0.965247 |
| cds: Gen3_CAC C > G at MC2 motif % | CA-C- | <2.940721 |
| cds: Gen2_TCG C:G % | T-C-G | <50.55757 |
| cds: Gen3_GAC Ti C:G % | GA-C- | <54.39992 |
| cds: Gen3_CAC C > G at MC2 cds % | CA-C- | <0.133976 |
| cds: A3Bh C > T cds % | WT-C-G | <0.883189 |
| cds: A3Bf MC3 % | ST-C-G | <46.35784 |
| cds: Gen1_CCA C > A at MC3 % | -C-CA | <37.10905 |
| cds: Gen3_TTC C > T cds % | TT-C- | <0.895855 |

The disclosure of every patent, patent application, and publication cited herein is hereby incorporated herein by reference in its entirety.

The citation of any reference herein should not be construed as an admission that such reference is available as “Prior Art” to the instant application.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Throughout the specification the aim has been to describe the preferred embodiments of the invention without limiting the invention to any one embodiment or specific collection of features. Those of skill in the art will therefore appreciate that, in light of the instant invention, various modifications and changes can be made in the particular embodiments exemplified without departing from the scope of the present invention. All such modifications and changes are intended to be included within the scope of the appended claims.

1. A method for determining the likelihood that a subject has or will develop a neurodegenerative disease, comprising:
analyzing the sequence of a nucleic acid molecule from a subject to detect SNVs within the nucleic acid molecule;
determining a plurality of metrics based on the number and/or type of SNVs detected so as to obtain a subject profile of metrics; and,
determining the likelihood of a subject having or developing a neurodegenerative disease on a comparison between the subject profile and a reference profile of metrics;
wherein:
the neurodegenerative disease is mild cognitive impairment (MCI) or Alzheimer's disease (AD) and the plurality of metrics comprises those set forth in Table 1, or at least 90% of the metrics set forth in Table 1;
the neurodegenerative disease is early mild cognitive impairment (EMCI) and the plurality of metrics comprises those set forth in Table 2, or at least 90% of the metrics set forth in Table 2;
the neurodegenerative disease is AD and the plurality of metrics is comprises those set forth in Table 3, or at least 90% of the metrics set forth in Table 3; or
the neurodegenerative disease is Parkinson's disease (PD) and the plurality of metrics is comprises those set forth in any one of Tables 4-6, or at least 90% of the metrics set forth in any one of Tables 4-6.

2. The method of claim 1, wherein the reference profile is representative of a subject that has or will develop the neurodegenerative disease.

3. The method of claim 1, wherein the comparison includes:
(i) assigning a score to each metric that that is outside a predetermined range interval, or above or below a predetermined cut-off, for the metric;
(ii) combining each score to calculate a total score; and
(iii) comparing the total score to a predetermined threshold score;
wherein the subject is determined to be likely to have or to develop the neurodegenerative disease when the total score is equal to or more than, or is more than, the threshold score.

4. The method of claim 1, wherein the sequence is a whole genome or whole exome sequence.

5. The method of claim 1, wherein the nucleic acid molecule was obtained from blood, saliva or nasal swab.

6. A method for treating a neurodegerative disease in a subject, the method comprising:
(i) performing the method according to claim 1;
(ii) determining that the subject is likely to have a neurodegenerative disease selected from among MCI, EMCI, Alzheimer's disease and Parkinson's disease; and
(iii) exposing the subject to a therapy.

7. The method of claim 6, wherein the disease is MCI, EMCI or Alzheimer's disease and therapy comprises administration of a cognitive enhancer, an anti-inflammatory, an anti-neuropsychiatric, a cholinesterase inhibitor, an N-methyl-D-aspartate receptor antagonist, an anti-beta amyloid agent (A(3) agent, and/or an anti-tau agent.

8. The method of claim 7, wherein therapy comprises administration of one or more of donepezil, galantamine, rivastigmine, memantine, Aducanumab, levetiracetam, ALZT-OP1, cromolyn+ibuprofen, blarcamesine, AVP-786, AXS-05, Azeliragon, BAN2401, troriluzole, BPDO-1603, Brexpiprazole, CAD106b, COR388, Escitalopram, Gantenerumab, Gantenerumab and solanezumab, Ginkgo biloba, Guanfacine, Icosapent ethyl (IPE), Losartan+amlodipine+atorvastatin, Masitinib, Metformin, Methylphenidate, Mirtazapine, Octohydro-aminoacridine Succinate, Solanezumab, Tricaprilin, TRx0237, or Zolpidem+zoplicone.

9. The method of claim 6, wherein the disease is Parkinson's disease and therapy comprises administration of levodopa, a dopamine agonist (e.g. bromocriptine, cabergoline, apomorphine, pramipexole, ropinirole, or rotigotine), a monoamine oxidase-B (MAO B) inhibitor (e.g. selegiline, rasagiline or safinamide), a catechol O-methyltransferase (COMT) inhibitor (e.g. entacapone or tolcapone), an anticholinergic (e.g. enztropine or trihexyphenidyl), amantadine, an adenosine A2A antagonist (e.g. istradefylline), Cu-ATSM, a cell therapy (e.g. mesenchymal stem cells, or neural stem cells), a kinase inhibitor (e.g. DNL 151, FB-101, saracatinib), a neurotropic factor (e.g. GDNF or CDNF), or a GLP-1 agonist (e.g. exenatide).
